(19) United States
(12) Patent Application Publication
(10) Pub. No.: US 2022/0331313 A1
(43) Pub. Date: Oct. 20, 2022

(54) METHODS FOR TREATING SARCOIDOSIS-ASSOCIATED PULMONARY HYPERTENSION

(71) Applicant: ACTELION PHARMACEUTICALS LTD, Allschwil (CN)

(72) Inventors: Ralph T. PREISS, Allschwil (CH); Tatiana REMENOVA, Allschwil (CH); Irina KONOURINA, Allschwil (CH); Thomas PFISTER, Allschwil (CH); René ROSCHER, Allschwil (CH); Aline FREY, Allschwil (CH); Natalia Catalina YANNOULIS ARCEDA, Allschwil (CH); Rainer ZIMMERMANN, Allschwil (CH); Keith John MORRISON, Allschwil (CH); Shirin BRUDERER, Allschwil (CH)

(21) Appl. No.: 17/608,820

(22) PCT Filed: May 6, 2020
(86) PCT No.: PCT/EP2020/062568

§ 371 Date: Nov. 4, 2021

Related U.S. Application Data
(60) Provisional application No. 62/843,848, filed on May 6, 2019.

(51) Int. Cl. A61K 31/4965; A61P 9/12

(52) U.S. Cl. CPC A61K 31/4965; A61P 9/12; A61K 9/20

(57) ABSTRACT

The present disclosure provides methods for treating sarcoidosis-associated pulmonary hypertension, comprising administering to a patient in need thereof, a therapeutically effective amount of selexipag, wherein the patient is diagnosed with sarcoidosis-associated pulmonary hypertension.

### CROSS-REFERENCE TO RELATED APPLICATIONS

This application claims the benefit of the priority of U.S. Provisional Patent Application No. 62/843,848, filed 6 May 2019, the disclosure of which is incorporated herein in its entirety.

### FIELD OF THE INVENTION

This invention relates to methods for treating subjects with sarcoidosis-associated pulmonary hypertension.

### BACKGROUND

Sarcoidosis is a multisystemic disorder that is characterized by non-caseating granulomas which are present in multiple tissues, particularly in the lung and lymphatic system. Pulmonary hypertension (defined as a mean pulmonary artery pressure [PAP] of ≥25 mmHg at rest measured by right heart catheterization [RHC]) is increasingly recognized as a serious complication of pulmonary sarcoidosis and is associated with an increased morbidity and mortality, particularly precapillary sarcoidosis-associated pulmonary hypertension (SAPH). The World Health Organization (WHO) generally classifies SAPH as Group 5 (i.e., PH with an unknown and multifactorial mechanism). Severe untreated pulmonary hypertension (PH) carries a poor prognosis and is associated with higher mortality in patients with interstitial lung diseases and sarcoidosis. Early diagnosis and consideration of treatment options may be key for improving patient outcomes. There are no clear data regarding the true prevalence of SAPH. The proportion of sarcoidosis patients with PH varies from 5% to 20%.

While there is no approved treatment for SAPH, PH-specific treatments are frequently used, including endothelin receptor antagonists (ERA; e.g., bosentan, ambrisentan, macitentan), phosphodiesterase type-5 inhibitors (PDESi; e.g., sildenafil), guanylate cyclase agonist (e.g., riociguat), and prostacyclin analogues (e.g., i.v. epoprostenol or inhaled iloprost). There is evidence from small studies and case series that PH-specific therapies reduce pulmonary vascular resistance (PVR) and improve other pulmonary hemodynamic parameters in patients with SAPH. Some of these studies also showed an improvement in WHO functional class (FC), exercise capacity, or Quality of Life (QoL).

Despite the current standard of care, there are no specific treatments for patients with SAPH. What is needed are methods of treating patients having SAPH.

### SUMMARY

In some aspects, the disclosure is directed to treating patients with sarcoidosis-associated pulmonary hypertension. The methods comprise administering a therapeutically effective amount of selexipag.

In further aspects, the disclosure is directed to methods for treating sarcoidosis-associated pulmonary hypertension in a patient in need thereof, comprising (a) determining if the patient has sarcoidosis-associated pulmonary hypertension; and (b) if the patient has sarcoidosis-associated pulmonary hypertension, administering a therapeutically effective amount of selexipag.

### DESCRIPTION OF THE DRAWINGS

FIG. 1 is a schematic overview of the study described in Example 2.

FIG. 2 is schematic of the titration phase design described in Example 2. In this figure, D=days, V=visit; and W=week.

FIG. 3 is a schematic overview of the study described in Example 3.

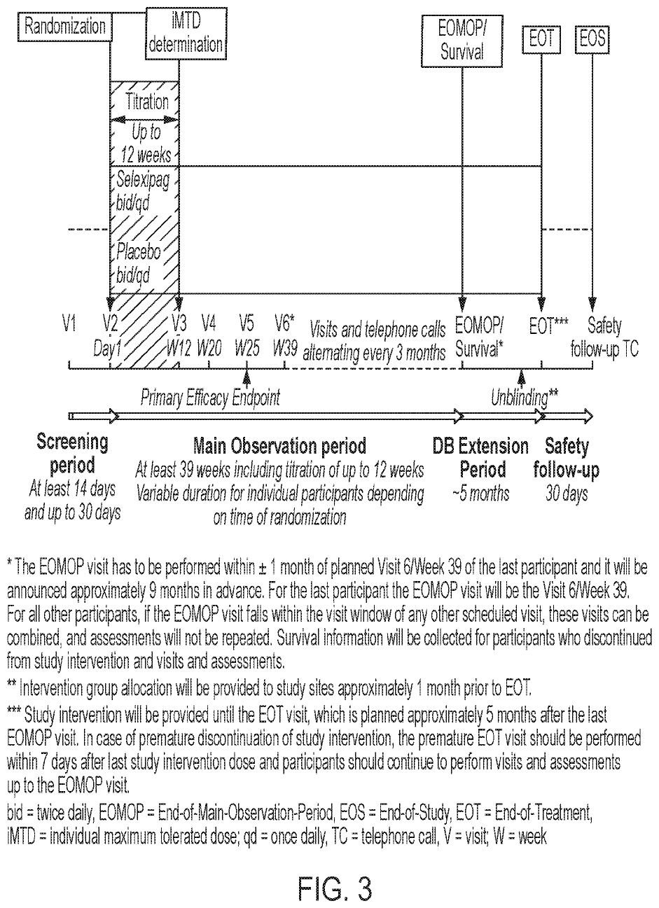

FIG. 4 is a schematic of the titration period described in Example 3.

### DETAILED DESCRIPTION OF ILLUSTRATIVE EMBODIMENTS

In the present disclosure the singular forms “a”, “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” or “substantially” it will be understood that the particular value forms another embodiment. In general, use of the term “about” or “substantially” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about” or “substantially”. In other cases, the gradations used in a series of values may be used to determine the intended range available to the term “about” or “substantially” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiments and such a combination is considered to be another embodiment. Conversely, various features of the invention that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

### Methods

The present disclosure provides methods for treating sarcoidosis-associated pulmonary hypertension (SAPH), comprising administering to a patient in need thereof, a therapeutically effective amount of selexipag. The methods described herein reflect the effectiveness and safety of selexipag in treating a specific subpopulation of patients having pulmonary hypertension, i.e., those having sarcoidosis-associated pulmonary hypertension.

The terms “sarcoidosis-associated pulmonary hypertension” and “SAPH” are interchangeable and define the condition in which a patent has pulmonary hypertension and sarcoidosis. Typically, SAPH is classified by the World Health Organization (WHO) using a functional classification of pulmonary hypertension as described below. The methods described herein may include a determination that the patient has SAPH. Typically, that determination is made by an attending physician. A diagnosis or determination of SAPH may be performed using techniques known by those of skill in the art. For example, a right-heart catheterization may be conducted to confirm pulmonary hypertension in a patient with sarcoidosis.

In some embodiments, prior to initiating treatment with selexipag, a patient having SAPH has a forced vital capacity (FVC) of greater than (>) about 50%. In some embodiments, the patient has a FVC of about 50, about 55, about 60, about 65, about 70, about 75, about 80, about 85, about 90, about 95, or about 99%. In further embodiments, the patient has a FVC of about 50 to about 99, about 50 to about 95, about 50 to about 90, about 50 to about 85, about 50 to about 80, about 50 to about 75, about 50 to about 70, about 50 to about 65, about 50 to about 60, about 50 to about 55, about 55 to about 99, about 55 to about 95, about 55 to about 90, about 55 to about 85, about 55 to about 80, about 55 to about 75, about 55 to about 70, about 55 to about 65, about 55 to about 60, about 60 to about 99, about 60 to about 95, about 60 to about 90, about 60 to about 85, about 60 to about 80, about 60 to about 75, about 60 to about 70, about 60 to about 65, about 65 to about 99, about 65 to about 95, about 65 to about 90, about 65 to about 85, about 65 to about 80, about 65 to about 75, about 65 to about 70, about 70 to about 99, about 70 to about 95, about 70 to about 90, about 70 to about 85, about 70 to about 80, about 70 to about 75, about 75 to about 99, about 75 to about 95, about 75 to about 90, about 75 to about 85, about 75 to about 80, about 80 to about 99, about 80 to about 95, about 80 to about 90, about 80 to about 85, about 95 to about 99, about 85 to about 95, about 85 to about 90, about 90 to about 99, or about 95 to about 99%.

In other embodiments, prior to initiating treatment with selexipag, a patient having SAPH has a forced expiratory volume (FEV₁) of greater than about 30% in about 1 second. In some embodiments, the patient has a FEV₁ of about 30, about 35, about 40, about 45, about 50, about 55, about 60, about 65, about 70, about 75, about 80, about 85, about 90, about 95, or about 99% in one second. In further embodiments, the patient has a FEV₁ of about 30 to about 99, about 30 to about 95, about 30 to about 90, about 30 to about 85, about 30 to about 80, about 30 to about 80, about 30 to about 75, about 30 to about 70, about 30 to about 65, about 30 to about 60, about 30 to about 55, about 30 to about 50, about 30 to about 45, about 30 to about 40, about 30 to about 35, about 35 to about 99, about 35 to about 95, about 35 to about 90, about 35 to about 85, about 35 to about 80, about 35 to about 75, about 35 to about 70, about 35 to about 65, about 35 to about 60, about 35 to about 55, about 35 to about 50, about 35 to about 45, about 35 to about 40, about 40 to about 99, about 40 to about 95, about 40 to about 90, about 40 to about 85, about 40 to about 80, about 40 to about 75, about 40 to about 70, about 40 to about 65, about 40 to about 60, about 40 to about 55, about 40 to about 50, about 45 to about 50, about 45 to about 99, about 45 to about 95, about 45 to about 95, about 45 to about 90, about 45 to about 85, about 45 to about 80, about 45 to about 75, about 45 to about 70, about 45 to about 65, about 45 to about 60, about 45 to about 55, about 45 to about 50, about 50 to about 99, about 50 to about 95, about 50 to about 90, about 50 to about 85, about 50 to about 80, about 50 to about 75, about 50 to about 70, about 50 to about 65, about 50 to about 60, about 50 to about 55, about 55 to about 99, about 55 to about 95, about 55 to about 90, about 55 to about 85, about 55 to about 80, about 55 to about 75, about 55 to about 70, about 55 to about 65, about 55 to about 60, about 60 to about 99, about 60 to about 95, about 60 to about 90, about 60 to about 85, about 60 to about 80, about 60 to about 75, about 60 to about 70, about 60 to about 65, about 65 to about 99, about 65 to about 95, about 65 to about 90, about 65 to about 85, about 65 to about 80, about 65 to about 75, about 65 to about 70, about 70 to about 99, about 70 to about 95, about 70 to about 90, about 70 to about 85, about 70 to about 80, about 70 to about 75, about 75 to about 99, about 75 to about 95, about 75 to about 90, about 75 to about 85, about 75 to about 80, about 80 to about 99, about 80 to about 95, about 80 to about 90, about 80 to about 85, about 95 to about 99, about 85 to about 95, about 85 to about 90, about 90 to about 99, or about 95 to about 99% in about 1 second. In some embodiments, a patient with SAPH has a FVC≥about 50% and a FEV₁ (in about 1 second)≥30%. In other embodiments, a patient with SAPH has FEV₁/FVC of about 60% or greater, or if FEV₁/FVC is about 60% or less, then FEV₁ is about 60% or greater.

The methods described herein advantageously reduce the patient's PVR relative to a patient population at the same or similar level of disease diagnosis that is not receiving treatment with selexipag (placebo). Such a relative analysis is disclosed in the Examples herein. In some embodiments, prior to initiating treatment with selexipag, SAPH patients have a pulmonary vascular resistance (PVR)≥about 240 dyn·s·cm⁻⁵ (≥3 Wood

Units). In further embodiments, prior to initiating treatment with selexipag, SAPH patients have a pulmonary vascular resistance (PVR)≥about 320 dyn·s·cm⁻⁵ (≥4 Wood Units). In other embodiments, the patient has a pulmonary vascular resistance (PVR)≥about 240 dyn·s·cm⁻⁵within about 90 days of initiating the administration of selexipag. In yet further embodiments, the patient has a pulmonary vascular resistance (PVR)≥about 320 dyn·s·cm⁻⁵ within about 90 days of initiating the administration of selexipag. In further embodiments, the PVR is at least about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, or about 600 dyn·s·cm⁻⁵. In other embodiments, the PVR is about 240 to about 600, about 240 to about 550, about 240 to about 500, about 240 to about 450, about 240 to about 400, about 240 to about 350, about 240 to about 300, about 260 to about 600, about 260 to about 550, about 260 to about 500, about 260 to about 550, about 260 to about 500, about 260 to about 450, about 260 to about 400, about 260 to about 350, about 260 to about 300, about 280 to about 600, about 280 to about 600, about 280 to about 550, about 280 to about 500, about 280 to about 500, about 280 to about 450, about 280 to about 400, about 280 to about 350, about 300 to about 600, about 300 to about 550, about 300 to about 500, about 300 to about 450, about 350 to about 600, about 320 to about 550, about 320 to about 500, about 320 to about 450, about 320 to about 600, about 350 to about 550, about 350 to about 500, about 350 to about 450, about 350 to about 400, about 400 to about 600, about 400 to about 550, about 400 to about 500, about 400 to about 450, about 450 to about 600, about 450 to about 550, about 450 to about 500, about 500 to about 600, about 500 to about 550, or about 550 to about 600 dyn·s·cm⁻⁵ or greater. As described herein, PVR is assessed by right heart catherization (RHC).

The methods described herein further reduce the patient's mean pulmonary arterial pressure (mPAP) relative to a patient population at the same or similar level of disease diagnosis that is not receiving treatment with selexipag (placebo). In some embodiments, prior to initiating treatment with selexipag, the SAPH patient has a mPAP of about 25 mmHg or greater (i.e., ≥about 25 mmHg) at rest. In other embodiments, within about 90 days of initiating the administration of the selexipag, the patient has a mPAP of about 25 mmHg or greater (i.e., ≥about 25 mmHg) at rest. In some aspects, the patients have a mPAP at rest of about 25, about 26, about 27, about 28, about 29, about 30, about 31, about 32, about 33, about 34, about 35, about 36, about 37, about 38, about 39, about 40, about 41, about 42, about 43, about 44, or about 45 mmHg at rest. In further embodiments, the patients have a mPAP at rest of about 25 to about 45, about 25 to about 40, about 25 to about 35, about 25 to about 30, about 30 to about 45, about 30 to about 40, about 30 to about 35, about 35 to about 45, about 35 to about 40, about 40 to about 45 mmHg at rest. In other embodiments, the patients have a mPAP at rest of about 25 mmHg to less than about 45 mmHg.

In some embodiments, prior to initiating treatment with selexipag, the SAPH patient has a PAWP of less than about 15 mmHg. In other embodiments, within about 90 days of initiating the administration of the selexipag, the patient has a PAWP of less than about 15 mmHg. In some aspects, patients have a PAWP of less than about 15, about 14, about 13, about 12, about 11, about 10, about 9, about 8, about 7, about 6, about 5, about 4, about 3, about 2, or about 1 mmHg. In other embodiments, patients have a PAWP of about 1 to about 15, about 1 to about 10, about 1 to about 5, about 5 to about 15, about 5 to about 10, or about 10 to about 15 mmHg.

In some embodiments, prior to initiating treatment with selexipag, the patient has a LVEDP of less than about 15 mmHg. In further embodiments, within about 90 days of initiating the administration of the selexipag, the patient has a LVEDP of less than about 15 mmHg. In some aspects, patients have a LVEDP of less than about 15, about 14, about 13, about 12, about 11, about 10, about 9, about 8, about 7, about 6, about 5, about 4, about 3, about 2, or about 1 mmHg. In other embodiments, patients have a LVEDP of about 1 to about 15, about 1 to about 10, about 1 to about 5, about 5 to about 15, about 5 to about 10, or about 10 to about 15 mmHg.

In further embodiments, prior to initiating treatment with selexipag, the patient is in World Health Organization (WHO) functional class II, III, or IV. As known to those skilled in the art, the WHO classifies pulmonary hypertension into functional classes I-IV. See, Table A. In some embodiments, the patient is in WHO functional class II. In other embodiments, the patient is in a WHO functional class III. In further embodiments, the patient is in WHO functional class IV.

TABLE A
WHO Functional Classification of Pulmonary Hypertension

| | |
|---|---|
| Class I | Patients with pulmonary hypertension but without resulting limitation of physical |
| | activity. Ordinary physical activity does not cause undue dyspnea or fatigue, chest |
| | pain or near syncope. |
| Class II | Patients with pulmonary hypertension resulting in slight limitation of physical |
| | activity. They are comfortable at rest. Ordinary physical activity causes undue |
| | dyspnea or fatigue, chest pain or near syncope. |
| Class III | Patients with pulmonary hypertension resulting in marked limitation of physical |
| | activity. They are comfortable at rest. Less than ordinary activity causes undue |
| | dyspnea or fatigue, chest pain or near syncope. |
| Class IV | Patients with pulmonary hypertension with inability to carry out any physical |
| | activity without symptoms. These patients manifest signs of right heart failure. |
| | Dyspnea and/or fatigue may even be present at rest. Discomfort is increased by |
| | any physical activity. |

It is desirable that the SAPH patient is in stable condition prior to initiating treatment with selexipag. The term “stable condition” as used herein refers to a patient having one or more physical abilities. For example, in some embodiments, the patient is able to perform a 6-minute walk test (6MWT) if the patient is in WHO functional class IV prior to initiating the administration of selexipag. The phrase “6-minute walk test” as used herein refers to a non-encouraged test that measures the distance walked in 6 minutes (6MWD). Guidelines on the execution of the 6MWT are described in the American Thoracic Society Committee on Proficiency Standards for Clinical Pulmonary Function Laboratories. ATS statement: guidelines for the six-minute walk test. Am. J. Respir. Crit. Care. Med. 2002; 166:111-117, which is hereby incorporated by reference. In other embodiments, the patient has a 6-minute walk distance (6MWD) between 50 m and 450 m prior to initiating the administration of the selexipag. In further embodiments, the patient does not have left heart failure prior to initiating the administration of selexipag. In yet other embodiments, the patient does not have Child-Pugh class C liver disease prior to initiating the administration of the selexipag. In still further embodiments, the patient does not have a systolic blood pressure of <90 mmHg prior to initiating the administration of the selexipag.

The patient typically is not receiving treatment for pulmonary hypertension prior to initiating the administration of the selexipag or is receiving stable treatment for pulmonary hypertension for at least 90 days, prior to initiating the administration of the selexipag. In some aspects, the patient is not receiving treatment for pulmonary hypertension prior to initiating the administration of the selexipag. In other aspects, the patient is receiving stable treatment for pulmonary hypertension for at least 90 days prior to initiating the administration of the selexipag. The term “stable treatment for pulmonary hypertension” as used herein refers to a treatment for pulmonary hypertension that is prescribed by a physician and the absence of any substantial changes to any prescribed medications. In some embodiments, the stable treatment comprises riociguat, a phosphodiesterase type-5 inhibitor, or an endothelin receptor antagonist, or combinations thereof. In other embodiments, the stable treatment comprises riociguat. In further embodiments, the stable treatment comprises a phosphodiesterase type-5 inhibitor, such as sildenafil or tadalafil. In yet other embodiments, the stable treatment comprises an endothelin receptor antagonist, such as macitentan, ambrisentan, or bosentan.

The patient is also, typically, not receiving treatment for sarcoidosis for at least about 90 days, or is receiving stable treatment for sarcoidosis for at least about 30 days, prior to initiating the administration of the selexipag. In some aspects, the patient is not receiving treatment for sarcoidosis for at least about 90 days prior to initiating the administration of the selexipag. In other aspects, the patient is not receiving a new sarcoidosis specific therapy for at least about 90 days prior to initiating the administration of the selexipag. In other aspects, the patient is receiving stable treatment for sarcoidosis for at least about 30 days, prior to initiating the administration of the selexipag. The term “treatment for sarcoidosis” as used herein refers to a medication in the art that is administered by a physician for treating sarcoidosis. In some aspects, the treatment of sarcoidosis comprises an anti-inflammatory medication. In other aspects, the treatment of sarcoidosis comprises agents such as infliximab, methotrexate, azathioprine, hydroxychloroquine, leflunomide, or steroids.

The SAPH patient, optionally, also is not being treated with a prostacyclin, a prostacyclin analogue, or a prostacyclin receptor agonist. In some aspects, the patient is not being treated with a prostacyclin about 90 days prior to initiating the administration of the selexipag. In other aspects, the patient is not being treated with a prostacyclin analogue about 90 days prior to initiating the administration of the selexipag. In further aspects, the patient is not being treated with a prostacyclin receptor agonist about 90 days prior to initiating the administration of the selexipag.

The methods include administering a therapeutically effective amount of the selexipag. The term “therapeutically effective amount” as used herein, means that amount of active compound or pharmaceutical agent that elicits the biological or medicinal response in a human that is being sought by a researcher, medical doctor, or other clinician, which includes alleviation of the symptoms of the disease or disorder being treated. The selexipag may be administered once daily, twice daily, or thrice daily to achieve the therapeutically effective amount. In some aspects, the selexipag is administered once daily. In other aspects, the selexipag is administered twice daily. In further aspects, the selexipag is administered thrice daily. In yet other aspects, the selexipag is administered twice daily to achieve the therapeutically effective amount.

The selexipag is administered at a starting dose and is increased to determine an individual maximum tolerated dose (iMTD). The term “iMTD” as used herein refers to the maximum amount of selexipag that may be administered to a patient per day, without resulting in adverse physical and/or pharmacological effects. Thus, the iMTD is typically evaluated for each patient on an individual basis. In some aspects, the starting dose of selexipag is the same as the iMTD. In further aspects, the starting dose of selexipag is lower than the iMTD.

The starting dose or the iMTD, on a daily basis, is at least about 10 μg. In some embodiments, the starting dose or the iMTD, on a daily basis, is at least about 100, about 200, about 300, about 400, about 500, about 600, about 700, about 800, about 900, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, about 3100, about 3200, about 3300, about 3400, or about 3500 μg. The daily dose may be administered once daily, twice daily, or thrice daily, preferably twice daily.

Desirably, the iMTD does not exceed about 1600 μg twice daily, i.e., 3200 μg per day. In some embodiments, the iMTD, twice daily, is about 100 to about 3500 μg, about 200 to about 3200, about 200 to about 3000, about 200 to about 2800, about 200 to about 2600, about 200 to about 2400, about 200 to about 2200, about 200 to about 2000, about 200 to about 1800, about 200 to about 1600, about 200 to about 1400, about 200 to about 1200, about 200 to about 1000, about 200 to about 800, about 200 to about 600, about 200 to about 400, about 400 to about 3200, about 400 to about 3000, about 400 to about 2800, about 400 to about 2600, about 400 to about 2400, about 400 to about 2200, about 400 to about 2000, about 400 to about 1800, about 400 to about 1600, about 400 to about 1400, about 400 to about 1200, about 400 to about 1000, about 400 to about 800, about 400 to about 600, about 600 to about 3200, about 600 to about 3000, about 600 to about 2800, about 600 to about 2600, about 600 to about 2400, about 600 to about 2200, about 600 to about 2000, about 600 to about 1800, about 600 to about 1600, about 600 to about 1400, about 600 to about 1200, about 600 to about 1000, about 600 to about 800, about 800 to about 3200, about 800 to about 3000, about 800 to about 2800, about 800 to about 2600, about 800 to about 2400, about 800 to about 2200, about 800 to about 2000, about 800 to about 1800, about 800 to about 1600, about 800 to about 1400, about 800 to about 1200, about 800 to about 1000, about 1000 to about 3200, about 1000 to about 3000, about 1000 to about 2800, about 1000 to about 2600, about 1000 to about 2400, about 1000 to about 2200, about 1000 to about 2000, about 1000 to about 1800, about 1000 to about 1600, about 1000 to about 1400, about 1000 to about 1200, about 1200 to about 3200, about 1200 to about 3000, about 1200 to about 2800, about 1200 to about 2600, about 1200 to about 2400, about 1200 to about 2200, about 1200 to about 2000, about 1200 to about 1800, about 1200 to about 1600, about 1200 to about 1400, about 1400 to about 3200, about 1400 to about 3000, about 1400 to about 2800, about 1400 to about 2600, about 1400 to about 2400, about 1400 to about 2200, about 1400 to about 2000, about 1400 to about 1800, about 1400 to about 1600, about 1600 to about 3200, about 1600 to about 3000, about 1600 to about 2800, about 1600 to about 2600, about 1600 to about 2400, about 1600 to about 2200, about 1600 to about 2000, about 1600 to about 1800, about 1800 to about 3200, about 1800 to about 3000, about 1800 to about 2800, about 1800 to about 2600, about 1800 to about 2400, about 1800 to about 2200, about 1800 to about 2000, about 2000 to about 3200, about 2000 to about 3000, about 2000 to about 2800, about 2000 to about 2600, about 2000 to about 2400, about 2000 to about 2200, about 2200 to about 3200, about 2200 to about 3000, about 2200 to about 2800, about 2200 to about 2600, about 2200 to about 2400, about 2400 to about 3200, about 2400 to about 3000, about 2400 to about 2800, about 2400 to about 2600, about 2600 to about 3200, about 2600 to about 3000, about 2600 to about 2800, about 2800 to about 3200, about 2800 to about 3000, or about 3000 to about 3200 μg. In further embodiments, the iMTD, on a twice daily basis, is about 200 to about 1600 μg.

Desirably, the starting dose is increased in a dose adjustment phase. This “dose-adjustment phase” may be determined by one skilled in the art. Typically, the dose-adjustment phase is about 6 weeks, about 8 weeks, about 10 weeks, about 12 weeks, about 14 weeks, about 16 weeks, about 18 weeks, about 20 weeks, about 22 weeks, about 25 weeks, about 30 weeks, about 35 weeks, about 40 weeks, about 45 weeks, or about 50 weeks. In some embodiments, the dose-adjustment phase is at least about 6 weeks, and more typically from about 8 weeks to about 12 weeks.

During the dose adjustment period, the selexipag dose is increased as determined by one skilled in the art until the iMTD is determined. Typically, the iMTD may be determined within about 8 weeks. In some embodiments, the selexipag dose is increased daily. In other embodiments, the selexipag dose is increased weekly. In further embodiments, the selexipag dose is increased monthly. Preferably, the selexipag dose is increased weekly (based on a 7-day week). When increased weekly, the next selexipag dose may be administered on the same day each week or within 1 day within the scheduled dosing day.

For example, if the selexipag dose is administered on a Monday, the next dose of selexipag may be administered on Sunday, Monday, or Tuesday of the following week. When increased monthly, the selexipag dose may be administered of the same day each month or within 3 days of the next scheduled dosing day. For example, if the selexipag dose in administered on January 7^{th}, the next dose of selexipag may be administered on February 4^{th}, 5^{th}, 6^{th}, 7^{th}, 8^{th}, 9^{th}, or 10^{th}.

Following the dose-adjustment phase, the patient's iMTD is maintained during a maintenance phase. The length of this maintenance phase may be determined by one skilled in the art and is, typically, at least about 14 weeks, and may continue as long as the patient is in need of therapy. In some embodiments, the maintenance phase is about 14, about 16, about 18, about 20, about 22, about 24, about 26, about 28, about 30, about 32, about 34, about 36, about 38, about 40, about 42, about 44, about 46, about 48, about 50, or about 52 weeks. In other embodiments, the maintenance phase is at least about 14 weeks. In further embodiments, the maintenance phase is at least about 26 weeks. In yet other embodiments, the maintenance phase is at least about 1 year, about 2 years, about 3 years, about 4 years, about 5 years, about 10 years, about 20 years, or longer depending on the patient's need for therapy.

Additional dose increases may occur following the maintenance phase as determined by those skilled in the art. However, any such increases desirably do not exceed 1600 μg twice daily.

The methods discussed herein increase the time until first incidence of one or more events selected from all cause death, non-planned pulmonary hypertension related hospitalization, increase in WHO functional class, lung transplantation, atrial balloon septostomy, initiation of parenteral or new class of PH-specific therapy for clinical worsening, deterioration by at least 15% from baseline in exercise capacity as measured by the 6MVVD, or symptoms of right heart failure relative to a patient population at the same or similar level of disease diagnosis that is not receiving treatment with selexipag (placebo). In some embodiments, the methods increase the time until all cause death. In other embodiments, the methods increase the time until non-planned pulmonary hypertension hospitalization. In further embodiments, the methods increase the time until increase in WHO functional class. In still other embodiments, the methods increase the time until deterioration by at least about 15% from baseline in exercise capacity as measured by the 6MVVD as defined herein. In yet further embodiments, the methods increase the time until symptoms of right heart failure relative to a patient population at the same or similar level of disease diagnosis that is not receiving treatment with selexipag. In other embodiments, the methods increase the time until first incidence of lung transplantation. In further embodiments, the methods increase the time until first incidence of atrial balloon septostomy. In yet other embodiments, the methods increase the time until first incidence of initiation of parenteral or new class of PH-specific therapy for clinical worsening

In some embodiments, the methods described herein result in an improvement in the patient's daily life physical activity (DLPA), sleep parameters, or a combination thereof, based on actigraphy assessed daily life physical activity. Such DLPA parameters include, without limitation, one or more of the total DLPA (counts/minute), total volume of activity (above sedentary), daily time spent (minutes) in non-sedentary activity, percentage of daily time spent in non-sedentary activity, and moderate to vigorous physical activity (MVPA), time spent in the different activity categories, or a combination thereof. Such sleep parameters include, without limitation, one or more of total sleep time (TST; minutes), wake after sleep onset (WASO; minutes), number of awakenings, and sleep efficiency (%), or a combination thereof.

In further embodiments, the methods may be effective in evaluating the absolute change and change from baseline in the number of PH low risk criteria. Such evaluations may be performed using measurements such as, without limitation, WHO FC, 6MWD, NT-proBNP Cardiac index, or combinations thereof, up to 6 months after treatment initiation.

In other embodiments, the methods may be effective in reducing any corticoid therapy required by the patient prior to initiation of selexipag treatment, oxygen therapy burden required by the patient prior to initiation of selexipag treatment, or a combination thereof in the patient.

In yet further embodiments, the methods described herein are useful in evaluating the effect of selexipag on disease and pathway-related biomarkers, such as plasma and/or serum biomarkers. Such measurements may be based on the change from baseline and association between biomarker levels and clinical response during the first 9 months, e.g., about 39 weeks, after initiation of selexipag.

In still other embodiments, the methods result in an improvement in pulmonary fibrosis, improvement in post-capillary PH, improvement in lung function-FVC, improvement in ECG signs of right-ventricular strain, or combinations thereof.

As used herein, unless otherwise noted, the term “selexipag” refers to 2-{4-[(5,6-diphenylpyrazin-2-yl)(propan-2-yl)amino]butoxy}-N-(methanesulfonyl)acetamide of formula (I).

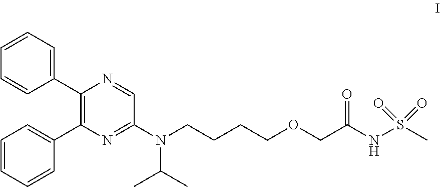 [SMILES: CC(C)N(CCCCOCC(=O)NS(C)(=O)=O)c1cnc(-c2ccccc2)c(-c2ccccc2)n1]

As used herein, “selexipag” also refers to amorphous or crystalline forms of selexipag, such as polymorphs thereof. In some embodiments, the selexipag is a crystalline form, such as a polymorph. In other embodiments, the selexipag is an amorphous form. In other embodiments, the selexipag is the Form I as described in U.S. Pat. Nos. 8,791,122 and 9,284,280, Form II as described in U.S. Pat. No. 9,340,516, or Form III as described in U.S. Pat. No. 9,440,931, all of which are incorporated by reference herein. The crystallinity may be determined by those skilled in the art using one or more techniques such as, e.g., single crystal x-ray diffraction, powder x-ray diffraction, differential scanning calorimetry, melting point, among others.

“Selexipag” as used herein includes anhydrous or hydrates thereof. In certain embodiments, the selexipag is an anhydrous form. In other embodiments, the selexipag is a hydrate thereof.

“Selexipag” as used herein further refers to solvates thereof. Such solvates include a molecule of a solvent bound through intermolecular forces or chemical bonds to one or more locations of the selexipag molecule.

The term “selexipag” may also include pharmaceutically acceptable salts thereof, which may readily be selected by those skilled in the art. A “pharmaceutically acceptable salt” is intended to mean a salt of selexipag that is non-toxic, biologically tolerable, or otherwise biologically suitable for administration to the subject. See, e.g., Berge, “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002, which are incorporated herein by reference. Selexipag can be used in the form of a free base or acid, but can also be used after forming into a pharmaceutically acceptable salt by a known method. When the selexipag is basic, examples of “salt” include salts of inorganic acids such as hydrochloric acid, sulfuric acid, nitric acid, phosphoric acid, hydrofluoric acid and hydrobromic acid, and salts of organic acids such as acetic acid, tartaric acid, lactic acid, citric acid, fumaric acid, maleic acid, succinic acid, methanesulfonic acid, ethanesulfonic acid, benzenesulfonic acid, p-toluenesulfonic acid, naphthalenesulfonic acid and camphorsulfonic acid. When the selexipag is acidic, examples of “salt” include alkali metal salts such as sodium salt and potassium salt, and alkali earth metal salts such as calcium salt.

Geometrical isomers (Z form and E form) of selexipag or mixtures thereof are also contemplated.

Selexipag is commercially available as understood to those skilled in the art. See, e.g., U.S. Pat. No. 7,205,302, which is incorporated by reference herein. For example, selexipag is available as Uptravi® and also is known as ACT-293987, NS-304, or JNJ-6786049. Selexipag is an agonist of the prostacyclin receptor and may be prepared according to the process as disclosed in U.S. Pat. No. 7,205,302.

The present invention also contemplates the administration of selexipag metabolites. Desirably, the selexipag metabolite is metabolically active compound. Thus, in certain embodiments, the selexipag metabolite is of formula M1. M1 is also known under the code name ACT-333679 or MRE-269.

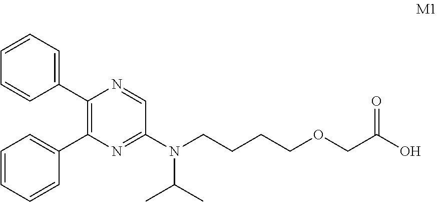 [SMILES: CC(C)N(CCCCOCC(=O)O)c1cnc(-c2ccccc2)c(-c2ccccc2)n1]

As used herein, unless otherwise noted, the terms “treating”, “treatment” and the like, shall include the management and care of a patient for the purpose of combating a disease, condition, or disorder. The terms “treating” and “treatment” also include the administration of the compounds or pharmaceutical compositions as described herein to (a) alleviate one or more symptoms or complications of the disease, condition or disorder; (b) prevent the onset of one or more symptoms or complications of the disease, condition or disorder; and/or (c) eliminate one or more symptoms or complications of the disease, condition, or disorder.

As used herein, unless otherwise noted, the terms “preventing”, “prevention” and the like, shall include (a) reducing the frequency of one or more symptoms; (b) reducing the severity of one or more symptoms; (c) delaying, slowing or avoiding of the development of additional symptoms; and/or (d) slowing, or avoiding the development of the disorder or condition to a later stage or more serious form.

One skilled in the art will recognize that, wherein the present disclosure is directed to methods of prevention, a patient in need of thereof shall include any patient or patient who has experienced or exhibited at least one symptom of the disorder, disease or condition to be prevented. Further, a patient in need thereof may additionally be a patient who has not exhibited any symptoms of the disorder, disease or condition to be prevented, but who has been deemed by a physician, clinician or other medical profession to be at risk of developing said disorder, disease or condition. For example, the patient may be deemed at risk of developing a disorder, disease or condition (and therefore in need of prevention or preventive treatment) as a consequence of the patient's medical history, including, but not limited to, family history, pre-disposition, co-existing (comorbid) disorders or conditions, genetic testing, and the like.

The terms “subject” and “patient” are interchangeably used herein to refer to a human, who has been the object of treatment, observation or experiment. Preferably, the patient has experienced and/or exhibited at least one symptom of the disease or disorder to be treated and/or prevented.

In the methods described herein, the therapeutically effective amount of selexipag is safe. As used herein, unless otherwise noted, the term “safe” shall mean without undue adverse side effects (such as toxicity, irritation, or allergic response), commensurate with a reasonable benefit/risk ratio when used in the manner of this invention.

### Formulations/Compositions

Pharmaceutical compositions containing selexipag as the active ingredient can be prepared by intimately mixing the compound or compounds with a pharmaceutical carrier according to conventional pharmaceutical compounding techniques. As used herein, the terms “composition” and “formulation” are used interchangeably and encompass a product comprising the specified ingredients in the specified amounts, as well as any product, such as a pharmaceutical product, which results, directly or indirectly, from combinations of the specified ingredients in the specified amounts. A summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H.A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

Selexipag may be administered to a patient neat or in a mixture with a pharmaceutically acceptable non-toxic inert carrier, for example, as a pharmaceutical composition containing the compound at a level of 0.1% to 99.5 wt %, preferably 0.5% to 90%, based on the total weight of the composition. As a carrier, one or more of auxiliary agents for formulations such as solid, semi-solid and liquid diluent, filler and other auxiliary agents for drug formulations may be used. It is desirable that a pharmaceutical composition is administered as a unit dosage form. The pharmaceutical composition can be administered into tissue, or intravenously, orally, topically (percutaneously) or rectally. It is a matter of course that a dosage form suitable for any of the administration modes described above is employed. For example, oral administration is preferable.

The pharmaceutical compositions may be administered by a number of routes as determined by those skilled in the art. Preferably, the pharmaceutical compositions are administered by route that is suitable for selexipag. In some embodiments, the pharmaceutical compositions are administered orally, parenterally, or any combination thereof. In other embodiments, the pharmaceutical compositions are administered orally. In further embodiments, the pharmaceutical compositions are administered parenterally.

The pharmaceutical compositions may administered in a form suitable for the selected route of administration. Thus, the pharmaceutical compositions may be administered as suspensions, elixirs, solutions, powders, pills such as capsules, tablets, or caplets, pastilles, granules, syrups, thin films, lozenges, sprays, pastes, or injections. In some embodiments, the pharmaceutical compositions are administered as injections such as intradermal injections, subcutaneous injections, intramuscular injections, intraosseous injections, intraperitoneal injections, or intravenous injections. In other embodiments, the pharmaceutical compositions are administered as suspensions, elixirs, solutions, powders, pills such as capsules (hard or soft), tablets, or caplets, pastilles, granules, syrups, thin films, lozenges, sprays, or pastes. The pills may be formulated for swallowing, chewable, sublingual use, or buccal use, or may be effervescent to be dissolved or dispersed in water prior to administration. In some embodiments, the pharmaceutical product comprises a pill, tablet, powder, sterile parenteral solution, or liquid spray. Desirably, the pharmaceutical product comprises a tablet.

The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Thus, for parenteral administration, the pharmaceutical composition or pharmaceutical product is a sterile, parenteral solution. Injectable suspensions or solutions may also be prepared utilizing aqueous carriers along with appropriate additives.

To prepare such pharmaceutical compositions, selexipag, as the active ingredient, is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above.

The pharmaceutical compositions herein will contain, per unit dosage unit, e.g., tablet, capsule, powder, injection, suppository, teaspoonful and the like, preferably a tablet, of at least about 100, about 200, about 300, about 400, about 500, about 600, about 700, about 800, about 900, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, about 3100, about 3200, about 3300, about 3400, or about 3500 μg. In some embodiments, the pharmaceutical composition comprises about 100 to about 3500 μg, about 200 to about 3200, about 200 to about 3000, about 200 to about 2800, about 200 to about 2600, about 200 to about 2400, about 200 to about 2200, about 200 to about 2000, about 200 to about 1800, about 200 to about 1600, about 200 to about 1400, about 200 to about 1200, about 200 to about 1000, about 200 to about 800, about 200 to about 600, about 200 to about 400, about 400 to about 3200, about 400 to about 3000, about 400 to about 2800, about 400 to about 2600, about 400 to about 2400, about 400 to about 2200, about 400 to about 2000, about 400 to about 1800, about 400 to about 1600, about 400 to about 1400, about 400 to about 1200, about 400 to about 1000, about 400 to about 800, about 400 to about 600, about 600 to about 3200, about 600 to about 3000, about 600 to about 2800, about 600 to about 2600, about 600 to about 2400, about 600 to about 2200, about 600 to about 2000, about 600 to about 1800, about 600 to about 1600, about 600 to about 1400, about 600 to about 1200, about 600 to about 1000, about 600 to about 800, about 800 to about 3200, about 800 to about 3000, about 800 to about 2800, about 800 to about 2600, about 800 to about 2400, about 800 to about 2200, about 800 to about 2000, about 800 to about 1800, about 800 to about 1600, about 800 to about 1400, about 800 to about 1200, about 800 to about 1000, about 1000 to about 3200, about 1000 to about 3000, about 1000 to about 2800, about 1000 to about 2600, about 1000 to about 2400, about 1000 to about 2200, about 1000 to about 2000, about 1000 to about 1800, about 1000 to about 1600, about 1000 to about 1400, about 1000 to about 1200, about 1200 to about 3200, about 1200 to about 3000, about 1200 to about 2800, about 1200 to about 2600, about 1200 to about 2400, about 1200 to about 2200, about 1200 to about 2000, about 1200 to about 1800, about 1200 to about 1600, about 1200 to about 1400, about 1400 to about 3200, about 1400 to about 3000, about 1400 to about 2800, about 1400 to about 2600, about 1400 to about 2400, about 1400 to about 2200, about 1400 to about 2000, about 1400 to about 1800, about 1400 to about 1600, about 1600 to about 3200, about 1600 to about 3000, about 1600 to about 2800, about 1600 to about 2600, about 1600 to about 2400, about 1600 to about 2200, about 1600 to about 2000, about 1600 to about 1800, about 1800 to about 3200, about 1800 to about 3000, about 1800 to about 2800, about 1800 to about 2600, about 1800 to about 2400, about 1800 to about 2200, about 1800 to about 2000, about 2000 to about 3200, about 2000 to about 3000, about 2000 to about 2800, about 2000 to about 2600, about 2000 to about 2400, about 2000 to about 2200, about 2200 to about 3200, about 2200 to about 3000, about 2200 to about 2800, about 2200 to about 2600, about 2200 to about 2400, about 2400 to about 3200, about 2400 to about 3000, about 2400 to about 2800, about 2400 to about 2600, about 2600 to about 3200, about 2600 to about 3000, about 2600 to about 2800, about 2800 to about 3200, about 2800 to about 3000, or about 3000 to about 3200 μg of selexipag. In further embodiments, the pharmaceutical composition comprises about 200 to about 1600 μg of selexipag. In yet other embodiments, the pharmaceutical composition comprises about 200 μg of selexipag. In still further embodiments, the pharmaceutical composition comprises about 400 μg of selexipag. In other embodiments, the pharmaceutical composition comprises about 800 μg of selexipag. In further embodiments, the pharmaceutical composition comprises about 1000 μg of selexipag. In still other embodiments, the pharmaceutical composition comprises about 1200 μg of selexipag. In yet further embodiments, the pharmaceutical composition comprises about 1400 μg of selexipag. In other embodiments, the pharmaceutical composition comprises about 1600 μg of selexipag. The dosages, however, may be varied depending upon the requirement of the patients, the severity of the condition being treated and the compound being employed.

Preferably the pharmaceutical compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. For preparing solid compositions such as tablets, the principal active ingredient (e.g., selexipag) is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present disclosure, or a pharmaceutically acceptable salt thereof. In certain embodiments, two active ingredients can be formulated together, e.g., in a bi-layer tablet formulation. When referring to these preformulation compositions as homogeneous, it is meant that the active ingredients are dispersed evenly throughout the composition so that the composition may be readily subdivided into equally effective dosage forms such as tablets, pills and capsules. The tablets or pills of the composition can be coated or otherwise compounded to provide a dosage form affording the advantage of prolonged action. For example, the tablet or pill can comprise an inner dosage and an outer dosage component, the latter being in the form of an envelope over the former.

The liquid forms in which the compositions of the present disclosure may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The methods described herein may also be carried out using a pharmaceutical composition comprising selexipag and a pharmaceutically acceptable carrier. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, selexipag may be administered in a single daily dose, or the total daily dosage may be administered in divided doses of two, three or four times daily.

For instance, for oral administration in the form of a tablet or capsule, the active drug component (e.g., selexipag) can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare pharmaceutical compositions of the present disclosure, selexipag, as the active ingredient, may be intimately admixed with a pharmaceutical carrier according to conventional pharmaceutical compounding techniques, which carrier may take a wide variety of forms depending of the form of preparation desired for administration (e.g., oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain, the disclosure of which is hereby incorporated by reference.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc., the disclosures of which are hereby incorporated by reference.

The following abbreviations may be used herein.

Abbreviations

| | |
|---|---|
| 6MWD | 6-minute walk distance |
| 6MWT | 6-minute walk test |
| ABG | Arterial Blood Gas |
| AE | adverse event |
| ANCOVA | analysis of covariance |
| ATS | American Thoracic Society |
| BDI | Borg Dyspnea Index |
| Bid | twice daily |
| BP | blood pressure |
| cardiac output | CO |
| CGI-C | Clinician Global impression of Change |
| CGI-S | Clinician Global impression of Severity |
| CHD | coronary heart disease |
| CI | confidence interval |
| CIn | cardiac index |
| Clin(C)RO | clinician-reported outcome |
| CPI | composite physiological index |
| CRF | case report form(s) (paper or electronic as |
| | appropriate for this study) |
| CV | coefficient of variation |
| CYP | cytochrome P450 |
| DB | double-blind |
| DBP | diastolic blood pressure |
| D_{LCO} | diffusing capacity for carbon monoxide |
| dPAP | diastolic pulmonary artery pressure |
| ECG | Electrocardiogram |
| eCRF | electronic Case Report Form |
| EDBT | End-of-Double-Blinded Treatment |
| eGFR | estimated glomerular filtration rate |
| EOLT | End-of-Open-Label Treatment |
| EOMOP | End-of-Main-Observation Period |
| EOS | End-of-Study |
| ERA | endothelin receptor antagonists |
| FAS | Full Analysis Set |
| FC | functional class |
| FEV(1) | forced expiratory volume (in 1 second) |
| FSH | follicle stimulating hormone |
| FVC | forced vital capacity |
| GM | geometric mean |
| HAS | HRCT Analysis Set |
| HR | heart rate |
| HRCT | High-resolution computed tomography |
| HRT | hormonal replacement therapy |
| HS | Hemodynamic Set |
| IA | interim analysis |
| IC | intermittent claudication |
| ICD | implantable cardioverter defibrillator |
| ICF | Informed Consent Form |
| ICH | International Conference on Harmonisation |
| MTD | individual maximum tolerated dose |
| IP | Prostacyclin |
| ISSG | independent, unblinded statistical support group |
| King's (K)SQ | King's sarcoidosis questionnaire |
| LHF | left heart failure |
| LVEDP | left ventricular end-diastolic pressure |
| LVEF | left ventricular ejection fraction |
| MAS | Cardiac MRI Analysis Set |
| MedDRA | Medical Dictionary for Regulatory Activities |
| mPAP | mean pulmonary artery pressure |
| mRAP | mean right atrial pressure |
| MRI | magnetic resonance imaging |
| NT-proBNP | N-terminal pro b-type natriuretic peptide |
| O | Optional |
| OL | open-label |
| PAH | pulmonary arterial hypertension |
| PAH-SYMPACT ™ | Pulmonary Arterial Hypertension-Symptoms |
| | and Impact |
| PAP | pulmonary artery pressure |
| PAWP | pulmonary artery wedge pressure |
| PDE5i | phosphodiesterase type-5 inhibitor |
| PGA-S | Patient Global Assessment of Severity |
| PH | pulmonary hypertension |
| PPS | Per-Protocol Analysis Set |
| PRO | patient-reported outcome |
| PVOD | pulmonary veno-occlusive disease |
| PVR | pulmonary vascular resistance |
| qd | once daily |
| QoL | Quality of Life |
| RBC | red blood cell |
| RHC | right heart catheterization |
| RHF | right heart failure |
| RV | right ventricular |
| SAE | serious adverse event |
| SAP | Statistical Analysis Plan |
| SAPH | sarcoidosis-associated PH |
| SAS | Safety Analysis Set |
| SBP | systolic blood pressure |
| SCR | Screened Analysis Set |
| SF-12 | 12-Item Short Form Health Survey |
| SIS | Selexipag Initiated Set |
| sPAP | systolic pulmonary artery pressure |
| SpO₂ | peripheral capillary oxygen saturation |
| SSc | systemic sclerosis |
| SvO₂ | venous oxygen saturation |
| TC | telephone call |
| TLC | total lung capacity |
| TTCW | time to clinical worsening |
| WHO | World Health Organization |
| WOCP, WOCBP | Women of childbearing potential |
| β-hCG | B-gonadotropin |

Aspects

Aspect 1. A method for treating sarcoidosis-associated pulmonary hypertension, comprising administering to a patient in need thereof a therapeutically effective amount of selexipag.

Aspect 2. A method for treating sarcoidosis-associated pulmonary hypertension in a patient in need thereof, comprising (a) determining if the patient has sarcoidosis-associated pulmonary hypertension; and (b) if the patient has sarcoidosis-associated pulmonary hypertension, administering a therapeutically effective amount of selexipag.

Aspect 3. The method of Aspect 1 or 2, wherein within about 90 days of initiating the administration of the selexipag, the patient has a pulmonary vascular resistance (PVR)≥about 240 dyn·s·cm-5 (≥3 Wood Units), a mean pulmonary arterial pressure (mPAP)≥about 25 mmHg, and a pulmonary arterial wedge pressure (PAWP)≤about 15 mmHg or a left ventricular end diastolic pressure (LVEDP)≤about 15 mmHg.

Aspect 4. The method of any one of the preceding Aspects, wherein, prior to initiating the administration of the selexipag, the patient is in World Health Organization functional class II, III, or IV.

Aspect 5. The method of Aspect 4, wherein the patient is in stable condition and able to perform a 6-minute walk test (6MWT) if the patient is in World Health Organization functional class IV.

Aspect 6. The method of any one of the preceding Aspects, wherein the patient is not receiving treatment for pulmonary hypertension, or is receiving stable treatment for pulmonary hypertension for at least about 90 days, prior to initiating the administration of the selexipag.

Aspect 7. The method of Aspect 6, wherein the stable treatment for pulmonary hypertension comprises riociguat, a phosphodiesterase type-5 inhibitor, or an endothelin receptor antagonist.

Aspect 8. The method of any one of the preceding Aspects, wherein the patient is not receiving treatment for sarcoidosis for at least about 90 days, or is receiving stable treatment for sarcoidosis for at least about 30 days, prior to initiating the administration of the selexipag.

Aspect 9. The method of Aspect 8, wherein the treatment for sarcoidosis comprises anti-inflammatory treatment.

Aspect 10. The method of any one of the preceding Aspects, wherein the patient has a forced vital capacity (FVC)≥50% and a forced expiratory volume (in 1 second) (FEV1)≥about 30% prior to initiating the administration of the selexipag.

Aspect 11. The method of any one of the preceding Aspects, wherein the patient has a 6-minute walk distance (6MWD) between about 50 m and about 450 m prior to initiating the administration of the selexipag.

Aspect 12. The method of any one of the preceding Aspects, wherein, prior to initiating the administration of the selexipag, the patient does not have left heart failure.

Aspect 13. The method of any one of the preceding Aspects, wherein the patient is not being treated with a prostacyclin, a prostacyclin analogue, or a prostacyclin receptor agonist about 90 days prior to initiating the administration of the selexipag.

Aspect 14. The method of any one of the preceding Aspects, wherein the patient does not have Child-Pugh class C liver disease prior to initiating the administration of the selexipag.

Aspect 15. The method of any one of the preceding Aspects, wherein the patient does not have a systolic blood pressure of <about 90 mmHg prior to initiating the administration of the selexipag.

Aspect 16. The method of any one of the preceding Aspects, wherein the selexipag is administered at a starting dose and is increased to determine an individual maximum tolerated dose (iMTD).

Aspect 17. The method of Aspect 16, wherein the starting dose is about 200 μg twice daily.

Aspect 18. The method of Aspect 16 or 17, wherein the iMTD is from about 200 μg to about 1600 μg twice daily.

Aspect 19. The method of any one of Aspects 16-18, wherein the starting dose is increased in twice-daily increments of about 200 μg until the iMTD is determined in a dose adjustment phase.

Aspect 20. The method of Aspect 19, wherein the dose adjustment phase is at least about 12 weeks.

Aspect 21. The method of any one of Aspects 16-20, wherein the iMTD does not exceed about 1600 μg twice daily.

Aspect 22. The method of any one of Aspects 19-21, wherein the iMTD is maintained during a maintenance phase following the dose adjustment phase.

Aspect 23. The method of Aspect 22, wherein the maintenance phase is at least about 14 weeks.

Aspect 24. The method of Aspect 23, wherein the maintenance phase is at least about 26 weeks.

Aspect 25. The method of Aspect 23 or 24, wherein a further dose increase takes place following the maintenance phase, but does not exceed about 1600 μg twice daily.

Aspect 26. The method of any one of the preceding Aspects, wherein the method reduces the patient's PVR relative to a patient at the same level of disease diagnosis that is not receiving treatment with selexipag.

Aspect 27. The method of any one of the preceding Aspects, wherein the method increases the time until first incidence of one or more events selected from all cause death, non-planned pulmonary hypertension hospitalization, increase in WHO functional class, deterioration by at least 15% from baseline in exercise capacity as measured by the 6MWD, or symptoms of right heart failure relative to a patient at the same level of disease diagnosis that is not receiving treatment with selexipag.

Aspect 28. The method of any one of the preceding Aspects wherein the selexipag is administered orally in the form of a tablet.

The following Examples are provided to illustrate some of the concepts described within this disclosure. While the Examples are considered to provide embodiments, they should not be considered to limit the more general embodiments described herein. In the following examples, efforts have been made to ensure accuracy with respect to numbers used (e.g., amounts, temperature, etc.) but some experimental error and deviation should be accounted for.

### EXAMPLES

### Example 1

Selexipag is supplied as round, film-coated tablets in different strengths: 200, 400, 600, 800, 1000, 1200, 1400, and 1600 μg strength.

The compositions are given in Table 1 and Table 2.

TABLE 1
Composition of selexipag film-coated tablets (200-800 μg)

| | Selexipag film-coated tablet | | | |
|---|---|---|---|---|
| | 200 μg | 400 μg | 600 μg | 800 μg |
| Ingredients | Amount (mg) | | | |
| Selexipag | 0.2 | 0.4 | 0.6 | 0.8 |
| Mannitol | 72.6 | 72.4 | 72.2 | 72.0 |
| Maize starch | 48.0 | | | |
| Low substituted | 6.8 | | | |
| hydroxypropylcellulose | | | | |
| Hydroxypropylcellulose | 5.4 | | | |
| Magnesium stearate | 2.0 | | | |
| Core tablet weight | 135.0 | | | |
| Hypromellose | 3.8000 | | | |
| Propylene glycol | 0.7000 | | | |
| Titanium dioxide | 0.4505 | 0.3500 | 0.4880 | 0.1500 |
| Iron oxide red | — | 0.1500 | 0.0050 | — |
| Iron oxide black | — | — | 0.0070 | 0.1000 |
| Iron oxide yellow | 0.0495 | — | — | 0.2500 |

| | |
|---|---|
| Carnauba wax | 0.040 |
| Coating weight | 5.0 |
| Total weight of film-coated | 140.0 mg |
| tablet | |

TABLE 2
Composition of selexipag film-coated tablets (1000-1600 μg)

| | Selexipag film-coated tablet | | | |
|---|---|---|---|---|
| | 1000 μg | 1200 μg | 1400 μg | 1600 μg |
| Ingredients | Amount (mg) | | | |
| Selexipag | 1.0 | 1.2 | 1.4 | 1.6 |
| Mannitol | 71.8 | 71.6 | 71.4 | 71.2 |
| Maize starch | 48.0 | | | |
| Low substituted | 6.8 | | | |
| hydroxypropylcellulose | | | | |
| Hydroxypropylcellulose | 5.4 | | | |
| Magnesium stearate | 2.0 | | | |
| Core tablet weight | 135.0 | | | |
| Hypromellose | 3.8000 | | | |
| Propylene glycol | 0.7000 | | | |
| Titanium dioxide | 0.4310 | 0.4640 | 0.2500 | 0.1500 |
| Iron oxide red | 0.0195 | 0.0150 | — | 0.1250 |
| Iron oxide black | — | 0.0210 mg | — | 0.1250 |
| Iron oxide yellow | 0.0495 | — | 0.2500 | 0.1000 |

| | |
|---|---|
| Carnauba wax | 0.040 |
| Coating weight | 5.0 |
| Total weight of film-coated | 140.0 mg |
| tablet | |

### Example 2

The overall benefit/risk assessment for this clinical study is considered acceptable because there is an unmet medical need for therapeutic options for patients with SAPH.

Safety will be closely monitored throughout the study:
In general, safety evaluations (including but not limited to thyroid function tests, blood pressure monitoring, hematology laboratory tests) will be performed at scheduled visits during the study, as indicated in Tables 4 and 5.
Any clinically significant abnormalities (including those persisting at the end of the study/early withdrawal) will be followed until resolution or until a clinically stable condition is reached.

Participants will discontinue study intervention for the reasons discussed herein.

A. Objectives and Endpoints

Table 3 provides the objectives and endpoints of the methods described herein:

TABLE 3

| | |
|---|---|
| Objectives | Endpoints |

Primary

| | |
|---|---|
| To assess the effect of selexipag | PVR at peak concentration ^{a} at Week 20 expressed as |
| versus placebo on PVR in | percent of the baseline value. |
| participants with SAPH at Week 20. | |

Secondary

| | |
|---|---|
| To evaluate the effect of selexipag | TTCW (defined according to definition by the CHMP^{b}) |
| versus placebo on TTCW. | up to Week 52 defined as at least one of the following |
| | components: |
| | All-cause death |
| | Non-planned PH-related hospitalization |
| | Increase in WHO FC |
| | Deterioration by at least 15% from baseline in |
| | exercise capacity as measured by the 6MWD |
| | Signs or symptoms of RHF defined as a reported AE |
| | with one of the following preferred terms: |
| | “pulmonary hypertension”, “right ventricular |
| | failure”, “right ventricular dysfunction” and “acute |
| | right ventricular failure”. |
| To evaluate the effect of selexipag | Longitudinal trend of 6MWD up to Week 52. |
| versus placebo on exercise capacity. | Proportion of participants with oxygen desaturation |
| | post 6MWT up to Week 52 (identified by decrease in |
| | SpO₂ by at least 5% from pre-6MWT). |
| To evaluate the effect of selexipag | Proportion of participants with improvement, worsening |
| versus placebo on WHO FC. | and no change from baseline in WHO FC up to Week 52. |
| To evaluate the effect of selexipag | Proportion of participants with all-cause death or PH- |
| versus placebo on death or PH- | related hospitalization up to Week 52. |
| related hospitalizations | Rate of all-cause death or PH-related hospitalization |
| | up to Week 52 - the rate will be expressed as per 100- |
| | participant years, calculated by dividing the total |
| | number of all-case death or hospitalizations related to |
| | PH-worsening by the cumulative exposure up to Week |
| | 52 of all participants in each invention group |
| To evaluate the effect of selexipag | Change from baseline up to Week 52 in SF-12 scores. |
| versus placebo on PROs assessed by | Change from baseline up to Week 52 in KSQ scores. |
| the SF-12, KSQ, PAH-SYMPACT ™, | Change from baseline up to Week 39 in PAH- |
| and PGA-S | SYMPACT scores. |
| | Change from baseline up to Week 52 in PGA-S |
| | scores. |
| To evaluate the effect of selexipag | Change from baseline up to Week 52 in CGI-S scores. |
| versus placebo on CROs assessed by | Change from baseline up to Week 52 in CGI-C scores. |
| the CGI-S and CGI-C | |

Safety

| | |
|---|---|
| To assess the overall safety of | Intervention-emergent AEs. |
| selexipag | Intervention-emergent prostacyclin-associated AEs. |
| | SAEs up to EOS. |
| | AEs leading to premature discontinuation of study |
| | treatment. |
| | Treatment-emergent AEs of special interest (e.g., |
| | hypotension, anemia, hyperthyroidism). |
| | Change in vital signs (systolic and diastolic arterial |
| | blood pressure and pulse rate) and body weight from |
| | baseline to all assessed timepoints during the study. |
| | Treatment-emergent marked laboratory abnormalities. |
| | Change from baseline in supplemental oxygen rate. |

Other/Exploratory

| | |
|---|---|
| To explore the effect of selexipag on | Number and change from baseline in the number of low- |
| the number of PAH low-risk criteria | risk criteria based on WHO FC, 6MWD, NT-proBNP, |
| | and CIn. |
| To explore the effect of selexipag on | Change from baseline in clinical risk in pulmonary |
| clinical risk in pulmonary sarcoidosis | sarcoidosis based on CPI. |
| as assessed by the CPI | |
| To explore the effect of selexipag on | Change from baseline in % pulmonary fibrosis and shift |
| pulmonary fibrosis in a subset of | in radiographic stage assessed by HRCT. |
| participants | |
| To explore the effect of selexipag on | Change from baseline in the RV hemodynamics and |
| RV function and morphology in a | morphology as assessed by cardiac MRI. |
| subset of participants | |
| To explore the change in | Change from baseline in other hemodynamic variables |
| hemodynamic variables other than | (including cardiac output [CO], CIn, mRAP). |
| PVR | |
| To explore the change in pulmonary | Change from baseline in TLC, FVC and D_{LCO}. |
| function | |
| To explore the long-term effect of | Change from baseline in 6MWD, BDI, oxygen |
| selexipag on clinical variables | saturation, and WHO FC. |
| To explore the effect of selexipag on | Change from baseline in NT-proBNP serum levels. |
| NT-proBNP | |
| To explore the effect of selexipag on | Change from baseline in serum biomarkers and |
| disease and pathway-related serum | associations between serum biomarker levels and |
| biomarkers | clinical response and baseline characteristics. |
| To explore the effect of selexipag on | Medical resource utilization |
| medical resource utilization | |

^{a} 2 to 5 hours post-dose.
^{b}The investigation of a composite endpoint that reflects the time to clinical worsening has been encouraged by the European Medicines Agency [EMEA 2008].

B. Study Design

1. Overall Design

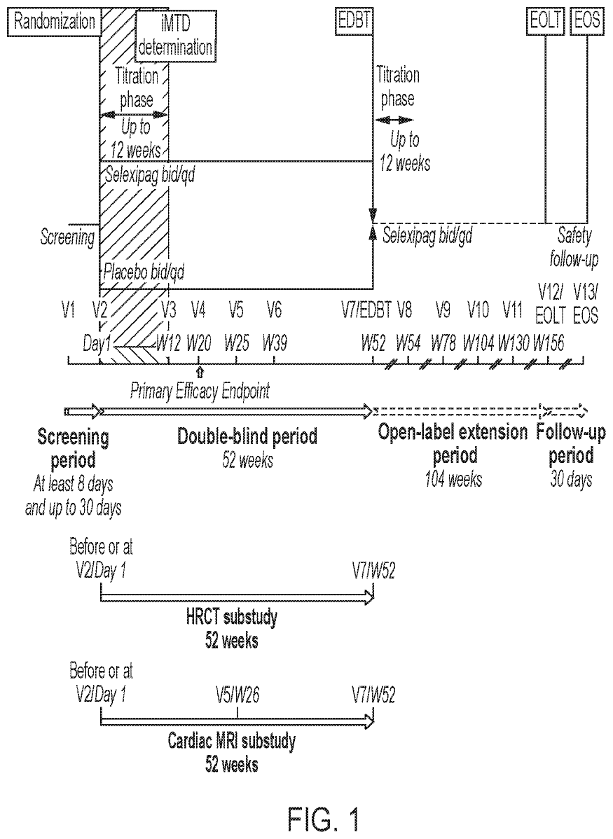

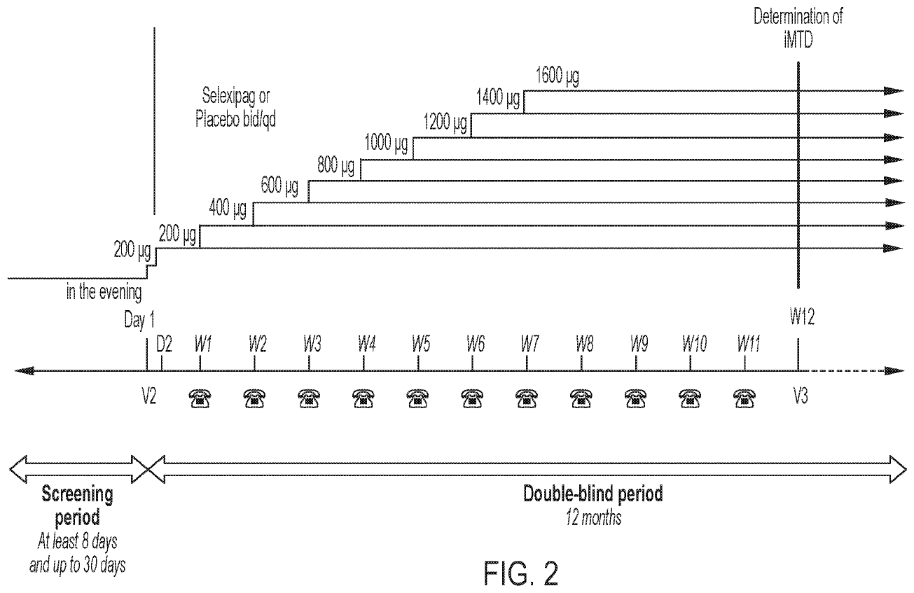

This is a prospective, randomized, double-blind (DB), group-sequential, placebo-controlled, multicenter, interventional 52-week study with a 104-week OL extension period in men and women ≥18 and ≤75 years of age with SAPH. Study intervention will be up-titrated to alloweach participant to reach their iMTD, in the range of 200 μg to 1600 μg twice daily (bid). For participants with moderate hepatic impairment (Child-Pugh B) or who are concomitantly taking (a) moderate cytochrome P450 (CYP)2C8 inhibitor(s) the dosing frequency is once daily (qd). Diagrams of the study design and titration are provided in FIGS. 1 and 2, respectively. In these figures, dosing frequency, in general, will be twice daily (bid), except for participant with moderated hepatic impairment (Child-Pugh B) or who are concomitantly taking (a) moderate CYP2C8 inhibitor(s), for whom the dosing frequency is to be reduced to qd. During the double-blind period, these participants must take the stud intervention in the morning, hence the first dose of study intervention should be taken in the morning of Day 2. During the open-label period, participants who are a qd regimen can take the first does of study intervention at a random timepoint during the day, and study intervention will be administered preferably at the same timepoint thereafter.

Approximately 150 participants will be recruited in 2 sequential cohorts. The first 86 randomized participants will constitute the hemodynamic cohort and will undergo RHC at baseline and at Week 20, in addition to the overall study assessments. The remaining participants will constitute the non-hemodynamic cohort. In contrast to the participants enrolled in the hemodynamic cohort, participants from the non-hemodynamic cohort will not undergo RHC at Week 20; other assessments will be the same for the 2 cohorts. See, for future analysis from all participants. Both cohorts will be combined for evaluation of secondary efficacy endpoints, which do not require hemodynamic assessments. See, Tables 4 and 5.

TABLE 4
Schedule of Activities - Double-Blind Period

| | | | | | | | | | | | Safety | |
|---|---|---|---|---|---|---|---|---|---|---|---|---|
| | | | | | | | | | | Premature | Follow- | |
| Periods | Name | Screening | DOUBLE-BLIND PHASE (52 weeks) | | | | | | | EDBT | up | |
| | | | | Weekly | | | | | | | | U1, 2, 3, |
| Visits | Number | 1 | 2 | TC | 3 | 4 | 5 | 6 | 7 | | | etc. |
| | | | | | | | | | | | Safety | |
| | | | | | | | | | | | follow- | |
| | | | | Titration | | | | | | | up | |
| | | | | Phase | | | | | | Premature | phone | Unscheduled |
| | | | | (W 1, | | | | | | EDBT^{a, Error!} | call^{Error!} | visit^{Error!} |
| | | | | W 2, | | | | | Week | ^{Reference} | ^{Reference} | ^{Reference} |
| | | | Random- | W 3, | Week | Week | Week | Week | 52/ | ^{source not} | ^{source not} | ^{source not} |
| | Name | Screening | ization | etc.) | 12 | 20 | 26 | 39 | EDBT | ^{found.} | ^{found.} | ^{found.} |
| | | | | | | | | | | Within | | |
| | | | | Day 8 | | | | | | 7 days | | |
| | | | | (±3 | | | | | | after last | 30 | |
| | | | | days) and | | | | | | DB dose in | (+5) | |
| | | | | weekly | | | | | | case of DB | days | |
| | | | | (±3 | | | | | | inter- | after | |
| | | | | days) | | | | | | vention | study | |
| | | | | until | Day | Day | Day | Day | Day | discontin- | inter- | |
| | | Day −30 | | Day 78 | 85 | 141 | 183 | 274 | 365 | uation | vention | |
| | | to | | (±3 | (±7 | (±7 | (±7 | (±7 | (±7 | before | discontin- | |
| | Time | Day −8 | Day 1 | days) | days) | days) | days) | days) | days) | Week 52 | uation | Any time |

Screening/Administrative

| | | |
|---|---|---|
| ICF^{Error!} | X | |
| ^{Reference} | | |
| ^{source not} | | |
| ^{found.} | | |
| Optional | X | |
| biomarker ICF | | |
| Demographics | X | |
| and height | | |
| Review medical | X | |
| history | | |
| requirements | | |
| Inclusion/exclusion | X | X |
| criteria | | |
| Prestudy | X | X |
| therapy | | |
| Serum | X | |
| pregnancy test | | |
| (WOCBP)^{Error!} | | |
| ^{Reference} | | |
| ^{source not} | | |
| ^{found., Error!} | | |
| ^{Reference} | | |
| ^{source not} | | |
| ^{found.} | | |

| | | | | |
|---|---|---|---|---|
| Urine | X | monthly (±7 days) | X | X |
| pregnancy test | | | | |
| (WOCBP)^{Error!} | | | | |
| ^{Reference} | | | | |
| ^{source not} | | | | |
| ^{found.} | | | | |

Study Intervention Administration

| | | | | | | | | |
|---|---|---|---|---|---|---|---|---|
| Randomization | X | | | | | | | |
| DB study | X | | X | X | X | X | X | X |
| intervention | | | | | | | | |
| dispensing/ | | | | | | | | |
| return^{Error!} | | | | | | | | |
| ^{Reference} | | | | | | | | |
| ^{source not} | | | | | | | | |
| ^{found.} | | | | | | | | |
| DB study | X | X | X | X | X | X | X | |
| intervention | | | | | | | | |
| administration^{Error!} | | | | | | | | |
| ^{Reference} | | | | | | | | |
| ^{source not} | | | | | | | | |
| ^{found.} | | | | | | | | |

Efficacy Evaluations

| | | | | | | | | | |
|---|---|---|---|---|---|---|---|---|---|
| RHC^{Error!} | X^{Error!} | | | X^{Error!} | | | | | |
| ^{Reference} | ^{Reference} | | | ^{Reference} | | | | | |
| ^{source not} | ^{source not} | | | ^{source not} | | | | | |
| ^{found.} | ^{found.} | | | ^{found.} | | | | | |
| 6MWT^{Error!} | X | X | X | X | X | X | X | X | X^{Error!} |
| ^{Reference} | | | | | | | | | ^{Reference} |
| ^{source not} | | | | | | | | | ^{source not} |
| ^{found., m}/ | | | | | | | | | ^{found.} |
| BDI^{Error!} | | | | | | | | | |
| ^{Reference} | | | | | | | | | |
| ^{source not} | | | | | | | | | |
| ^{found., m}/ | | | | | | | | | |
| SpO₂^{Error!} | | | | | | | | | |
| ^{Reference} | | | | | | | | | |
| ^{source not} | | | | | | | | | |
| ^{found., m} | | | | | | | | | |
| WHO FC | X | X | X | X | X | X | X | X | X^{Error!} |
| | | | | | | | | | ^{Reference} |
| | | | | | | | | | ^{source not} |
| | | | | | | | | | ^{found.} |
| SF-12^{Error!} | X | X | X | X | X | X | X | X | |
| ^{Reference} | | | | | | | | | |
| ^{source not} | | | | | | | | | |
| ^{found., Error!} | | | | | | | | | |
| ^{Reference} | | | | | | | | | |
| ^{source not} | | | | | | | | | |
| ^{found.} King's | | | | | | | | | |
| gQ^{Error!} | | | | | | | | | |
| ^{Reference} | | | | | | | | | |
| ^{source not} | | | | | | | | | |
| ^{found., Error!} | | | | | | | | | |
| ^{Reference} | | | | | | | | | |
| ^{source not} | | | | | | | | | |
| ^{found.} | | | | | | | | | |
| PGA-S^{Error!} | | | | | | | | | |
| ^{Reference} | | | | | | | | | |
| ^{source not} | | | | | | | | | |
| ^{found., Error!} | | | | | | | | | |
| ^{Reference} | | | | | | | | | |
| ^{source not} | | | | | | | | | |
| ^{found.} | | | | | | | | | |
| PAH- | X | X | X | X | X | X | | | |
| SYMPACT ™^{Error!} | | | | | | | | | |
| ^{Reference} | | | | | | | | | |
| ^{source not} | | | | | | | | | |
| ^{found., Error!} | | | | | | | | | |
| ^{Reference} | | | | | | | | | |
| ^{source not} | | | | | | | | | |
| ^{found.} | | | | | | | | | |
| CGI-S^{Error!} | | X | X | X | X | X | X | X | |
| ^{Reference} | | | | | | | | | |
| ^{source not} | | | | | | | | | |
| ^{found., Error!} | | | | | | | | | |
| ^{Reference} | | | | | | | | | |
| ^{source not} | | | | | | | | | |
| ^{found.} | | | | | | | | | |
| CGI-C^{Error!} | | | X | X | X | X | X | X | |
| ^{Reference} | | | | | | | | | |
| ^{source not} | | | | | | | | | |
| ^{found., Error!} | | | | | | | | | |
| ^{Reference} | | | | | | | | | |
| ^{source not} | | | | | | | | | |
| ^{found.} | | | | | | | | | |
| NT- | | X | X | X | X | X | X | X | |
| proBNP^{Error!} | | | | | | | | | |
| ^{Reference} | | | | | | | | | |
| ^{source not} | | | | | | | | | |
| ^{found.}, ^{Error!} | | | | | | | | | |
| ^{Reference} | | | | | | | | | |
| ^{source not} | | | | | | | | | |
| ^{found.} | | | | | | | | | |
| DL_{CO}^{Error!} | X | | X | | X | | X | X | |
| ^{Reference} | | | | | | | | | |
| ^{source not} | | | | | | | | | |
| ^{found.} | | | | | | | | | |
| Spirometry^{Error!} | X | | X | | X | | X | X | |
| ^{Reference} | | | | | | | | | |
| ^{source not} | | | | | | | | | |
| ^{found.} | | | | | | | | | |

Safety Evaluations

| | | | | | | | | | |
|---|---|---|---|---|---|---|---|---|---|
| Vital signs (BP, | X | X | X | X | X | X | X | X | X^{Error!} |
| HR), weight | | | | | | | | | ^{Reference} |
| | | | | | | | | | ^{source not} |
| | | | | | | | | | ^{found.} |
| Physical | X | X | X | X | X | X | X | X | X^{Error!} |
| examination^{Error!} | | | | | | | | | ^{Reference} |
| ^{Reference} | | | | | | | | | ^{source not} |
| ^{source not} | | | | | | | | | ^{found.} |
| ^{found., Error!} | | | | | | | | | |
| ^{Reference} | | | | | | | | | |
| ^{source not} | | | | | | | | | |
| ^{found.} | | | | | | | | | |
| Local 12-lead | X | | | | | | X | X | |
| ECG (single)^{Error!} | | | | | | | | | |
| ^{Reference} | | | | | | | | | |
| ^{source not} | | | | | | | | | |
| ^{found.} | | | | | | | | | |

Clinical Laboratory Tests

| | | | | | | | | |
|---|---|---|---|---|---|---|---|---|
| Hematology | X | X^{Error!} | X | X | X | X | X | X |
| and | | ^{Reference} | | | | | | |
| clinical | | ^{source not} | | | | | | |
| chemistry^{Error!} | | ^{found.} | | | | | | |
| ^{Reference} | | | | | | | | |
| ^{source not} | | | | | | | | |
| ^{found.} | | | | | | | | |

Biomarkers

| | | | | | |
|---|---|---|---|---|---|
| Serum and | X | X | X | X | X |
| plasma samples | | | | | |
| for exploratory | | | | | |
| biomarker | | | | | |
| research^{Error!} | | | | | |
| ^{Reference} | | | | | |
| ^{source not} | | | | | |
| ^{found., Error!} | | | | | |
| ^{Reference} | | | | | |
| ^{source not} | | | | | |
| ^{found., Error!} | | | | | |
| ^{Reference} | | | | | |
| ^{source not} | | | | | |
| ^{found.} | | | | | |

Medical Resource Utilization

| | | | | | | | | | |
|---|---|---|---|---|---|---|---|---|---|
| Medical | X | X | X | X | X | X | X | X | X |
| resource | | | | | | | | | |
| utilization | | | | | | | | | |

Ongoing Participant Review

| | | | | | | | | | | | |
|---|---|---|---|---|---|---|---|---|---|---|---|
| Concomitant | | | X | X | X | X | X | X | X | X | X |
| Therapy | | | | | | | | | | | |
| SAEs/AEs^{Error!} | X | X | X | X | X | X | X | X | X | X | X |
| ^{Reference} | | | | | | | | | | | |
| ^{source not} | | | | | | | | | | | |
| ^{found.} | | | | | | | | | | | |

Optional Pulmonary Fibrosis Substudy

| | | |
|---|---|---|
| Centrally read | X | X |
| HRCT^{Error!} | | |
| ^{Reference} | | |
| ^{source not} | | |
| ^{found., Error!} | | |
| ^{Reference} | | |
| ^{source not} | | |
| ^{found.} | | |

Optional Cardiac MRI Substudy

| | | | |
|---|---|---|---|
| Centrally read | X | X | X |
| cardiac | | | |
| MRI^{Error!} | | | |
| ^{Reference} | | | |
| ^{source not} | | | |
| ^{found., Error!} | | | |
| ^{Reference} | | | |
| ^{source not} | | | |
| ^{found.} | | | |

Other

| | | |
|---|---|---|
| Participant | X^{Error!} | X^{Error!} |
| experience | ^{Reference} | ^{Reference} |
| survey^{Error!} | ^{source not} | ^{source not} |
| ^{Reference} | ^{found.} | ^{found.} |
| ^{source not} | | |
| ^{found., Error!} | | |
| ^{Reference} | | |
| ^{source not} | | |
| ^{found.} | | |

^{a}For participants who prematurely discontinue study intervention for any reason (except for withdrawal from the study).
^{b} If the premature EDBT visit treatment falls within the visit window of any other scheduled visit, these visits can be combined, and assessments will not be repeated.
^{c} Unscheduled visits may be performed at any time during the DB period. Assessments (other than reporting of AE/SAE and concomitant medication) are to be discretionally performed. During the DB period, an unscheduled visit must be performed.
^{d} Must be signed before first study-related activity.
^{e} A serum pregnancy test must be performed at Screening or in case of a positive urine pregnancy test.
^{f} Transferred electronically by an external service provider.
^{g} Assessment not collected in eCRF.
^{h} Only applicable in case the participant did not prematurely discontinue the DB intervention, in which case no study drug will be dispensed as soon as the premature EDBT visit is performed.
^{I}Only applicable in case the participant did not prematurely discontinue the DB study intervention. In case of bid dosing, first dose to be taken in the evening. In case of qd dosing, the first dose of study intervention should be taken in the morning of Day 2. Includes study intervention up-titration.
^{j} If the participant prematurely discontinues DB intervention, this is not mandatory and should be performed discretionally.
^{k} A historical RHC is allowed taking into account the restrictions. If no historical results are available, an RHC must be performed during the Screening period only if all other inclusion criteria are met and none of the exclusion criteria is met.
^{l} Only for participants in the hemodynamic cohort and must be performed after the post-dose 6MWT. Assessment to be performed within 2 to 5 hours post-dose.
^{m}To be performed within 2 to 5 hours post-dose. In the hemodynamic cohort, this should be performed prior to the RHC (if applicable).
ⁿ In case an unscheduled visit is performed, this assessment must be performed.
^{o} If the results for the screening blood samples from the central laboratory are not available in time for randomization of the participant, an additional blood sample may be drawn to verify eligibility based on a local laboratory test.
^{p} Optional assessment.
^{q} All AE and SAEs will be reported throughout the study from signing of the ICF onwards until the EOS visit or date of last contact.
^{r} Either on the EDBT visit (only for participants who are not enrolled into the OL period) or on the premature EDBT visit, whichever occurs last.

TABLE 5
Schedule of Activities - Open-label Period

| Periods | Name | Transition | Open-Label Phase (24 month) | | | | Safety |
|---|---|---|---|---|---|---|---|
| Visits | Number | 7 | Weekly TC | 8 | 9, 10, 11 | 12 | Follow-Up |
| | Name | Week 52/ | Titration | Week | Week 78, 104 | Week 156/ | EOS |
| | | EDBT | Phase (W53, | 64 | and 130 | EOLT | (Telephone call) |
| | | | W54, W55, | | | | |
| | | | etc.) | | | | |
| | Time | Day 365 | Day 372 | Day | Day 547, 729 | Day 1093 | 30 (+5) days |
| | | (±7 days) | (±3 days) and | 449 | and 911 | (±7 days) or for | after study |
| | | | weekly | (±7 days) | (±7 days) | premature | intervention |
| | | | (±3 days) until | | | EOLT within 7 | completion/ |
| | | | Day 442 | | | days after last | discontinuation |
| | | | (±3 days) | | | OL dose | |

Administrative

| | | |
|---|---|---|
| Urine pregnancy test | monthly (±7 days) | X |
| (WOCBP)^{Error! Reference} | | |
| ^{source not found., Error!} | | |
| ^{Reference source not found.} | | |

Study Intervention Administration

| | | | | | |
|---|---|---|---|---|---|
| OL study intervention | X | | X | X | X |
| dispensing/return | | | | | |
| OL study intervention | X | X | X | X | X |
| administration^{Error!} | | | | | |
| ^{Reference source not found.} | | | | | |

Efficacy Evaluation

| | | |
|---|---|---|
| 6MWT/BDI/SpO₂ | X | X |
| WHO FC | X | X |
| DL_{CO} | X | X |
| Spirometry | X | X |

Safety Evaluations

| | | | |
|---|---|---|---|
| Vital signs (BP, HR), | X | X | X |
| Weight | | | |
| Physical | X | X | X |
| examination^{Error!} | | | |
| ^{Reference source not found.} | | | |

Clinical laboratory tests

| | | | |
|---|---|---|---|
| Hematology and | X | X | X |
| clinical chemistry^{Error!} | | | |
| ^{Reference source not found.} | | | |

Medical Resource Utilization

| | | | | | |
|---|---|---|---|---|---|
| Medical resource | X | X | X | X | X |
| utilization | | | | | |

Ongoing Participants Review

| | | | | | |
|---|---|---|---|---|---|
| Concomitant therapy | X | X | X | X | X |
| SAEs/AFs^{Error! Reference} | X | X | X | X | X |
| ^{source not found.} | | | | | |

Other

| | |
|---|---|
| Participant experience | X |
| survey^{Error! Reference} | |
| ^{source not found., Error!} | |
| ^{Reference source not found.} | |

^{a} Assessment not collected in eCRF.
^{b} A serum pregnancy test must be performed at Screening or in case of a positive urine pregnancy test.
^{c} Only applicable if the participant did not prematurely discontinue the OL study intervention. In case of bid dosing, first OL dose to be taken on the evening of Visit 7. In case of qd dosing, participants can take the first dose of study intervention at a random timepoint during the day, and study intervention will be administered preferably at the same timepoint thereafter. Includes study intervention up-titration.
^{d} Transferred electronically by an external service provider.
^{e} Optional assessment.
^{f} All AE and SAEs will be reported throughout the study from signing of the ICF onwards until the EOS visit or date of last contact. Unscheduled visits may be performed at any time during the OL treatment period. Assessments are performed discretionally.

Participants will be randomized in a 1:1 ratio to receive either selexipag or placebo during the DB period of 52 weeks. Randomization will be stratified by PH-specific therapies at baseline (yes vs no). Participants who do not discontinue study intervention during the DB period until Visit 7 (Week 52) will enter the OL period and will receive selexipag for an additional 104 weeks. Participants who prematurely discontinue the DB study intervention cannot enter the OL period. Information on the required procedures to follow if participants discontinue the DB study intervention or the OL study intervention prematurely is provided herein.

The study starts with the first ICF signed by the first participant and ends with the last safety follow-up TC or visit of the last participant. The study comprises the following periods:

(a) An 8- to 30-day Screening period: starts with the signature of the ICF and ends with the participant's randomization at Visit 2, Day 1.

(b) 2 intervention periods:
A 52-week DB period which starts with the administration of the first dose of DB study intervention in the evening of the day of randomization (Visit 2, Day 1) and with a titration phase of up to 12 weeks. It ends on the day of the last dose of DB study intervention with the EDBT visit (Visit 7).
A 104-week, single-arm, OL period extending intervention for participants who completed the 52 weeks of DB intervention. The period starts with the first dose of the OL study intervention (selexipag) in the evening of the day of the last dose of DB study intervention, i.e., the EDBT visit (Visit 7) and ends with the EOLT visit (Visit 12). All participants entering the OL period will have to up-titrate OL selexipag, irrespective of the previous intervention assignment

(c) A safety follow-up period which starts on the day after the last dose of study intervention and ends 30 (+5) days later with the safety follow-up TC.

The duration of individual participation will be approximately 3 years. The visit schedule and protocol-mandated procedures are performed according to Tables 4 and 5 for the respective study periods. The efficacy assessments include, but are not limited to, RHC at Week 20 (only for the hemodynamic cohort), assessment of exercise capacity, dyspnea, and WHO FC, measurement of the NT-proBNP levels, recording PROs and CROs, and pulmonary function tests.

Safety and tolerability will be evaluated throughout the study from signing of the ICF onwards until the EOS visit or date of last contact. Safety and tolerability assessments include review of concomitant medications and AEs, clinical laboratory tests, 12-lead electrocardiogram (ECG), vital signs, physical examination, and pregnancy testing.

Plasma and serum samples will be collected.

Medical resource utilization data will be collected. There are 4 Analysis Timepoints:

Analysis Timepoint 1 (futility for PVR): When approximately 34 participants in the hemodynamic cohort have completed the Week 20 RHC assessment (or discontinued prematurely).

Analysis Timepoint 2 (final analysis for PVR and interim analysis [IA] for TTCW): When all participants of the hemodynamic cohort (pre-planned 86 participants) have completed the Week 20 RHC and the Week 26 assessments (or discontinued prematurely).
Analysis Timepoint 3 (final analysis for TTCW, end of DB): When all participants have completed the DB period (or discontinued prematurely). At this timepoint, the final analysis of the TTCW endpoint will be conducted. Data for all remaining endpoints will also be analyzed. After the interim database lock for the DB period is done, the study team will be unblinded.
Analysis Timepoint 4 (end of OL): When all participants have completed the OL period (24 months) and safety follow-up or discontinued earlier. At this timepoint, all available data will be analyzed.

2. Scientific Rationale for Study Design

Rationale for the Use of Placebo/Randomization/Stratification/Blinding/Cohorts

A placebo-controlled study conducted in a randomized and DB fashion will be used to evaluate the efficacy of selexipag and will allow to establish the frequency and magnitude of changes in hemodynamic and clinical endpoints that may occur in the absence of active intervention. The use of a placebo control will also allow proper evaluation of any safety related events or abnormalities and disease progression observed during the study and to differentiate between events potentially related to the use of selexipag vs those related to the underlying disease.

The use of background therapy with PH-specific therapies (excluding prostanoids, prostacyclin analogues and non-prostanoid IP receptor agonists) is allowed. In addition, treatment escalation of PH-specific therapies is allowed following the Week 26 visit (Visit 5). Stable treatment for sarcoidosis is also allowed.

Randomization will be used to minimize bias in the assignment of participants to intervention groups, to increase the likelihood that known and unknown participant attributes (e.g., demographic and baseline characteristics) are evenly balanced across intervention groups, and to enhance the validity of statistical comparisons across intervention groups.

Blinded intervention will be used to reduce potential bias during data collection and evaluation of clinical endpoints. Randomization will be stratified by PH-specific therapies at baseline (yes vs no) to enhance the validity of statistical comparisons across intervention groups and to account for a potentially different treatment effect in participants already receiving PH-specific therapies compared to treatment-naive participants.

To limit to a minimum the number of participants in the hemodynamic cohort, participants will be recruited in 2 sequential cohorts. The first approximately 86 participants constitute the hemodynamic cohort and, in addition to the overall study assessments, will undergo an RHC at Week 20. The remaining participants will constitute the non-hemodynamic cohort. Both cohorts are combined for evaluation of secondary efficacy endpoints, which do not require a post-baseline hemodynamic assessment.

Rationale for the Duration of the Study

Participants will receive DB selexipag or placebo for 52 weeks. PVR and other hemodynamic endpoints will be assessed at Week 20 by RHC. TTCW and other secondary and exploratory endpoints, e.g., rate of hospitalization related to PH-worsening or death and exercise capacity, will be assessed up to Week 52.

In order to comply with the intent-to-treat principle, participants who prematurely discontinue the DB study intervention will be followed up until Week 52.

An OL extension period aims to collect long-term efficacy, safety and tolerability information on selexipag and the survival status of participants with SAPH, and to provide access to selexipag for participants who have completed the DB intervention.

Rationale for Right Heart Catherization

Precapillary PH is characterized by an increased resistance to blood flow in the pulmonary vasculature quantified by PVR. PVR is determined by RHC. In addition, a historical RHC is allowed for all participants in the non-hemodynamic cohort and for participants in the hemodynamic cohort, if the historical RHC was performed in accordance with the guidance provided herein.

Study-Specific Ethical Design Considerations: This study is being conducted to evaluate the efficacy and safety of selexipag in participants with SAPH. The total blood volume to be collected from the population over the study period (see Table 7) is considered to be acceptable based upon WHO recommendations.

3. Justification for Dose

Study intervention will be up-titrated to allow each participant to reach their iMTD, in the range of 200 μg to 1600 μg bid; or in the range of 200 μg to 1600 μg qd for participants with moderate hepatic impairment (Child-Pugh B) or who are concomitantly taking moderate CYP2C8 inhibitor(s). Depending on the iMTD, a single dose of study intervention will consist of 1 to 8 tablets (200 μg to 1600 μg). See, Example 1 for the selexipag formulation for each dose.

4. End-of-Study Definition

EOS Visit: For participants who completed the DB study intervention and completed or prematurely discontinued study intervention during the OL period for any reason (except for withdrawal from the study as defined herein), the EOS visit corresponds to the EOS TC in the OL period. For participants who prematurely discontinue DB study intervention for any reason (except for withdrawal from the study as defined herein) but completed the DB period, the EOS visit corresponds to the last visit/TC, which is either Visit 7 (Week 52) or the safety follow-up TC, whichever occurs last, in the DB period. See, Tables 4 and 5.

5. Participant Completion

Double-blind Period: A participant, whether on study intervention or not, will be considered to have completed the DB period of the study if he or she has completed the visits up to and including Visit 7 (Week 52) or the safety follow-up phone call, whichever occurs last. Participants who prematurely discontinue the DB study intervention cannot enter the OL period.

Open-label Period: A participant, whether on study intervention or not, will be considered to have completed the OL period of the study if he or she has completed the EOS safety follow-up phone call.

Study: A participant will be considered to have completed the study if he or she did not prematurely withdraw from the study, and has completed an EOS visit. Participants who do not complete the DB or OL period as defined above and/or withdraw from the study, will not be considered to have completed the study.

Study Completion: The End-of-Study (EOS) is considered as the last visit or EOS visit, whichever occurs last, for the last participant in the study.

C. Study Population

1. Inclusion Criteria

Each potential participant must satisfy all of the following criteria to be enrolled in the study:
Male or female.
18 (or the legal age of consent in the jurisdiction in which the study is taking place) to 75 years of age, inclusive, at Screening.
Confirmed diagnosis of sarcoidosis as per ATS criteria.
Sarcoidosis-associated precapillary PH, confirmed by RHC (at rest) within 90 days prior to randomization:

a. PVR≥240 dyn* sec/cm⁵ (≥3.0 Wood units)

b. Mean pulmonary artery pressure (mPAP)≥25 mmHg

c. Pulmonary artery wedge pressure (PAWP)≤15 mm Hg, or if not available or unreliable, a LVEDP≤15 mmHg. A historical RHC is allowed taking into account the restrictions provided herein. If no historical results are available, an RHC must be performed during the Screening period, but only if all other inclusion criteria are met and none of the exclusion criteria is met.
PH severity according to modified WHO FC II-IV at Screening and randomization; participants of WHO FC IV must be in a stable condition and able to perform a 6MWT.
No treatment or stable treatment of PH (i.e., riociguat, PDE5i, ERA) during at least 90 days prior to randomization and the RHC qualifying for enrollment, (i.e., no introduction of new therapies or dose change).
Stable sarcoidosis treatment regimen, i.e., no new specific anti-inflammatory treatment for sarcoidosis for at least 90 days, and stable dose(s) for at least 30 days prior to randomization and the RHC qualifying for enrollment.
6MWD between 50 and 450 m both at Screening and at time of randomization.
Forced vital capacity (FVC)>50% and FEV₁>30% of predicted at Screening. Short-acting-beta-agonists (e.g., salbutamol) and long-acting-beta-agonists must not be taken 6 hours and 24 hours prior to spirometry testing, respectively.
FEV₁/FVC≥60%, or if FEV1/FVC<60% then FEV₁ must be ≥65% of predicted at Screening. Short-acting-beta-agonists (e.g., salbutamol) and long-acting-beta-agonists must not be taken 6 hours and 24 hours prior to spirometry testing, respectively.
For patients enrolled or planned to be enrolled in a cardio-pulmonary rehabilitation program based on exercise training, one of the following must apply:

(a) In the maintenance phase of the program at the time of randomization with no plans to stop the program until Week 26, or

(b) Start of the cardio-pulmonary rehabilitation program based on exercise training is planned after Week 26.
A woman must be:

(a) Not of childbearing potential

(b) Of childbearing potential and
Have a negative highly sensitive serum (β-hCG) at Screening and a negative urine pregnancy test at randomization.
Agree to undertake monthly urine pregnancy tests during the study and up to at least 30 days after study intervention discontinuation.
Practicing an acceptable method of contraception and agree to remain on an acceptable method while receiving study intervention and until 30 days after last dose of study intervention.
   A woman using oral contraceptives must have been using this method for at least 1 month prior to randomization.
   Willing and able to adhere to the lifestyle restrictions specified herein.

2. Exclusion Criteria

Any potential participant who meets any of the following criteria will be excluded from participating in the study:
PH due to left heart disease (PAWP >15 mmHg).
PH due to compression of pulmonary arteries and/or pulmonary veins.
History of LHF including cardiomyopathies, cardiac sarcoidosis, with a LVEF <40%.
CHD or unstable angina.
Myocardial infarction within the last 6 months prior to or during Screening.
Decompensated cardiac failure if not under close supervision.
Arrhythmias assessed as severe.
ICD for secondary prevention.
Cerebrovascular events (e.g., transient ischemic attack, stroke) within the last 90 days prior to or during Screening.
Congenital or acquired valvular defects with clinically relevant myocardial function disorders not related to PH.
Overt features of pulmonary veno-occlusive disease (PVOD).
Significant emphysema.
Known and documented severe hepatic impairment (Child-Pugh C).
Severe renal failure (eGFR<30 mL/min/1.73 m² or serum creatinine>2.5 mg/dL) based on central laboratory results from the Screening blood sample.
Treatment with prostacyclin, prostacyclin analogues or IP receptor agonists (i.e., selexipag) during 90 days prior to randomization and/or prior to the RHC qualifying for enrollment, except those given at vasodilator testing during RHC.
Included on a lung transplant list or planned to be included until Visit 5/Week 26.
Known or suspected uncontrolled thyroid disease.
Treatment with moderate inducers of CYP2C8, e.g. rifampicin or strong inhibitors of CYP2C8, e.g., gemfibrozil at or within 14 days prior to randomization.
Change in dose or initiation of new diuretics and/or calcium channel blockers within 1 week prior to RHC qualifying for enrollment.
SBP<90 mmHg at Screening or at randomization.
Known allergies, hypersensitivity, or intolerance to selexipag or its excipients.
Planned or current treatment with another investigational treatment up to 90 days prior to randomization.
Received an investigational intervention or used an invasive investigational medical device within 90 days prior to randomization.
Any condition for which participation would not be in the best interests of the participant (e.g., compromise well-being), or that could prevent, limit, or confound the protocol-specified assessments.
Any acute or chronic impairment that may influence the ability to comply with study requirements such as to perform RHC, a reliable and reproducible 6MWT (e.g., use of walking aids (cane, walker, etc.), or lung function tests.
Employee of the investigator or study site, with direct involvement in the proposed study or other studies under the direction of that investigator or study site, as well as family members of the employees or the investigator.

D. Study Intervention

1. Description of Study Interventions

Double-blind selexipag 200 μg, matching placebo, and OL selexipag 200 μg will be provided.

2. Study Intervention Administration

The tablets are administered orally and should be swallowed whole (i.e., not crushed, split or chewed) with water. Dosing frequency will be bid, except for participants with moderate hepatic impairment (Child-Pugh B) or who are concomitantly taking (a) moderate CYP2C8 inhibitor(s), for whom the dosing frequency is qd. In case of bid dosing, at the beginning of each up-titration step, the first dose is to be taken in the evening. During the DB period and in case of qd dosing, participants must take the study intervention in the morning; hence the first dose of study intervention should be taken in the morning of Day 2. During the OL period, participants who are on a qd regimen can take the first dose of study intervention at a random timepoint during the day, and study intervention will be administered preferably at the same timepoint thereafter. Beginning with the morning of Visit 3, and at each visit thereafter of the DB period, participants must take the morning study intervention dose before any visit-related procedure. Tolerability may improve when study drug is taken with food.

3. Study Intervention Up-Titration

Study intervention will be up-titrated to allow each participant to reach their iMTD, in the range of 200 μg to1600 μg (i.e., 1 to 8 tablets) bid/qd. Dosing frequency will be bid, except for participants with moderate hepatic impairment (Child-Pugh B) or who are concomitantly taking (a) moderate CYP2C8 inhibitor(s), who receive study intervention qd.

(i) Double-Blind Study Intervention Up-Titration

Each participant will start with 1 tablet of DB study intervention, i.e., selexipag (200 μg) or matching placebo, in the evening of Day 1 and will continue with 200 μg bid on Day 2. Participants with moderate hepatic impairment (Child-Pugh B) or who are concomitantly taking (a) moderate CYP2C8 inhibitor(s) will start with 200 μg qd in the morning of Day 2. The dose will be up-titrated in 200 μg bid/qd increments at weekly intervals during scheduled TCs until reaching the iMTD. See, Table 6 and FIG. 2.

TABLE 6
Study Intervention Up-titration Scheme

| Period | Dose regimen | Duration | |
|---|---|---|---|
| First dose | 200 μg | On Day 1 in the evening (pm) | 1 tablet |
| Up-titration | 200 μg bid* | From Day 2 am to Day 8 am** | 1 tablet bid^{§} |
| | 400 μg bid*, ^{§} | *From Day 8 pm to Day 15 am** | 2 tablets bid^{§} |
| | 600 μg bid*, ^{§} | From Day 15 pm to Day 22 am** | 3 tablets bid^{§} |
| | 800 μg bid*, ^{§} | From Day 22 pm to Week 4 am** | 4 tablets bid^{§} |
| | 1000 μg bid*, ^{§} | From Week 4 pm to Week 5 am** | 5 tablets bid^{§} |
| | 1200 μg bid*, ^{§} | From Week 5 pm to Week 6 am** | 6 tablets bid^{§} |
| | 1400 μg bid*, ^{§} | From Week 6 pm to Week 7 am** | 7 tablets bid^{§} |
| | 1600 μg bid*, ^{§} | From Week 7 pm to Week 8 am** | 8 tablets bid^{§} |
| Maintenance | iMTD: | From Week 12 onwards | 1-8 tablets bid^{§} |
| | 200 μg to 1600 μg bid^{§} | | |

am = morning; pm = evening;
*Or the iMTD until Week 12.
**If the dose regimen is not well tolerated or symptoms cannot be fully managed with symptomatic treatment, the duration of the titration step can be prolonged to 2 weeks.
^{§}For participants with moderate hepatic impairment (Child-Pugh B) or who are concomitantly taking (a) moderate CYP2C8 inhibitor(s) the dosing frequency is qd. During the DB period, these participants must take the study intervention in the morning; hence the first dose of study intervention should be taken in the morning of Day 2. During the OL period, these participants can take the first dose of study intervention at a random timepoint during the day, and study intervention will be administered preferably at the same timepoint thereafter.

If the dose regimen is not well tolerated or symptoms cannot be fully managed with symptomatic treatment, the duration of the titration step can be prolonged to 2 weeks. If needed, the dose can be reduced by 200 μg bid/qd. At Week 12 (Visit 3), the bid/qd dose reached for each participant is defined as the iMTD. This dose must be kept stable at least until Week 26 (Visit 5). Any study intervention interruption of 3 days or more will require a new up-titration to avoid tolerability-limiting side effects.

After Week 26, it will be allowed to up-titrate the dose of the participant further if needed (up to the maximum of 1600 μg bid/qd, as applicable) in 200 μg bid/qd increments at scheduled or unscheduled visits. Participants continue receiving study intervention up to Week 52 (Visit 7). Starting with Visit 3 and at all consecutive Visits during the DB period, 6MWT and RHC must be performed within 2 to 5 hours post-dose.

(ii) Open-Label Study Intervention Up-Titration:

All participants entering the OL period will have to up-titrate OL selexipag, irrespective of the previous intervention assignment. The last DB study intervention dose must be taken in the morning before performing the assessments of Visit 7. The OL study intervention up-titration will start with 1 tablet of OL selexipag (200 μg) in the evening of Visit 7 and continues with 200 μg bid on the day after. Participants with moderate hepatic impairment (Child-Pugh B) or who are concomitantly taking (a) moderate CYP2C8 inhibitor(s) will receive the first dose of 200 μg qd at a random timepoint during the day, and study intervention will be administered preferably at the same timepoint thereafter.

The up-titration procedure will be as for the DB study intervention up-titration. The dose will be up-titrated in 200 μg bid/qd increments at weekly or biweekly intervals until reaching the iMTD (see Table 6). Although up-titration intervals can be flexible, the total time to reach the iMTD is expected to be 12 weeks. However, it is allowed to further up-titrate the dose beyond 12 weeks if needed (up to the maximum of 1600 μg bid/qd, as applicable) in 200 μg bid/qd increments at scheduled or unscheduled visits. Open-label selexipag will be taken up to 24 months until the end of OL period at Week 156 (Visit 12).

E. Measures to Minimize Bias: Randomization and Blinding

1. Intervention Allocation

Procedures for Randomization and Stratification: Central randomization will be implemented in this study. Participants will be randomly assigned to 1 of 2 intervention groups based on a computer-generated randomization schedule prepared before the study. The randomization will be balanced by using randomly permuted blocks and will be stratified by PH-specific therapy at baseline (yes vs no).

Blinding: The first 52 weeks from randomization of this study will be performed in a DB fashion.

2. Study Intervention Compliance

Study intervention compliance is based on study intervention accountability. Study intervention compliance will be calculated by site personnel at each visit using the below formula:

$$\frac{( number of tablets dispensed - number of tablets returned )}{\begin{matrix}number of tablets that should \\ have been taken during the period\end{matrix}} ⨯ 100$$

3. Concomitant Therapy

Prestudy therapies administered up to 30 days before first dose of study intervention and any previous therapies for the treatment of PH (ERA, PDE5i, riociguat) and sarcoidosis (e.g., infliximab, methotrexate, azathioprine, hydroxychloroquine, leflunomide, steroids) administered up to 90 days before the baseline RHC and 90 days before randomization, respectively, must be recorded at time of Screening.

Concomitant medications, except those listed below, are allowed. Concomitant therapies must be recorded throughout the study from signing of the ICF onwards until the EOS visit or date of last contact. All therapies (oxygen supplementation, prescription or over-the-counter medications, including vaccines, vitamins, herbal supplements; non-pharmacologic therapies such as electrical stimulation, acupuncture, special diets, exercise regimens) different from the study intervention must be recorded. The following medications and/or therapies are not allowed to be administered concomitantly with study intervention:
Prostacyclin (epoprostenol), prostacyclin analogues (e.g., treprostinil, iloprost, beraprost) or prostacyclin receptor agonists (i.e., selexipag/UPTRAVI®) until study intervention discontinuation.
Strong inhibitors of CYP2C8 (e.g., gemfibrozil) until study intervention discontinuation.
Change in dose or initiation of new diuretics and/or calcium channel blockers within 1 week prior to RHC qualifying for enrollment.
Any other investigational drug up to 30 days after study intervention discontinuation.

Medications and/or therapies allowed to be administered concomitantly with study intervention, include, but are not limited to:
Stable doses of therapies for the treatment of PH (i.e., riociguat, PDE5i, ERA). Change in dose or initiation of new therapies for the treatment of PH are allowed as of Week 26 (Visit 5).
Short-acting-beta-agonists (e.g., salbutamol) and long-acting-beta-agonists, but must not be taken within 6 hours and 24 hours prior to spirometry testing, respectively.
Single administration of prostacyclin or analogues used for acute vasodilator testing during an RHC procedure.
Moderate inhibitors of CYP2C8. Dosing frequency of study intervention must be reduced to qd if a moderate inhibitor of CYP2C8 (e.g., clopidogrel, deferasirox, teriflunomide, leflunomide) is concomitantly administered. Dosing frequency of selexipag should be reverted to bid when co-administration of moderate CYP2C8 inhibitor is stopped.
Sarcoidosis-specific treatment, e.g., infliximab, methotrexate, azathioprine, hydroxychloroquine, leflunomide, steroids. Initiation of new treatment or change in dose is allowed as of Week 26 (Visit 5).
Strong inhibitors of UGT1A3 and UGT2B7 (e.g., valproic acid, probenecid, and fluconazole). The effect of strong inhibitors of UGT1A3 and UGT2B7 on the exposure to selexipag and its active metabolite has not been studied. Caution is required when administering these medicinal products concomitantly with selexipag. A potential pharmacokinetic interaction with strong inhibitors of UGT1A3 and UGT2B7 cannot be excluded.

4. Dose Modification

For participants who are unable to tolerate the protocol-specified dosing scheme, dose adjustments must follow the down-titration instructions.

F. Discontinuation of Study Intervention and Participant

Discontinuation/Withdrawal

1. Discontinuation of Study Intervention

A participant's study intervention must be discontinued if:
For safety or tolerability reasons (e.g., AE) it is in the best interests of the participant to discontinue study intervention.
The participant becomes pregnant.
Hepatic impairment occurs or is suspected. If hepatic impairment is suspected, a clinical assessment of severity (Child-Pugh score) should be performed. If a participant has developed severe hepatic impairment (Child-Pugh C) at any time during the study, the study intervention must be permanently discontinued.
Pulmonary edema due to PVOD occurs. Should signs of pulmonary edema occur, the possibility of associated PVOD should be considered. If confirmed, the study intervention must be discontinued.
Study intervention interruptions exceeding 14 consecutive days.
The participant withdraws consent to receive study intervention.

If a participant discontinues study intervention before the end of the DB period, he/she will continue to perform the visits and assessments as scheduled until Week 52, provided the participant's consent for this limited participation in the study has not been withdrawn. The participant will be asked to return for a premature EDBT visit within 7 days of last intake of DB study intervention and for a safety follow-up phone call 30 (+5) days after the last intake of DB study intervention. If the premature EDBT visit falls within the visit window of any other scheduled visit, these visits can be combined, and assessments will not be repeated. These participants cannot enter the OL period.

If a participant discontinues study intervention before the end of the OL period, he/she will be followed up until 30 (+5) days after OL study intervention discontinuation, provided that the participant's consent for this limited participation in the study has not been withdrawn. The participant will be asked to return for a premature EOLT visit within 7 days of last intake of OL study intervention and to have an EOS safety follow-up phone call 30 (+5) days after the last intake of OL study intervention. At the premature EOLT visit, the assessments described for the regular EOLT visit (Visit 12) are to be performed. See, Tables 4 and 5.

Temporary Discontinuation: Study intervention may be temporarily interrupted in response to an AE, a diagnostic or therapeutic procedure, a laboratory abnormality, or for administrative reasons. Interruptions of study intervention must be kept as short as possible. Any study intervention interruption of 3 days or more will require a new up-titration starting with 1 tablet of study intervention (200 μg) bid/qd. For each participant, the up-titration frequency will be up to the medical judgment of the investigator and based on his/her clinical evaluation of the participants' tolerability of the study drug prior to its interruption. Re-uptitration to a previously reached dose can be done at scheduled or unscheduled phone calls or visits, respectively. Study intervention interruptions exceeding 14 consecutive days must lead to permanent discontinuation of study intervention.

2. Participant Discontinuation/Withdrawal From the Study

A participant will not be automatically withdrawn from the study if they have to discontinue study intervention before the end of the intervention regimen. A participant will be withdrawn from the study for any of the following reasons:
Lost to follow-up.
Withdrawal of consent.
Death.
Participant is poorly compliant with study procedures, visits, and assessments.
It is considered that continued participation in the study would be contrary to the best interests of the participant.
Decision for any reason, including, but not limited to premature termination or suspension of the study.

G. Study Assessments and Procedures

1. Overview

Tables 4 and 5 summarize the frequency and timing of efficacy, safety, biomarker, and medical resource utilization measurements applicable to this study. If it is not possible to complete all assessments on the same day, a visit may extend over more than 1 day within the allowed time window. The following order of assessments is recommended, as applicable:
PROs completed at each site Visit: SF-12, King's SQ, PGA-S
Safety assessments
Pulmonary function tests
WHO FC
6MWT* and oxygen saturation and BDI (pre- and post-6MWT)
Blood samples for hematology and clinical chemistry tests, NT-proBNP, and/or biomarkers
RHC* (applies only to the hemodynamic cohort).

*Assessments to be performed within 2 to 5 hours post-dose.

The maximum total blood volume to be collected during the entire study from each participant will be approximately 172 mL (see Table 7).

TABLE 7
Volume of Blood to be Collected From Each Participant

| | | | | |
|---|---|---|---|---|
| | | | | Approximate |
| | Volume | No. of | No. of | Maximum |
| | per | Samples per | Samples per | Total |
| | Sample | Participant | Participant | Volume of |
| Type of Sample | (mL) | (DB period) | (OL period) | Blood (mL)^{a} |

Safety (including Screening and post-intervention assessments)

| | | | | |
|---|---|---|---|---|
| Hematology | 2 | 7 | 5 | 24 |
| Serum chemistry | 7.5 | 7 | 5 | 90 |
| NT-proBNP | 2.5 | 7 | 0 | 17.5 |
| Serum β-hCG | 2.5 | 1 | 0 | 2.5 |
| pregnancy tests | | | | |
| Exploratory | 4.5 | 4 | 0 | 18 |
| biomarker plasma | | | | |
| sample ^{b} | | | | |
| Exploratory | 5 | 4 | 0 | 20 |
| biomarker serum | | | | |
| sample ^{b} | | | | |

| | |
|---|---|
| Approximate Total ^{d} | 172^{c} |

^{a}Calculated as total number of samples (DB period + OL period) multiplied by amount of blood per sample.
^{b} Optional sample.
^{c}During the entire study the approximate total volume is 170 mL for male participants as no serum β-hCG pregnancy test will be performed for these participants.
^{d} Repeated or unscheduled samples may be taken for safety reasons or technical issues with the samples. n indwelling intravenous cannula may be used for blood sample collection.

2. Demographics and Baseline Characteristics

Demographic and baseline characteristic data to be collected on all randomized participants include: age, sex, race and ethnicity (where local regulations permit), weight and height, date of the initial SAPH diagnosis, WHO FC, and smoking status (never, former, current).

For participants who are naive to PH-specific therapies, the reason(s) for not prescribing ERA/PDE-5 inhibitors/riociguat are collected.

3. Efficacy Assessments

(a) Primary Efficacy Endpoint Assessment

(i) Right heart catheterization

The following hemodynamic variables are to be collected: HR and peripheral systolic and diastolic blood pressure at start of the RHC procedure.

Pulmonary artery wedge pressure (PAWP; alternatively LVEDP), mRAP, systolic/diastolic/mean pulmonary artery pressures (sPAP/dPAP/mPAP), CO, mixed venous oxygen saturation (SvO₂), and PVR are to be collected.

(ii) Baseline RHC: All participants must have a baseline RHC. A historical RHC is allowed, if following criteria are met:
Historical RHC was performed as described herein
It is feasible to perform the Week 20 RHC at the same location and under the same conditions (e.g., same method, same flow rate of oxygen [if applicable], and whenever possible by the same operator) as the historical RHC
Historical RHC was performed:
   Within 90 days prior to randomization,
   At least 90 days after last change in PH-specific therapies (i.e., change in dose or initiation of new class of drugs).
   At least 90 days after starting a new specific anti-inflammatory treatment for sarcoidosis.
   At least 30 days after last change in dose of specific anti-inflammatory treatment for sarcoidosis.

If no historical results meeting the above criteria are available, an RHC must be performed during the Screening period only if all other inclusion criteria are met and none of the exclusion criteria is met.

(iii) Week 20 RHC

At Week 20, an RHC will be done in participants enrolled into the hemodynamic cohort. The RHC must be performed within 2 to 5 hours post-dose, at the same location and under the same conditions (e.g., same method, same flow rate of oxygen [if applicable], and whenever possible by the same operator) as the baseline RHC.

(b) Secondary Efficacy Endpoints Assessments

(i) Clinical Worsening and Hospitalization Related toPH-worsening: The following data are required to derive TTCW as per CHIMP definition: reporting of death, non-planned PH-related hospitalization, WHO FC, 6MWD, AEs and SAEs.

(ii) Exercise Capacity: Measured by the 6MWT. During the DB-period, the 6MWT must be performed within 2 to 5 hours post-dose and prior to the RHC (if applicable). The time of the study intervention morning dose intake will be recorded. For participants who have never performed a 6MWT previously, a training test will be requested before the Screening 6MWTs for inclusion. The walking length should be 25 to 30 m; the exact length should be documented on the 6MWT Corridor Card. If a 6MWT cannot be performed at a scheduled visit beyond Visit 2 or at an unscheduled visit, a reason must be provided (i.e., PH-related or other).

(iii) Oxygen Saturation (Pre- and Post-6MWT): Measured using pulse oximetry (performed as per standard practice at the study site) just before starting and immediately after stopping the 6MWT.

(iv) Dyspnea (Pre- and Post-6MWT): Assessed by the BDI, a scale used to quantify the degree of shortness of breath on a scale from “0” to “10” as described in Borg G. Perceived exertion as an indicator of somatic stress. Scand. J. Rehab. Med. 1970; 2:92-98. Dyspnea will be evaluated by each individual participant immediately before starting and after stopping the 6MWT.

(v) WHO FC: Assessed as described herein and understood in the art.

(vi) NT-proBNP: A blood sample will be drawn.

(vii) Patient-reported Outcomes:
PAH-SYMPACT™ questionnaire: The PAH-SYMPACT™ questionnaire is a PRO instrument that was developedby Actelion Pharmaceuticals Ltd. The PAH-SYMPACT™ should be completed in the evening before bedtime. This questionnaire has been developed and validated for use in PAH patients. The PAH-SYMPACT™ consists of 2 parts:
   The symptom part is completed for the 7 consecutive days following the visit at site (i.e., starting the day following the visit day).
   The impact part is completed once, on the seventh day of the symptoms diary data collection period, together with the symptom part (i.e., in the evening).
SF-12: The SF-12v2® Healthy Survey is a quality of life self-assessment derived from the SF-36v2® (Health Survey ©1996, 2000 by Medical Outcomes Trust and Quality Metric Incorporated). It has 12 items measuring 8 scales, including physical functioning, physical role limitations, bodily pain, general health, vitality, social functioning, emotional role limitations, and mental health. The SF-12 is scored using norm-based scoring in the same way as for the SF-36v2®.
King's Sarcoidosis questionnaire (KSQ): The KSQ is a health status questionnaire developed and validated for patients with sarcoidosis. It is brief, adaptable to individual patients and assesses general and organ-specific health status. The KSQ consists of 5 modules: General health status (10 items), Lung (6 items), Skin (3 items), Eye (7 items), Medications (3 items). The organ-specific modules can be combined with the General health status module to assess overall health status for patients depending on which organs are affected by their disease. KSQ is summarized as a number between 1 and 100, with higher numbers indicating better health.
Patient Global Assessment of Disease Severity (PGA-S): The PGA-S is a 6-point single item self-evaluation scale. Participants will be asked to rate the overall severity of their disease on the day of administration, with responses of none, very mild, mild, moderate, severe or very severe.
Clinician Global Impression of Severity (CGI-S): The CGI-S is a 6-point single-item scale. The overall severity of the participant's disease will be rated on the day of administration with responses of none, very mild, mild, moderate, severe or very severe.
Clinician Global Impression of Change (CGI-C): The CGI-C is a 7-point single-item scale. The overall participant's change of the participant's disease will be rated since baseline with responses of very much better, moderately better, a little better, no change, a little worse, moderately worse or very much worse.

(viii) Other Endpoint Assessments
Pulmonary Function Tests
   Spirometry: Spirometry tests will be conducted according to the ATS/ERS. It is recommended that all spirometry assessments are performed at the same time and under same conditions throughout the study. Participants must refrain from taking short-acting-beta-agonists (e.g., salbutamol) for 6 hours and long-acting-beta-agonists for 24 hours prior to spirometry testing. If taken, the test should be rescheduled. To perform the spirometry test, participants will be rested for a minimum of 5 minutes prior to start. The following variables will be collected at Screening (Visit 1) and subsequent Visits where applicable: FEV₁, FVC, TLC.
   Diffusing Capacity of the Lung for Carbon Monoxide (D_{LCO}): The diffusing capacity of the lungs, measured using carbon monoxide (D_{LCO}) will be conducted according to the ATS/ERS guidelines and will be assessed by the single-breath method. D_{LCO} efforts, up to a maximum of 5, will be performed to produce at least 2 technically acceptable and repeatable traces (according to ATS/ERS guideline criteria). There must be a minimal interval of at least 4 minutes between each effort performed.

4. Safety Assessments

Safety and tolerability will be evaluated throughout this study from signing of the ICF onwards until the EOS visit or date of last contact. The standard safety assessments to evaluate the safety and tolerability of selexipag in this study include reporting and follow-up of SAEs, pregnancies, vital signs, physical examination, ECG, and safety laboratory tests. The study will include the following evaluations of safety and tolerability according to the timepoints provided in Tables 4 and 5.

(i) Physical Examination: Physical examination at Screening and at subsequent visits includes the examination of the general appearance, heart, lungs, extremities, eyes. Other exams will be performed if indicated, based on medical history and/or symptoms. Height will be measured without shoes. Body weight will be measured in indoor clothing but without shoes.

(ii) Vital Signs: Pulse rate, SBP and DBP will be assessed in a supine or sitting position. It is recommended that the participant is allowed to rest for at least 5 minutes before the measurement. It is also recommended that measurements are performed on the same arm and in the same position (supine or sitting) throughout the study for each individual participant. Vital signs are to be measured prior to blood collection.

(iii) Electrocardiogram: A single standard 12-lead ECG will be performed and interpreted locally. During the collection of ECGs, participants should be in a quiet setting without distractions (e.g., television, cell phones). Participants should rest in a supine position for at least 5 minutes before ECG collection and should refrain from talking or moving arms or legs. If blood sampling or vital sign measurement is scheduled for the same timepoint as ECG recording, the procedures should be performed in the following order: ECG(s), vital signs, blood draw.

(iv) Clinical Safety Laboratory Assessments

Blood samples for serum chemistry and hematology will be collected.

5. Adverse Events and Serious Adverse Events

Adverse events will be reported throughout the study from signing of the ICF onwards until the EOS visit or date of last contact.

(i) Time Period and Frequency for Collecting Adverse Event and Serious Adverse Event Information

All Adverse Events: All AEs and special reporting situations, whether serious or non-serious, will be reported throughout the study from signing of the ICF onwards until the EOS visit or date of last contact.

Serious Adverse Events: All SAEs occurring during the study must be reported within 24 hours of their knowledge of the event. Serious adverse events, including those spontaneously reported throughout the study from signing of the ICF onwards until the EOS visit or date of last contact, must be reported, irrespective of the study intervention received by the participant and whether or not this event is considered to be related to study intervention. Serious adverse events occurring after the EOS visit must be reported only if considered to be causally related to previous exposure to the study intervention.

(ii) Pregnancy: All initial reports of pregnancy in female participants must be reported within 24 hours of their knowledge of the event. Pregnancies must be reported as an AE and any AE associated with the pregnancy occurring until the EOS visit or date of last contact must be reported as a separate AE.

(iii) Treatment of Overdose: Overdose is defined by the intake of any single dose greater than 1600 μg or a total daily dose greater than 3200 μg (only in case subjects are on a bid regimen).

(iv) Biomarkers: Biomarker analysis is optional for each participant. Plasma and serum samples for the analysis of pharmacodynamic and disease-related biomarkers can be collected at timepoints indicated in Tables 4 and 5.

H. Statistical Considerations

Data collected during the DB period will be summarized by intervention group and, where appropriate and applicable, compared between intervention groups with hypothesis test. Data collected during the OL period will be summarized overall and by treatment group.

1. Populations for Analyses:

For analysis purposes, the populations are defined in Table 8:

TABLE 8
Overview of the Different Populations for Analyses

| Population | Description |
|---|---|
| Screened Analysis Set (SCR) | All participants who were screened and received a participant |
| | identification number. |
| Full Analysis Set (FAS) | All participants assigned to a study intervention. |
| Hemodynamic Set (HS) | All participants in the hemodynamic cohort. |
| Per-Protocol Analysis Set | All participants who received the assigned study intervention |
| (PPS) | and who complied with the protocol sufficiently to be likely to |
| | exhibit the intervention effects. |
| Safety Analysis Set (SAS) | All participants who received at least one dose of study |
| | intervention. Participants will be analyzed according to the |
| | intervention they actually received. |
| HRCT Analysis Set (HAS) | All participants in FAS who have given consent to participate in |
| | the pulmonary fibrosis substudy. |
| Cardiac MRI Analysis Set | All participants in FAS who have given consent to participate |
| (MAS) | in the cardiac MRI substudy. |
| Selexipag Initiated Set (SIS) | All participants initiated at any time during DB or OL |
| | intervention on selexipag. |

Table 9 defines all analysis sets and their specific usage at all analysis time points. For decision-making at Analysis Timepoints 1 and 2, PVR will be evaluated on the HS. All other efficacy analyses will be primarily evaluated on the FAS. The SIS will be used to evaluate subjects initiated at any time on selexipag. Safety endpoints will be primarily evaluated on the SAS.

TABLE 9
Analysis Sets and Their Usage

| Analysis | | Analysis Set | | | | | | |
|---|---|---|---|---|---|---|---|---|
| Timepoint | Analysis | FAS | HS | PPS | SAS | HAS | MAS | SIS |
| 1 | Interim PVR | | X | | | | | |
| 2 | Final PVR | | X | X | | | | |
| 2 | Interim TTCW | X | | | | | | |
| 2 | Exposure and safety | | | | X | | | |
| 2 | Pulmonary fibrosis | | | | | X | | |
| 2 | MRI | | | | | | X | |
| 2 | Others* | X | | | | | | |
| 3 | Final TTCW | X | | | | | | |
| 3 | Exposure and safety | | | | X | | | |
| 3 | Pulmonary fibrosis | | | | | X | | |
| 3 | MRI | | | | | | X | |
| 3 | Others* | X | | | | | | |
| 4 | Final OL | X | | | X | | | X |

*Demographic and baseline characteristics, medical history, previous and concomitant medications, other efficacy endpoints.

2. Statistical Hypotheses

The hypotheses for the primary endpoint of PVR at peak concentration at Week 20 are formulated in terms of geometric mean (GM) of PVR post to pre percent in participants treated with selexipag (GM_{selexipag}) versus placebo (GM_{placebo}):

H₀: GM_{selixpag}=GM_{placebo}

H_{A}: GM_{selixpag}≠GM_{placebo}

3. Analyses Timepoints

There are 4 Analysis Timepoints:

(a) Analysis Timepoint 1 (futility for PVR)

When approximately 34 participants in the hemodynamic cohort have completed the Week 20 RHC assessment (or discontinued prematurely), an IA for the PVR endpoint will be conducted by an ISSG which is otherwise not involved in the study conduct, analysis or outcome evaluation. The results of the IA will be presented.

(b) Analysis Timepoint 2 (final analysis for PVR and IA for TTCW)

When all participants of the hemodynamic cohort (pre-planned 86 participants) have completed the Week 20 RHC and the Week 26 assessment (or discontinued prematurely), the ISSG will perform the following analyses. First, the final analysis on PVR will be performed.
If PVR is success, i.e., a statistically significant difference between participants receiving selexipag and those receiving placebo, a formal IA on the secondary endpoint TTCW using all data available at this time point will be performed:
   If the observed HR (selexipag/placebo) for TTCW is greater than 1, then the study will be stopped early for futility.
   If the efficacy boundary (Hwang et al., “Group sequential designs using a family of type I error probability spending functions,” Stat. Med. 1990; 9:1439-1445) for TTCW is crossed, the study can be terminated for success. In this case, recruitment will be stopped, and all data will be analyzed by a dedicated submission team which is otherwise not involved in the study conduct or outcome evaluation. The study will continue unchanged for participants already enrolled to allow for the complete assessment of endpoints during the DB period.
   If not terminated early, recruitment will continue until the total sample size of 150 participants has been reached.
If PVR has no success, then the study will be stopped early.

(c) Analysis Timepoint 3 (final analysis for TTCW, end of DB)

When all participants have completed the 52-week DB period (or discontinued prematurely). After the interim database lock for the DB period is done, the study team will be unblinded. The final analysis of the TTCW endpoint will be conducted by the study team. Data for all remaining endpoints will also be analyzed, except analysis for PVR already performed.

(d) Analysis Timepoint 4 (end of OL)

When all participants have completed the OL period (24 months) and safety follow-up (or discontinued prematurely.

4. Sample Size Determination

The study consists of 2 cohorts: the first 86 participants will form the hemodynamic cohort, targeting the primary endpoint of PVR at Week 20. The remaining 64 participants will be enrolled to enrich information on all secondary endpoints. If participant enrollment is not terminated for futility or efficacy at Analysis Timepoint 1 or 2, the total sample size is targeted to be 150 participants.

(i) Sample size for PVR

For PVR, one IA is planned in 34 participants at Analysis Timepoint 1 to allow early termination for futility if the conditional power is less than 10%. The calculation of sample sizes was approximated by Z-test for superiority on the log-transformed PVR percentage of baseline value at Week 20 using the software package EAST™ version 6.4.0.1 for difference of means. A total sample size of 86 participants in the hemodynamic cohort will provide a power of 90% for the treatment effect of a 30% relative decrease (i.e., improvement) in GM as compared to placebo, with a CV of 0.5 and a 2-sided significance level of 0.05. A small amount, i.e., 0.000001, of the significance level will be spent for the futility IA. Assuming a CV of 0.5 for the sample size calculation is a conservative approach and gives an adequate sample size for the IA of TTCW.

The operating characteristics of this design for PVR are summarized in Table 10.

TABLE 10
Operating Characteristics of the Design for PVR

| | | Geometric Mean Ratio | | | |
|---|---|---|---|---|---|
| | | 1 | 0.8 | 0.7 | 0.6 |
| Probability of stopping | CV = 0.4 | 72.7% | 13.9% | 1.7% | 0.06% |
| for futility at IA 1 | CV = 0.5 | 72.5% | 21.8% | 5.5% ^{a} | 0.5% |
| Power | CV = 0.4 | 2.3% | 71.3% | 97.5% | 99.9% |
| | CV = 0.5 | 2.3% | 54.4% | 90.3% ^{a} | 99.4% |

^{a} Data corresponds to the sample size calculation in the paragraph above.

(ii) Sample size for TTCW

Data for TTCW according to the GIMP definition have not been reported in SAPH. The SSc subtype of PAH is considered a proxy for SAPH. For this rare disease the expected accrual rate is approximately 5 participants per month. Each participant will be followed up until Week 52 under the DB period. To reach 90% power with 2-sided significance level 0.05, the required sample sizes and durations of the DB period under scenarios A and B, without considering IAs, are calculated using EAST version 6.4.0.1 and presented in Table 11.

TABLE 11
Required Sample Sizes and Durations of the Double-Blind
Period for 90% Power and 0.05 2-Sided Significance Level
Under 2 Different Scenarios Without Interim Analyses

| | Scenario | Scenario |
|---|---|---|
| | A | B |
| Median TTCW in months (selexipag vs placebo) | 18 vs 12 | 15 vs 11.5 |
| Hazard ratio | 0.667 | 0.762 |
| Total sample size | 588 | 1210 |
| Expected number of events at end of DB period | 256 | 569 |
| Threshold for observed hazard ratio to declare | 0.783 | 0.848 |
| significance | | |
| Duration of double-blind period in months | 130 | 254 |

A sample size of 150 participants (i.e., 86 participants in the hemodynamic cohort and 64 participants in the non-hemodynamic cohort) for TTCW was selected based on feasibility considerations. Assuming an accrual rate of 5 participants per month, it will take approximately 42 months to complete the DB period.

An IA for TTCW is planned at Analysis Timepoint 2 using all available data, i.e., including also participants who have not completed Week 26 assessments. If the observed hazard ratio is greater than 1, then the study will be stopped early for futility. In order not to miss the potential efficacy signal in TTCW with the constrained sample size, a large 2-sided significance level of 0.3 is chosen. The main statistical inference will be based on 2-sided 70% CI for hazard ratio.

The expected number of events, power and probabilities of early stop under scenarios A and B are summarized in Table 12.

TABLE 12
Expected Number of Events, Power and Probabilities of Early
Stop Under 2 Different Scenarios (A and B) for a Sample Size
of 150 Participants and a 0.3 2-Sided Significance Level

| | Scenario | Scenario |
|---|---|---|
| | A | B |
| Median TTCW in months (Selexipag vs placebo) | 18 vs 12 | 15 vs 11.5 |
| Hazard ratio | 0.667 | 0.762 |
| Expected number of events at end of the DB | 66 | 71 |
| period | | |
| Probability of stopping for futility at IA | 10.3% | 18.9% |
| Probability of stopping for efficacy at IA | 9.9% | 4.8% |
| Overall power (at the end of the DB period) | 71.4% | 52.7% |

The amount of significance level to be spent at each IA will be determined by the Hwang, Shih and DeCani's error spending function with parameter γ=−8:

$$α ( t ) = α ( t , γ ) = α \frac{1 - {e}^{- γ t}}{1 - e {e}^{- γ}}$$

The exact value of significance level α(t) will depend on the actual information fraction t, which is the actual number of observed events divided by the required total number of events under Scenario B in Table 12. If there is no treatment-effect on TTCW, then the probability of stopping for futility at IA 2 is around 50%.

5. Statistical Analyses

(i) Primary Efficacy Analysis

Definition of PVR endpoint: The primary endpoint is PVR at peak concentration at Week 20.

Estimand

The primary PVR estimand is described according to the following four attributes:

Population: HS (all randomized subjects), as randomized.

Variable: Absolute change from baseline to Week 20 visit of the natural log-transformed PVR values. The results will be presented on the original scale (geometric means, GM) after exponentiation of the absolute mean changes obtained on the natural log scale:

The ratio of the PVR value post-intervention initiation at Week 20 (post) vs the PVR value pre-intervention initiation at baseline (pre), expressed as a percentage, i.e.:

$$PVR post to pre percent = ( \frac{PVR at Week 20}{PVR at beaseline} ) ⨯ 100 ( \% )$$

Intercurrent events (events that preclude observation of the variable or affect its interpretation): Missing PVR assessments at baseline or at Week 20 will be imputed specific to the reason for missing data.

Population-level summary: Ratio of the geometric means of the PVR post to pre percent between selexipag and placebo.

As a summary measure, the difference in means of natural logarithm transformed PVR post to pre percent between selexipag and placebo, or equivalently ratio of GM of PVR post to pre percent between selexipag and placebo will be calculated, i.e.:

$$\frac{GM ( PVR post to pre percent in selexipag )}{GM ( PVR post to pre percent in placebo )}$$

This estimand targets the effect of intervention initiation on the variable measurement and follows an “intent to treat” strategy.

Hypotheses: The hypotheses for the primary endpoint of PVR at peak concentration at Week 20 are formulated in terms of GM of PVR post to pre percent (as defined above) in participants treated with selexipag (GM_{selexipag}) versus placebo (GM_{placebo}):

H₀: GM_{selexipag}=GM_{placebo}

H_{A}: GM_{selexipag}≠GM_{placebo}

The null hypothesis is planned to be tested in the HS by means of an ANCOVA model on the natural logarithm transformed PVR post to pre percent. Model covariates will include randomized intervention, stratification factor and the natural logarithm transformed baseline PVR value.

Handling of missing data: Imputation methods for PVR will be specific to the reason for missing data. The baseline reference value for PVR is based on the last RHC performed prior to DB study intervention initiation. If PVR cannot be calculated due to missing PAWP, the following conventions will be applied for the calculation at a visit at which both mPAP and CO are assessed:

1. If only PAWP is missing, then the LVEDP will be used.

2. If PAWP and LVEDP are missing both at baseline and post-baseline, the missing PAWP is imputed using the median of all values observed at the respective time point in all subjects from the same randomized intervention group.

3. If PAWP and LVEDP are missing either at baseline or at post-baseline, the available PAWP for the subject is used as a substitute for the missing PAWP.

In the case of a missing PVR value at Week 20, the last available post-baseline value obtained before the Week 20 analysis time window is carried forward. All observed PVR values will be used to derive the imputed values.

This imputation will be performed except in the following cases:
If a participant dies without a Week 20 value, then the missing PVR post to pre percent value is imputed with the largest PVR post to pre percent value at Week 20 amongst all participants in the same intervention group and analysis set. The resulting imputed Week 20 PVR is the product of this imputed PVR post to pre percent value and the respective baseline PVR value.
If the participant is alive and does not have a post-baseline value, then the missing PVR post to pre percent value is imputed with the 50^{th} percentile of the PVR post to pre percent values from all participants in the same intervention group and analysis set. The resulting imputed Week 20 PVR is the product of this imputed PVR post to pre percent value and the respective baseline PVR value.

Main analysis: The interim and final analyses of the primary efficacy endpoint with observed or imputed values will be tested in the HS using the ANCOVA model. From the ANCOVA model, PVR post to pre percent will be summarized by intervention group using covariate-adjusted GM and corresponding 2-sided 95% CI. The between-group ratio of GM with corresponding unadjusted 95% 2-sided CI and p-value will be displayed as the placebo-corrected intervention effect. To adjust for multiple testing, repeated CIs for intervention effect at Analysis Time Points 1 and 2 will be presented. For each intervention group, the least square mean and 2-sided 95% CI of the natural logarithm of the PVR post to pre percent will be inversely transformed using the exponential function and multiplied by 100 to provide the GM of the PVR post to pre percent and the corresponding 2-sided 95% CI, expressed as a percentage. Absolute values at baseline and at Week 20 as well as absolute pre-post changes from baseline to Week 20 in PVR will be summarized using descriptive statistics.

Supportive/sensitivity analyses: Sensitivity analyses will be conducted using alternative imputation rules to assess the imputation assumptions of the main statistical analysis for PVR at Week 20. Furthermore, a sensitivity analysis of PVR at Week 20 will be conducted in the per-protocol analysis set to assess the robustness of the results of the main statistical analysis against protocol deviations leading to exclusion from the PPS.

(ii) Secondary Efficacy Analyses

Time to clinical worsening (according to the CHMP definition) up to Week 52.

Endpoint definition: This endpoint will be derived from relevant data (all-cause death, non-planned PH-related hospitalization, WHO FC, signs or symptoms of RHF, and 6MWD). The time to clinical worsening will be calculated from date of randomization to the date of onset of the first component event. For participants who have not experienced an event by the respective analysis cut-off date, their TTCW will be right censored at the latest available visit/contact date in the DB period up to the analysis cut-off date.

Main analysis: A 2-sided stratified (for the stratification factors) log-rank test will be applied. Kaplan-Meier estimates for the survival functions will be provided together with 2-sided 95% CI. The hazard ratio will be estimated using a Cox model, including intervention group, stratification factor and participation in the hemodynamic cohort as covariates. The frequency of each component event will also be presented. The sample size cannot provide sufficient power for TTCW (see Table 12). The intervention effects in TTCW will be summarized by the corresponding 2-sided 70% CI for hazard ratio at Analysis Timepoints 2 and 3.

Longitudinal trend of 6MWD up to Week 52.

Endpoint definition: All 6MWD obtained at different time points up to Week 52 for each participant will be included in the analysis.

Main analysis: The data will be analyzed using a mixed model, including covariates for intervention group, stratification factor and participation in the hemodynamic cohort. The model will include random effects for baseline, intercept and slope across patients. The effect of intervention (selexipag vs placebo) will be assessed on baseline vs post-baseline, on the slope, and on the estimated change from baseline to Week 52.

Proportion of participants with oxygen desaturation (SpO₂) post 6MWT up to Week 52.

Endpoint definition: Oxygen desaturation (SpO₂) is defined as a decrease in post-6MWT oxygen saturation by at least 5% from the respective pre-6MWT oxygen saturation value.

Main analysis: The data will be analyzed by a Cochran-Mantel-Haenszel test adjusted by intervention, stratification factor and participation in the hemodynamic cohort. The common odds ratio and CI will be estimated, provided there is no clear evidence against a common odds ratio across the strata (evaluated by the Breslow-Day test).

Proportion of participants with improvement/no change/worsening versus baseline in WHO FC at Week 52.

Endpoint definition: Improvement/no change/worsening from Baseline to Week 52 in WHO FC is defined as decrease/no change/increase in FC as compared to WHO FC at baseline.

Main analysis: The data will be analyzed by a Cochran-Mantel-Haenszel test adjusted by WHO FC at baseline, intervention, stratification factor and participation in the hemodynamic cohort.

Proportion of participants with all-cause death or PH-related hospitalizations up to Week 52.

Endpoint definition: This is the proportion of participants who have experienced at least 1 PH-related hospitalization or died up to Week 52, within each intervention group.

Main analysis: A logistic regression model including intervention group, stratification factor and participation in the hemodynamic cohort as covariates will be used to analyze the data.

Rate of all-cause death or PH-related hospitalizations up to Week 52.

Endpoint definition: This rate will be expressed as per 100-participant years, calculated by dividing the total number of all-cause death or hospitalizations related to PH-worsening by the cumulative exposure up to Week 52 of all participants in each intervention group.

Main analysis: A negative-binomial regression model including intervention group, stratification factor and participation in the hemodynamic cohort as covariates and with over-dispersion will be used to analyze the data. The different durations of follow-up time across participants will be considered in the offset variable.

Change from baseline up to Week 52 in SF-12 scores.

Endpoint definition: The physical and mental component summary scores will be derived according to the instrument's instruction. For each summary score, the change from baseline to Week 52 will be calculated for each participant.

Main analysis: The change from baseline to Week 52 will be analyzed by means of an ANCOVA model and will include intervention, baseline score value, stratification factors and participation in the hemodynamic cohort as covariates. Least squares estimates for each intervention group and for the placebo-corrected intervention effect will be displayed with means, 95% CIs and p-values.

Change from baseline up to Week 52 in the KSQ scores.

Endpoint definition: An overall summary score will be derived according to the instrument's instruction. The change from baseline to Week 52 will be calculated for each participant.

Main analysis: The change from baseline to Week 52 in the overall KSQ score will be analyzed by means of an ANCOVA model and will include intervention, baseline score value, stratification factor and participation in the hemodynamic cohort as covariates. Least squares estimates for each intervention group and for the placebo-corrected intervention effect will be displayed with means, 95% CIs and p-values.

Change from baseline up to Week 39 in the PAH-SYMPACT scores.

Endpoint definition: The symptoms and impact summary scores will be derived according to the instrument's instruction. For each summary score, the change from baseline to Week 39 will be calculated for each participant.

Main analysis: The change from baseline to Week 39 in each SYMPACT™ score will be analyzed by means of an ANCOVA model and will include intervention, baseline score value, stratification factor and participation in the hemodynamic cohort as covariates. Least squares estimates for each intervention group and for the placebo-corrected intervention effect will be displayed with means, 95% CIs and p-values. Responder analyses will also be implemented on PAH-SYMPACT™ scores and will be defined in the SAP.

Change from baseline up to Week 52 in the PGA-S scores.

Endpoint definition: The change from baseline to Week 52 will be calculated for each participant. Hypotheses: The null hypothesis is that there is no difference between selexipag and placebo in the mean change from baseline to Week 52. The alternative hypothesis is that there are some differences between intervention groups.

Main analysis: The change from baseline to Week 52 will be analyzed by means of an ANCOVA model and will include intervention, baseline score value, stratification factor and participation in the hemodynamic cohort as covariates. Least squares estimates for each intervention group and for the placebo-corrected intervention effect will be displayed with means, 95% CIs and p-values.

Change from baseline up to Week 52 in the CGI-S scores: Endpoint definition, hypotheses and main analysis are as for change from baseline up to Week 52 in the PGA-S scores.

CGI-C scores up to Week 52: Endpoint definition, hypotheses and main analysis are as for change from baseline up to Week 52 in the PGA-S scores.

(iii) Safety Analyses

Adverse Events: The verbatim terms to identify AEs will be coded using the MedDRA. Intervention-emergent adverse events are AEs with onset during the intervention period or that are a consequence of a pre-existing condition that has worsened since baseline. All reported AEs will be included in the analysis. For each AE, the percentage of participants who experience at least 1 occurrence of the given event will be summarized by intervention group. In addition, comparisons between intervention groups will be provided if appropriate.

Clinical Laboratory Tests: Descriptive statistics will be calculated for each laboratory analyte at baseline and for observed values and changes from baseline at each scheduled timepoint by intervention group. Frequency tabulations of the abnormalities will be made.

Vital Signs: Descriptive statistics of pulse rate, SBP and DBP values and changes from baseline will be summarized at each scheduled timepoint by intervention group.

Supplemental Oxygen Rate: Descriptive statistics of values and changes from baseline in supplemental oxygen rate will be summarized by intervention group.

(iv) Biomarkers Analyses: Biomarker samples will be used to generate plasma and serum marker data for computational analyses. Changes in plasma and serum biomarkers over time will be summarized by intervention group. Associations between baseline levels and changes from baseline in select markers and clinical response will be explored.

### Example 3

The overall benefit/risk assessment for this clinical study is considered acceptable because there is an unmet medical need for therapeutic options for patients with SAPH.

Safety will be closely monitored throughout the study:
In general, safety evaluations (including but not limited to thyroid function tests, blood pressure monitoring, hematology laboratory tests) will be performed at scheduled visits during the study, as indicated in Tables 14 and 15.
Any clinically significant abnormalities (including those persisting at the end of the study/early withdrawal) will be followed until resolution or until a clinically stable condition is reached.

Participants will discontinue study intervention for the reasons discussed herein.

A. Objectives and Endpoints

Table 13 provides the objectives and endpoints of the methods described herein:

TABLE 13

| | |
|---|---|
| Objectives | Endpoints |

Primary

| | |
|---|---|
| To assess the effect of selexipag | PVR on study intervention up to Week 26 |
| versus placebo on pulmonary | expressed as percent of the baseline value. |
| vascular resistance (PVR) in | |
| participants with | |
| sarcoidosis-associated pulmonary | |
| hypertension (SAPH) up to Week | |
| 26. | |

Exploratory

| | |
|---|---|
| To evaluate the effect of selexipag | TTCW up to EOMOP defined as at least one of |
| versus placebo on time to clinical | the following components: |
| worsening (TTCW). | All-cause death |
| | Unplanned PH-related hospitalization |
| | Increase in WHO functional class (FC) |
| | Lung transplantation |
| | Atrial balloon septostomy |
| | Initiation of parenteral or new class of |
| | PH-specific therapy for clinical |
| | worsening |
| To evaluate the effect of selexipag | Change from baseline in 6MWD, Borg |
| versus placebo on exercise | Dyspnea Index (BDI), oxygen saturation, and |
| capacity. | WHO FC at Week 39 and over time. |
| | Proportion of participants with oxygen |
| | desaturation post 6-minute walk test (6MWT) |
| | at Week 39 and over time (identified by |
| | decrease in oxygen saturation [SpO₂] by at |
| | least 5% from pre-6MWT). |
| To evaluate the effect of selexipag | Change from baseline to Week 39 in |
| vs placebo on daily life physical activity | actigraphy-assessed DLPA as measured by: |
| (DLPA) and sleep parameters | Total DLPA in counts per minute |
| | Total volume of activity (above sedentary) |
| | Daily time spent (minutes) in non- |
| | sedentary activity |
| | Percentage of daily time spent in non- |
| | sedentary activity |
| | Moderate to vigorous physical activity |
| | (MVPA) |
| | Time spent in the different activity |
| | categories |
| | Change from baseline to Week 39 in sleep |
| | parameters: |
| | Total sleep time (TST; minutes) |
| | Wake after sleep onset (WASO; minutes) |
| | Number of awakenings |
| | Sleep efficiency (percentage) |
| To evaluate the effect of selexipag | Proportion of participants with improvement, |
| versus placebo on WHO FC. | worsening and no change from baseline in |
| | WHO FC at Week 39 and over time. |
| To evaluate the effect of selexipag | Rate of all-cause death or unplanned PH- |
| versus placebo on death or PH- | related hospitalization up to EOMOP. |
| related hospitalizations | Time to all-cause death up to EOMOP. |
| To evaluate the effect of selexipag | Change from baseline up to Week 39 in SF-12 |
| versus placebo on patient-reported | scores. |
| outcomes (PROs) assessed by the | Change from baseline up to Week 39 in KSQ |
| 12-Item Short Form Health Survey | scores. |
| (SF-12), King's sarcoidosis | Change from baseline up to Week 39 in |
| questionnaire (KSQ), Pulmonary | PAH-SYMPACT scores. |
| Arterial Hypertension-Symptoms | Change from baseline up to Week 39 in PGA- |
| and Impact (PAH-SYMPACT), and | S scores. |
| Patient Global Assessment of | |
| Severity (PGA-S) | |
| To evaluate the effect of selexipag | Change from baseline up to Week 39 in CGI-S |
| versus placebo on clinician- | scores. |
| reported outcomes (CROs) | Change from baseline up to Week 39 as |
| assessed by the Clinician Global | measured by CGI-C scores. |
| Impression of Severity (CGI-S) | |
| and Clinician Global Impression of | |
| Change (CGI-C) | |
| To evaluate the effect of selexipag | Absolute and change from baseline in the |
| on the number of PH low-risk | number of low-risk criteria based on WHO FC, |
| criteria | 6MWD, N-terminal pro b-type natriuretic |
| | peptide (NT-proBNP), and Cardiac Index (CI) |
| | up to Week 26. |
| To evaluate the effect of selexipag | Change from baseline in NT-proBNP serum |
| on NT-proBNP | levels up to Week 39. |
| To evaluate the effect of selexipag | Change from baseline in serum biomarkers and |
| on disease and pathway-related | associations between serum biomarker levels |
| serum biomarkers | and clinical response and baseline |
| | characteristics up to Week 39. |
| To evaluate the change in | Change from baseline in other hemodynamic |
| hemodynamic variables other than | variables (including cardiac output [CO], CI, |
| PVR | mean right atrial pressure [mRAP], mean |
| | pulmonary arterial pressure [mPAP], mixed |
| | venous oxygen saturation [SVO₂]) up to Week |
| | 26. |

Safety

| | |
|---|---|
| To assess the overall safety of | Intervention-emergent AEs. |
| selexipag | Intervention-emergent prostacyclin-associated |
| | AEs. |
| | Serious adverse events (SAEs) up to End-of- |
| | Study (EOS). |
| | AEs leading to premature discontinuation of |
| | study intervention. |
| | Intervention-emergent AEs of special interest |
| | (e.g., hypotension, anemia, hyperthyroidism). |
| | Change in vital signs (systolic and diastolic |
| | arterial blood pressure and pulse rate) and |
| | body weight from baseline to all assessed |
| | timepoints during the study. |
| | Intervention-emergent marked laboratory |
| | abnormalities. |
| | Change from baseline in forced vital capacity |
| | (FVC) and diffusing capacity of the lung for |
| | carbon monoxide (D_{LCO}). |
| | Change from baseline in supplemental oxygen |
| | rate. |
| | Change from baseline in arterial blood gas |
| | parameters. |

B. Study Design

1. Overall Design

This is a prospective, randomized, double-blind (DB), placebo-controlled, multicenter, interventional study in seventy-four subjects, i.e., men and women ≥18 and ≤75 years of age with SAPH. Sixty four of the subjects were adults (18-64 years of age), and fourteen were elderly (≥65 years of age). Study intervention will be up-titrated to allow each participant to reach their individual maximum tolerated dose (iMTD), in the range of 200 μg to 1600 μg twice daily (bid). For participants with moderate hepatic impairment (Child-Pugh B) or who are concomitantly taking (a) moderate cytochrome P450 (CYP)2C8 inhibitor(s) the dosing frequency is qd. Diagrams of the study design and titration are provided in FIGS. 14 and 15, respectively.

The study starts with the first ICF signed by the first participant and ends with the last safety follow-up TC or visit of the last participant. The study comprises the following periods:

(a) A screening period of up to 30 days: starts with the signature of the ICF and ends with the participant's randomization at Visit 2, Day 1. The screening period should last at least 14 days to allow collection of baseline data for daily life physical activity (DLPA), sleep parameters and PAH-SYMPACT™.

(b) Intervention and observation periods:
A main observation period (MOP) which starts with the participants randomization at Visit 2, Day 1 and with a titration phase of up to 12 weeks. Participants receive double-blind study intervention (selexipag or placebo) during this period. It ends on the day of the EOMOP visit. The EOMOP visit is the data cut-off for the primary efficacy and safety analyses. The EOMOP visit for all participants is planned 39 Weeks+/−1 month after randomization of the last participant. The duration of the MOP will be different for each individual participant and will depend on the time of each participant's individual date of randomization.

Participants who prematurely discontinue study intervention before the EOMOP visit will continue to perform visits and assessments as scheduled until the EOMOP visit.

For participants who prematurely discontinue study intervention before the EOMOP visit and who disagree to perform visits and assessments as scheduled until the EOMOP visit long-term follow-up information regarding their survival status will be collected yearly until death or the time of EOMOP.

Information on the required procedures to follow if participants discontinue the study intervention prematurely is provided herein.
A DB extension period extending intervention for participants who do not prematurely discontinue study intervention before EOMOP. The period starts in the evening of the day of the EOMOP visit and ends with the EOT visit. This period will last approximately 5 months. All participants entering the DB extension period will continue taking study intervention (selexipag or placebo) during this period. Study intervention allocation will be unblinded approximately 1 month before the EOT visit.

(c) A safety follow-up period which starts on the day after the last dose of study intervention and ends 30 (+5) days later with the safety follow-up TC. Diagrams of the study design and titration are provided in FIGS. 14 and 15, respectively.

Approximately 74 participants will be recruited.

The visit schedule and protocol-mandated procedures are performed according to the Schedule of Activities Table 14 and Table 15.

TABLE 14
Schedule of Activities - while taking double-blind study intervention

| | | Screen- | Main Observation Period on-treatment | | | | | | |
|---|---|---|---|---|---|---|---|---|---|
| Periods | Name | ing | (on double-blind study intervention) | | | | | | |
| | | | | | | | | | TC |
| | | | | | | | | | & every |
| | | | | Weekly | | | | | 6 |
| Visits | Number | 1 | 2 | TC | 3 | 4 | 5 | 6 | months |
| | | | | Titration | | | | | |
| | | | | Phase | | | | | Week |
| | | | | (W 1, | | | | | 52, |
| | | | | W 2, | | | | | 78, |
| | | Screen- | Random- | W 3, | Week | Week | Week | Week | 104, |
| | Name | ing | ization | etc.) | 12 | 20 | 26 | 39 | etc |
| | | | | Day 8 | | | | | |
| | | | | (±3 | | | | | |
| | | | | days) and | | | | | |
| | | | | weekly | | | | | Day |
| | | | | (±3 | | | | | 365, |
| | | | | days) | | | | | 547, |
| | | | | until | Day | Day | Day | Day | 729, |
| | | Day −30 | | Day 78 | 85 | 141 | 183 | 274 | etc. |
| | | to | | (±3 | (±7 | (±7 | (±7 | (±7 | (±7 |
| | Time | Day −1 | Day 1 | days) | days) | days) | days) | days) | days) |

Screening/Administrative

| | | |
|---|---|---|
| ICF ⁰ | X | |
| Optional | X | |
| biomarker ICF | | |
| Demographics and | X | |
| height | | |
| Review medical | X | |
| history | | |
| requirements | | |
| Inclusion/exclusion | X | X ⁰ |
| criteria | | |
| Prestudy therapy | X | X |
| Serum pregnancy | X | |
| test (WOCBP) ^{0; 0} | | |

| | | |
|---|---|---|
| Urine pregnancy | X | monthly (±7 days) |
| test (WOCBP) ⁰ | | |

Study Intervention Administration

| | | | | | | | |
|---|---|---|---|---|---|---|---|
| Randomization | X | | | | | | |
| Study intervention | X | | X | X | X | X | X |
| dispensing/return | | | | | | | |
| Study intervention | X | X | X | X | X | X | X |
| administration ⁰ | | | | | | | |

Efficacy Evaluations

| | | | | | | |
|---|---|---|---|---|---|---|
| RHC | X ⁰ | | | | X ⁰ | |
| 6MWT/BDI/ | X | X | X ⁰ | X ⁰ | X ⁰ | X ⁰ |
| SpO₂ | | | | | | |
| WHO FC | X | X | X | X | X | X |
| Actigraphy ⁰ | X ⁰ | | X | X | X | X |
| SF-12 ^{0,} King's | X | | X | X | X | X |
| SQ ⁰, PGA-S ⁰ | | | | | | |
| PAH- | X ⁰ | | X | X | X | X |
| SYMPACT ™ ⁰ | | | | | | |
| CGI-S ⁰ | X | | X | X | X | X |
| CGI-C ⁰ | | | X | X | X | X |
| NT-proBNP ⁰ | | X | X | X | X | X |

Safety Evaluations

| | | | | | | |
|---|---|---|---|---|---|---|
| Vital signs (BP, | X | X | X | X | X | X |
| HR), weight | | | | | | |
| Local 12-lead | X | | | | | X |
| ECG (single) | | | | | | |
| Physical | X | | | | X | |
| examination ⁰ | | | | | | |
| Arterial Blood | X ⁰ | | | | X ⁰ | |
| Gas (ABG) | | | | | | |
| DLCO | X | | | | X | |
| Spirometry | X | | | | X | |

Clinical Laboratory Tests

| | | | | | | |
|---|---|---|---|---|---|---|
| Hematology and | X | X | X | X | X | X |
| clinical chemistry ⁰ | | | | | | |

Biomarkers

| | | | | |
|---|---|---|---|---|
| Seram and plasma | X | X | X | X |
| samples for | | | | |
| exploratory | | | | |
| biomarker | | | | |
| research ^{0; 0} | | | | |

Ongoing Participant Review

| | | | | | | | | |
|---|---|---|---|---|---|---|---|---|
| Concomitant | | | X | X | X | X | X | X |
| Therapy | | | | | | | | |
| SAEs/AEs ⁰ | X | X | X | X | X | X | X | X |

| | | Main Observation | | | | |
|---|---|---|---|---|---|---|
| | | Period on-treatment | | DB | Safety | |
| | | (on double-blind | | Extension | Follow- | |
| Periods | Name | study intervention) | | Period | Up | |
| | | | | | Safety | |
| | | Visit | | | follow- | |
| | | & every | | | up | |
| | | 6 | | | phone | U1, 2, |
| Visits | Number | months | EOMOP | EOT | call | 3, etc. |
| | | Week | | | Safety | |
| | | 65, | | | follow- | |
| | | 91, | | | up | Unsched- |
| | | 117, | | | phone | uled |
| | Name | etc. | EOMOP ⁰ | EOT | call | visit ⁰ |
| | | | Announced | | | |
| | | | (within ±1 | | | |
| | | Day | months | | | |
| | | 456, | of the | Announced | | |
| | | 638, | planned | (approxi- | 30 | |
| | | 819, | Visit 6 | mately | (±5) | |
| | | etc. | for the | 5 months | days | |
| | | (±7 | last | after | after | Any |
| | Time | days) | participant) | EOMOP) | EOT | time |

Screening/Administrative

ICF ⁰
Optional
biomarker ICF
Demographics and
height
Review medical
history
requirements
Inclusion/exclusion
criteria
Prestudy therapy
Serum pregnancy
test (WOCBP) ^{0; 0}

| | | |
|---|---|---|
| Urine pregnancy | monthly (±7 days) | X |
| test (WOCBP) ⁰ | | |

Study Intervention Administration

| | | | |
|---|---|---|---|
| Randomization | | | |
| Study intervention | X | X | X |
| dispensing/return | | | |
| Study intervention | X | X | |
| administration ⁰ | | | |

Efficacy Evaluations

| | | | | |
|---|---|---|---|---|
| RHC | | | | |
| 6MWT/BDI/ | X ⁰ | X ⁰ | X ⁰ | X ^{0; 0} |
| SpO₂ | | | | |
| WHO FC | X | X | X | X ⁰ |
| Actigraphy ⁰ | | | | |
| SF-12 ^{0,} King's | | | | |
| SQ ⁰, PGA-S ⁰ | | | | |
| PAH- | | | | |
| SYMPACT ™ ⁰ | | | | |
| CGI-S ⁰ | | | | |
| CGI-C ⁰ | | | | |
| NT-proBNP ⁰ | | | | |

Safety Evaluations

| | | | | |
|---|---|---|---|---|
| Vital signs (BP, | X | X | X | X ⁰ |
| HR), weight | | | | |
| Local 12-lead | | | X | |
| ECG (single) | | | | |
| Physical | X | | X | X ⁰ |
| examination ⁰ | | | | |
| Arterial Blood | | | | |
| Gas (ABG) | | | | |
| DL_{CO} | | | X | |
| Spirometry | | | X | |

Clinical Laboratory Tests

| | | | |
|---|---|---|---|
| Hematology and | X | X | X |
| clinical chemistry ⁰ | | | |

Biomarkers

Seram and plasma
samples for
exploratory
biomarker
research ^{0; 0}

Ongoing Participant Review

| | | | | | |
|---|---|---|---|---|---|
| Concomitant | X | X | X | | X ⁰ |
| Therapy | | | | | |
| SAEs/AEs ⁰ | X | X | X | X | X ⁰ |

a. The EOMOP visit will have to be performed within ±1 month of the planned Visit 6/Week 39 of the last participant and will be announced approximately 9 months in advance. For the last participant the EOMOP visit will be Visit 6/Week 39. For all other participants, if the EOMOP visit falls within the visit window of any other scheduled visit, these visits can be combined, and assessments will not be repeated.
b. Unscheduled visits may be performed at any time during the study. Assessments (other than reporting of AE/SAE and concomitant medication) are to be performed at the discretion of the investigator and are reported in the eCRF.
Footnote 0 indicates which assessments are mandatory.
c. In case an unscheduled visit is performed this assessment must be performed and recorded in the eCRF.
d. Must be signed before first study-related activity.
e. A serum pregnancy test must be performed at Screening or in case of a positive urine pregnancy test.
f. Transferred electronically by an external service provider.
g. Assessment not collected in eCRF.
h. In case of bid dosing, first dose to be taken in the evening of Day 1. In case of qd dosing, the first dose of study intervention should be taken in the morning of Day 2. Includes study intervention up-titration.
i. A historical RHC is allowed. If no historical results are available, an RHC must be performed during the Screening period only if all other inclusion criteria are met and none of the exclusion criteria is met.
j. If the 6MWT and RHC are assessed on the same day the RHC should be performed after the 6MWT and within 2 to 5 hours post-dose.
k. To be performed within 2 to 5 hours post-dose and prior to the RHC (if applicable).
l. To collect baseline data, participants should wear the actigraphy device 14 days between Visit 1 (Screening) and Visit 2 (Randomization). It is recommended to start on the day following the screening visit. If required, the baseline RHC should not to be done during this time.
m. To assess baseline, the PAH-SYMPACT questionnaire should be completed on 7 consecutive days starting on the day following the screening visit. If required, the baseline RHC should not to be done during the 7 consecutive days of the PAH-SYMPACT completion.
n. If the results for the screening blood samples from the central laboratory are not available in time for randomization of the participant, an additional blood sample may be drawn to verify eligibility based on a local laboratory test.
o. A historical ABG is allowed. If no historical results are available, an ABG must be performed during the Screening period.
p. The ABG can be done during the RHC.
q. All AE and SAEs will be reported throughout the study from signing of the ICF onwards until the EOS visit or death or lost to follow-up.
r. Optional assessment.

TABLE 15
Schedule of Activities - after premature discontinuation of study intervention before EOMOP

| | | | | Main Observation Period Off-treatment | | | | | | | | Survival |
|---|---|---|---|---|---|---|---|---|---|---|---|---|
| | | | | (after premature discontinuation of double-blind | | | | | | | | Follow- |
| Periods | Name | Safety Follow-Up | | study intervention) | | | | | | | | Up |
| | | | Safety | | | | | | | | | |
| | | | follow- | | | | | | | | | |
| | | | up | | | | | TC & | Visit & | | | |
| | | Premature | phone | | | | | every 6 | every 6 | | u1, 2, 3, | SFU 1, 2, 3, |
| Visits | Number | EOT | call | 3 | 4 | 5 | 6 | months | months | EOMOP | etc. | etc. |
| | | | Safety | | | | | Week | Week | | | |
| | | | follow- | | | | | 52, | 65, | | | |
| | | | up | | | | | 78, | 91, | | Unsched- | Survival |
| | | Premature | phone | Week | Week | Week | Week | 104, | 117, | | uled | follow- |
| | Name | EOT ⁰ | call | 12 | 20 | 26 | 39 | etc. | etc. | EOMOP ⁰ | visit ⁰ | up ⁰ |
| | | | | | | | | | | | | Yearly after |
| | | | 30 | | | | | | | | | study |
| | | Within | (±5) | | | | | | | | | discontin- |
| | | 7 days | days | | | | | Day | Day | Announced | | uation until |
| | | after | after | | | | | 365, | 456, | (within ±1 | | death or ±1 |
| | | last | last | | | | | 547, | 638, | month of the | | month of the |
| | | study | study | Day | Day | Day | Day | 729, | 819, | planned | | planned |
| | | inter- | inter- | 85 | 141 | 183 | 274 | etc. | etc. | Visit 6 for | | Visit 6 for |
| | | vention | vention | (±7 | (±7 | (±7 | (±7 | (±7 | (±7 | the last | | the last |
| | Time | dose | dose | days) | days) | days) | days) | days) | days) | participant) | Any time | participant |

Screening/Administrative

| | | |
|---|---|---|
| Serum pregnancy | | |
| test (WOCBP) ^{0, 0} | | |
| Urine pregnancy | X | X |
| test (WOCBP) ⁰ | | |

Study Intervention Administration

| | |
|---|---|
| Study intervention | X |
| dispensing/return | |

Efficacy Evaluations

| | | | | | | | | |
|---|---|---|---|---|---|---|---|---|
| RHC | X ^{0, 0} | | | | | | | |
| 6MWT/BDI/SpO₂ | X | X | X | X | X | X | X | X ⁰ |
| WHO FC | X | X | X | X | X | X | X | X ⁰ |
| Actigraphy ^{0; 0} | X | X | X | X | X | | | |
| SF-12 ^{0; 0}, King's | X | X | X | X | X | | | |
| SQ ^{0; 0}, | | | | | | | | |
| PGA-S ^{0; 0} | | | | | | | | |
| PAH-SYMPACT ™ ^{0; 0} | X | X | X | X | X | | | |
| CGI-S ^{0; 0} | X | X | X | X | X | | | |
| CGI-C ^{0; 0} | X | X | X | X | X | | | |
| NT-proBNP ^{0; 0} | X | X | X | X | X | | | |

Safety Evaluations

| | | | | | | | | |
|---|---|---|---|---|---|---|---|---|
| Vital signs (BP, | X | X | X | X | X | X | X | X ⁰ |
| HR), weight | | | | | | | | |
| Local 12-lead ECG | X | | | | X | | | |
| (single) | | | | | | | | |
| Physical | X | | | X | | X | | X ⁰ |
| examination ⁰ | | | | | | | | |
| Arterial Blood Gas | X ⁰ | | | | | | | |
| (ABG) | | | | | | | | |
| DLCO | X | | | X | | | | |
| Spirometry | X | | | X | | | | |

Clinical Laboratory Tests

| | | | | | | | |
|---|---|---|---|---|---|---|---|
| Hematology and | X | X | X | X | X | X | X |
| clinical chemistry ^{g} | | | | | | | |

Biomarkers

| | |
|---|---|
| Serum and plasma | X |
| samples for | |
| exploratory | |
| biomarker research ^{0, 0; 0} | |

Ongoing Participant Review

| | | | | | | | | | | | |
|---|---|---|---|---|---|---|---|---|---|---|---|
| Concomitant | X | | X | X | X | X | X | X | X | X ⁰ | |
| Therapy | | | | | | | | | | | |
| SAEs/AEs ⁰ | X | X | X | X | X | X | X | X | X | X ⁰ | |
| Survival ⁰ | | | | | | | | | | | X |

a. The premature EOT visit is the first visit after premature discontinuation of study intervention. For participants who continue with visits and assessments if the premature EOT visit treatment falls within the visit window of any other scheduled visit, these visits can be combined, and assessments will not be repeated.
b. The EOMOP visit will have to be performed within ±1 month of the planned Visit 6/Week 39 of the last participant and will be announced approximately 9 months in advance. For the last participant the EOMOP Visit will be Visit 6/Week 39. For all other participants, if the EOMOP visit falls within the visit window of any other scheduled visit, these visits can be combined, and assessments will not be repeated.
c. Unscheduled visits may be performed at any time during the study. Assessments (other than reporting of AE/SAE and concomitant medication) are to be performed at the discretion of the investigator and are reported in the eCRF. Footnote 0 indicates which assessments are mandatory in case an unscheduled visit is performed.
d. In case an unscheduled visit is performed this assessment must be performed and recorded in the eCRF.
e. Only for participants who prematurely discontinue from study intervention and who disagree to continue to perform visits and assessments until EOMOP, survival information will be collected yearly.
f. A serum pregnancy test must be performed in case of a positive urine pregnancy test.
^{g} Transferred electronically by an external service provider.
h. Assessment not collected in eCRF.
i. In case of premature discontinuation of study intervention before Visit 5/Week 26 it is recommended to perform a postbaseline RHC and within 3 days after last dose of study intervention and before initiation of new PH-specific therapies if possible.
j. If the 6MWT and RHC are assessed on the same day the RHC should be performed after the 6MWT.
k. ABG is only mandatory at the premature EOT in case of premature discontinuation before Visit 5/Week 26 and can be done during the RHC.
l. This assessment can only be done at the premature EOT in case of premature discontinuation of study intervention before Visit 6/Week 39.
m. All AE and SAEs will be reported throughout the study from signing of the ICF onwards until the EOS visit or death or lost to follow-up.
n. Optional assessment.

Participants will be randomized in a 1:1 ratio to receive either selexipag or placebo. Randomization will be stratified by PH-specific therapies at baseline (yes vs no).

The duration of individual participation in the study will be different for each participant (between approximately 15 months and up to approximately 3.5 years), and will depend on the time of each participant's individual date of entering the study and the total recruitment time.

The efficacy assessments include, but are not limited to, RHC at Week 26, assessment of exercise capacity, dyspnea, and WHO FC, measurement of the NT-proBNP levels, recording PROs and CROs.

Safety and tolerability will be evaluated throughout the study from signing of the ICF onwards until the EOS visit or date of last contact. Safety and tolerability assessments include review of concomitant medications and AEs, clinical laboratory tests, 12-lead electrocardiogram (ECG), vital signs, physical examination, pulmonary function tests, DL_{CO}, oxygen saturation and pregnancy testing.

There are two Analysis Timepoints:
Analysis Timepoint 1 (final analysis for the primary endpoint and all supportive efficacy endpoints): When all participants have completed the EOMOP visit, or survival information is available (or if participants have discontinued the study prematurely). At this timepoint, the primary analysis of the PVR endpoint will be conducted. Data for all other endpoints will also be analyzed. After the database lock for the MOP is done, the study team will be unblinded.
Analysis Timepoint 2 (additional safety follow-up): When all participants have completed the safety follow-up phone call or discontinued the study prematurely. At this timepoint, the additional safety data collected during the DB extension period will be reported.

2. Scientific Rationale for Study Design

Rationale for the Use of Placebo/Randomization/Stratification/Blinding

A placebo-controlled study conducted in a randomized and DB fashion will be used to evaluate the efficacy of selexipag and will allow to establish the frequency and magnitude of changes in hemodynamic and clinical endpoints that may occur in the absence of active intervention. The use of a placebo control will also allow proper evaluation of any safety related events or abnormalities and disease progression observed during the study and to differentiate between events potentially related to the use of selexipag vs those related to the underlying disease.

The use of background therapy with PH-specific therapies (excluding prostanoids, prostacyclin analogues and non-prostanoid IP receptor agonists) is allowed. In addition, treatment escalation of PH-specific therapies is allowed following the Week 39 visit (Visit 6). Stable treatment for sarcoidosis is also allowed.

Randomization will be used to minimize bias in the assignment of participants to intervention groups, to increase the likelihood that known and unknown participant attributes (e.g., demographic and baseline characteristics) are evenly balanced across intervention groups, and to enhance the validity of statistical comparisons across intervention groups.

Blinded intervention will be used to reduce potential bias during data collection and evaluation of clinical endpoints. Randomization will be stratified by PH-specific therapies at baseline (yes vs no) to enhance the validity of statistical comparisons across intervention groups and to account for a potentially different treatment effect in participants already receiving PH-specific therapies compared to treatment-naive participants.

Rationale for the Duration of the Study

Participants will receive DB selexipag or placebo until EOT. PVR and other hemodynamic endpoints will be assessed by RHC at Week 26, which is considered sufficient to observe hemodynamic changes. Other clinical endpoints, e.g., exercise capacity and PROs will be assessed up to Week 39, and TTCW and rate of hospitalization related to PH-worsening or death will be assessed up to EOMOP.

In order to be able to estimate the effect of treatment initially assigned at baseline, regardless of adherence to the planned course of treatment (intent-to-treat principle), participants who prematurely discontinue the study intervention will be followed up until EOMOP.

Continued treatment with blinded study intervention for approximately 5 months beyond the EOMOP visit until the study results are available will allow investigators to make an informed decision on post-study therapy based on the primary analysis results and the actual intervention received by the study participants.

Rationale for Right Heart Catherization

Precapillary PH is characterized by an increased resistance to blood flow in the pulmonary vasculature quantified by PVR. PVR is determined by RHC. In addition, a historical RHC is allowed, if the historical RHC was performed in accordance with the guidance provided herein.

Study-Specific Ethical Design Considerations: This study is being conducted to evaluate the efficacy and safety of selexipag in participants with SAPH. The total blood volume to be collected from the population over the study period (see Table 17) is considered to be acceptable based upon WHO recommendations.

3. Justification for Dose

Study intervention will be up-titrated to allow each participant to reach their iMTD, in the range of 200 μg to 1600 μg bid; or in the range of 200 μg to 1600 μg qd for participants with moderate hepatic impairment (Child-Pugh B) or who are concomitantly taking moderate CYP2C8 inhibitor(s). Depending on the iMTD, a single dose of study intervention will consist of 1 to 8 tablets (200 μg to 1600 μg). See, Example 1 for the selexipag formulation for each dose.

4. End-of-Study Definition

EOS Visit: For participants who complete study intervention up to the EOT visit, approximately 5 months after EOMOP, the EOS visit corresponds to the safety follow-up TC (EOS). For participants who prematurely discontinue study intervention between the EOMOP and EOT visits for any reason (except for withdrawal from the study), the EOS visit corresponds to the safety follow-up TC (EOS). For participants who prematurely discontinue study intervention for any reason before the EOMOP visit (except for withdrawal from the study) but complete the main observation period, the EOS visit corresponds to the last visit/TC, which is either the EOMOP visit or the safety follow-up TC, whichever occurs last. For participants who prematurely discontinue study intervention for any reason before the EOMOP visit and who decline to continue with visits and assessments up to the EOMOP visit, but agree to the collection of long-term survival information, the EOS visit corresponds to the last visit or telephone call before study discontinuation. See, Tables 14 and 15.

5. Participant Completion

A participant will be considered to have completed the study if he or she has completed the main observation period (i.e., visits and assessments up to and including the

EOMOP visit), whether or not on study intervention. Participants who prematurely discontinue study intervention and withdraw from the study will be considered to have not completed the study.

Study Completion: The End-of-Study (EOS) is considered as the last visit or EOS visit, whichever occurs last, for the last participant in the study.

C. Study Population

1. Inclusion Criteria

Each potential participant must satisfy all of the following criteria to be enrolled in the study:
Male or female.
18 (or the legal age of consent in the jurisdiction in which the study is taking place) to 75 years of age, inclusive, at Screening.
Confirmed diagnosis of sarcoidosis as per ATS criteria.
Sarcoidosis-associated precapillary PH, confirmed by RHC (at rest) within 90 days prior to randomization:

a. PVR≥320 dyn*sec/cm⁵ (≥4.0 Wood units)

b. Mean pulmonary artery pressure (mPAP)≥25 mmHg

c. Pulmonary artery wedge pressure (PAWP)≤15 mm Hg, or if not available or unreliable, a LVEDP≤15 mmHg. A historical RHC is allowed taking into account the restrictions provided herein. If no historical results are available, an RHC must be performed during the Screening period, but only if all other inclusion criteria are met and none of the exclusion criteria is met.
PH severity according to modified WHO FC II-IV at Screening and randomization; participants of WHO FC IV must be in a stable condition and able to perform a 6MWT.
No treatment with PH-specific therapies or oral PH-specific monotherapy (i.e., riociguat or PDESi or ERA); if on oral PH-specific monotherapy then treatment had to be stable (i.e., no introduction of new therapies or changes in dose) during at least 90 days prior to randomization and the RHC qualifying for enrollment.
Stable sarcoidosis treatment regimen, i.e., no new specific anti-inflammatory treatment for sarcoidosis for at least 90 days, and stable dose(s) for at least 30 days prior to randomization and the RHC qualifying for enrollment.
6MWD between 50 and 450 m both at Screening and at time of randomization.
Forced vital capacity (FVC)>50% of predicted at Screening. Short-acting-beta-agonists (e.g., salbutamol) and long-acting-beta-agonists must not be taken 6 hours and 24 hours prior to spirometry testing, respectively.
FEV₁/FVC≥60%, or if FEV1/FVC<60% then FEV₁ must be ≥60% of predicted at Screening. Short-acting-beta-agonists (e.g., salbutamol) and long-acting-beta-agonists must not be taken 6 hours and 24 hours prior to spirometry testing, respectively.

For patients enrolled or planned to be enrolled in a cardio-pulmonary rehabilitation program based on exercise training, one of the following must apply:

(a) In the maintenance phase of the program at the time of randomization with no plans to stop the program until Week 39, or

(b) Start of the cardio-pulmonary rehabilitation program based on exercise training is planned after Week 39.
A woman must be:

(a) Not of childbearing potential

(b) Of childbearing potential and
Have a negative highly sensitive serum (β-human chorionic gonadotropin [β-hCG]) at Screening and a negative urine pregnancy test at randomization.
Agree to undertake monthly urine pregnancy tests during the study and up to at least 30 days after study intervention discontinuation.
Practicing an acceptable method of contraception and agree to remain on an acceptable method while receiving study intervention and until 30 days after last dose of study intervention.
   A woman using oral contraceptives must have been using this method for at least 1 month prior to randomization.
   Willing and able to adhere to the lifestyle restrictions specified herein.

2. Exclusion Criteria

Any potential participant who meets any of the following criteria will be excluded from participating in the study:
PH due to left heart disease (PAWP>15 mmHg).
PH due to compression of pulmonary arteries and/or pulmonary veins.
History of LHF including cardiomyopathies, cardiac sarcoidosis, with a LVEF<40%.
Severe CHD or unstable angina.
Myocardial infarction within the last 6 months prior to or during Screening.
Decompensated cardiac failure if not under close supervision.
Arrhythmias assessed as severe.
ICD for secondary prevention.
Cerebrovascular events (e.g., transient ischemic attack, stroke) within the last 90 days prior to or during Screening.
Congenital or acquired valvular defects with clinically relevant myocardial function disorders not related to PH.
Overt features of pulmonary veno-occlusive disease (PVOD).
Significant emphysema.
Known and documented severe hepatic impairment (Child-Pugh C).
Severe renal failure (eGFR<30 mL/min/1.73 m² or serum creatinine>2.5 mg/dL) based on central laboratory results from the Screening blood sample.
Treatment with prostacyclin, prostacyclin analogues or IP receptor agonists (i.e., selexipag) during 90 days prior to randomization and/or prior to the RHC qualifying for enrollment, except those given at vasodilator testing during RHC.
Included on a lung transplant list or planned to be included until Visit 6/Week 39.
Known or suspected uncontrolled thyroid disease.
Treatment with moderate inducers of CYP2C8, e.g. rifampicin or strong inhibitors of CYP2C8, e.g., gemfibrozil at or within 14 days prior to randomization.
Change in dose or initiation of new diuretics and/or calcium channel blockers within 1 week prior to RHC qualifying for enrollment.
SBP<90 mmHg at Screening or at randomization.
Known allergies, hypersensitivity, or intolerance to selexipag or its excipients.
Planned or current treatment with another investigational treatment up to 90 days prior to randomization.
Received an investigational intervention or used an invasive investigational medical device within 90 days prior to randomization.
Any condition for which participation would not be in the best interests of the participant (e.g., compromise well-being), or that could prevent, limit, or confound the protocol-specified assessments.
Any acute or chronic impairment that may influence the ability to comply with study requirements such as to perform RHC, a reliable and reproducible 6MWT (e.g., use of walking aids (cane, walker, etc.), or lung function tests.
Employee of the investigator or study site, with direct involvement in the proposed study or other studies under the direction of that investigator or study site, as well as family members of the employees or the investigator.
Pregnant or breast-feeding, or planning to become pregnant while enrolled in this study or within 30 days after the last dose of study intervention

D. Study Intervention

1. Description of Study Interventions

Selexipag 200 μg and matching placebo will be provided.

2. Study Intervention Administration

The tablets are administered orally and should be swallowed whole (i.e., not crushed, split or chewed) with water. Dosing frequency will be bid, except for participants with moderate hepatic impairment (Child Pugh B) or who are concomitantly taking (a) moderate CYP2C8 inhibitor(s), for whom the dosing frequency is qd. In case of bid dosing, at the beginning of each up-titration step, the first dose is to be taken in the evening. In case of qd dosing, participants must take the study intervention in the morning; hence the first dose of study intervention should be taken in the morning of Day 2. Beginning with the morning of Visit 3, and at each visit thereafter up to the EOMOP visit, participants must take the morning study intervention dose before any visit-related procedure and preferably at the site. Tolerability may improve when study intervention is taken with food.

3. Study Intervention Up-titration

Study intervention will be up-titrated to allow each participant to reach their iMTD, in the range of 200 μg to 1600 μg (i.e., 1 to 8 tablets) bid/qd. Dosing frequency will be bid, except for participants with moderate hepatic impairment (Child-Pugh B) or who are concomitantly taking (a) moderate CYP2C8 inhibitor(s), who receive study intervention qd.

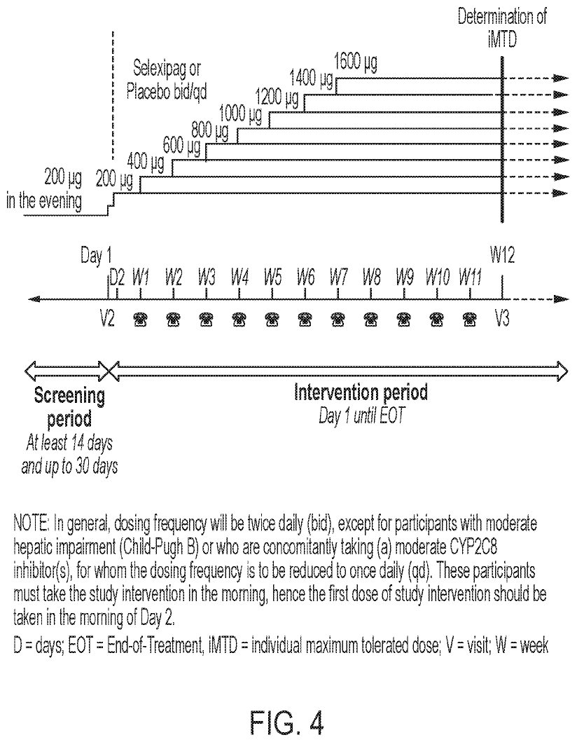

Each participant will start with 1 tablet of DB study intervention, i.e., selexipag (200 μg) or matching placebo, in the evening of Day 1 and will continue with 200 μg bid on Day 2. Participants with moderate hepatic impairment (Child-Pugh B) or who are concomitantly taking (a) moderate CYP2C8 inhibitor(s) will start with 200 μg qd in the morning of Day 2. The dose will be up-titrated in 200 μg bid/qd increments at weekly intervals during scheduled TCs until reaching the iMTD. See, Table 16 and FIG. 4.

TABLE 16
Study Intervention Up-titration Scheme

| Period | Dose regimen | Duration | |
|---|---|---|---|
| First dose | 200 μg | On Day 1 in the evening (pm) | 1 tablet |
| Up-titration | 200 μg bid* | From Day 2 am to Day 8 am** | 1 tablet bid^{§} |
| | 400 μg bid*, ^{§} | *From Day 8 pm to Day 15 am** | 2 tablets bid^{§} |
| | 600 μg bid*, ^{§} | From Day 15 pm to Day 22 am** | 3 tablets bid^{§} |
| | 800 μg bid*, ^{§} | From Day 22 pm to Week 4 am** | 4 tablets bid^{§} |
| | 1000 μg bid*, ^{§} | From Week 4 pm to Week 5 am** | 5 tablets bid^{§} |
| | 1200 μg bid*, ^{§} | From Week 5 pm to Week 6 am** | 6 tablets bid^{§} |
| | 1400 μg bid*, ^{§} | From Week 6 pm to Week 7 am** | 7 tablets bid^{§} |
| | 1600 μg bid*, ^{§} | From Week 7 pm to Week 12 am** | 8 tablets bid^{§} |
| Maintenance | iMTD: | From Week 12 onwards | 1-8 tablets bid^{§} |
| | 200 μg to 1600 μg bid^{§} | | |

am = morning; pm = evening:
*Or the iMTD until Week 12.
**If the dose regimen is not well tolerated or symptoms cannot be fully managed with symptomatic treatment, the duration of the titration step can be prolonged to 2 weeks.
^{§}For participants with moderate hepatic impairment (Child-Pugh B) or who are concomitantly taking (a) moderate CYP2C8 inhibitor(s) the dosing frequency is qd. During the DB period, these participants must take the study intervention in the morning: hence the first dose of study intervention should be taken in the morning of Day 2. During the OL period, these participants can take the first dose of study intervention at a random timepoint during the day, and study intervention will be administered preferably at the same timepoint thereafter.

If the dose regimen is not well tolerated or symptoms cannot be fully managed with symptomatic treatment, the duration of the titration step can be prolonged to 2 weeks. If needed, the dose can be reduced by 200 μg bid/qd. At Week 12 (Visit 3), the bid/qd dose reached for each participant is defined as the iMTD. This dose must be kept stable at least until Week 39 (Visit 6). Any study intervention interruption of 3 days or more will require a new up-titration to avoid tolerability-limiting side effects.

After Week 39, it will be allowed to up-titrate the dose of the participant further if needed (up to the maximum of 1600 μg bid/qd, as applicable) in 200 μg bid/qd increments at scheduled or unscheduled visits. Starting with Visit 3 and at all consecutive

Visits up to and including the EOMOP visit, 6MWT and RHC must be performed within 2 to 5 hours post-dose.

E. Measures to Minimize Bias: Randomization and Blinding

1. Intervention Allocation

Procedures for Randomization and Stratification: Central randomization will be implemented in this study. Participants will be randomly assigned to 1 of 2 intervention groups based on a computer-generated randomization schedule prepared before the study. The randomization will be balanced by using randomly permuted blocks and will be stratified by PH-specific therapy at baseline (yes vs no).

Blinding: The study will be performed in a DB fashion.

2. Study Intervention Compliance

Study intervention compliance is based on study intervention accountability. Study intervention compliance will be calculated by site personnel at each visit using the below formula:

$$\frac{( number of tablets dispensed - number of tablets returned )}{\begin{matrix}total number of tablets that \\ should have been taken during the period\end{matrix}} ⨯ 100$$

3. Concomitant Therapy

Prestudy therapies administered up to 30 days before first dose of study intervention and any previous therapies for the treatment of PH (ERA, PDE5i, riociguat) and sarcoidosis (e.g., infliximab, methotrexate, azathioprine, hydroxychloroquine, leflunomide, steroids) administered up to 90 days before the baseline RHC and 90 days before randomization, respectively, must be recorded at time of Screening.

Concomitant medications, except those listed below, are allowed. Concomitant therapies must be recorded throughout the study from signing of the ICF onwards until the EOS visit or date of last contact. All therapies (oxygen supplementation, prescription or over-the-counter medications, including vaccines, vitamins, herbal supplements; non-pharmacologic therapies such as electrical stimulation, acupuncture, special diets, exercise regimens) different from the study intervention must be recorded. The following medications and/or therapies are not allowed to be administered concomitantly with study intervention:
Prostacyclin (epoprostenol), prostacyclin analogues (e.g., treprostinil, iloprost, beraprost) or prostacyclin receptor agonists (i.e., selexipag/UPTRAVI®) until study intervention discontinuation.
Strong inhibitors of CYP2C8 (e.g., gemfibrozil) until study intervention discontinuation.
Change in dose or initiation of new diuretics and/or calcium channel blockers within 1 week prior to RHC qualifying for enrollment.
Any other investigational drug up to 30 days after study intervention discontinuation.

Medications and/or therapies allowed to be administered concomitantly with study intervention, include, but are not limited to:
Stable doses of therapies for the treatment of PH (i.e., riociguat, PDE5i, ERA). Change in dose or initiation of new therapies for the treatment of PH are allowed as of Week 39 (Visit 6).
Short-acting-beta-agonists (e.g., salbutamol) and long-acting-beta-agonists, but must not be taken within 6 hours and 24 hours prior to spirometry testing, respectively.
Single administration of prostacyclin or analogues used for acute vasodilator testing during an RHC procedure.
Moderate inhibitors of CYP2C8. Dosing frequency of study intervention must be reduced to qd if a moderate inhibitor of CYP2C8 (e.g., clopidogrel, deferasirox, teriflunomide, leflunomide) is concomitantly administered. Dosing frequency of selexipag should be reverted to bid when co-administration of moderate CYP2C8 inhibitor is stopped.
Sarcoidosis-specific treatment, e.g., infliximab, methotrexate, azathioprine, hydroxychloroquine, leflunomide, steroids. Initiation of new treatment or change in dose is allowed as of Week 39 (Visit 6).
Strong inhibitors of UGT1A3 and UGT2B7 (e.g., valproic acid, probenecid, and fluconazole). The effect of strong inhibitors of UGT1A3 and UGT2B7 on the exposure to selexipag and its active metabolite has not been studied. Caution is required when administering these medicinal products concomitantly with selexipag. A potential pharmacokinetic interaction with strong inhibitors of UGT1A3 and UGT2B7 cannot be excluded.

4. Dose Modification

For participants who are unable to tolerate the protocol-specified dosing scheme, dose adjustments must follow the down-titration instructions.

F. Discontinuation of Study Intervention and Participant

Discontinuation/Withdrawal

1. Discontinuation of Study Intervention

A participant's study intervention must be discontinued if:
For safety or tolerability reasons (e.g., AE) it is in the best interests of the participant to discontinue study intervention.
The participant becomes pregnant.
Hepatic impairment occurs or is suspected. If hepatic impairment is suspected, a clinical assessment of severity (Child-Pugh score) should be performed. If a participant has developed severe hepatic impairment (Child-Pugh C) at any time during the study, the study intervention must be permanently discontinued.
Pulmonary edema due to PVOD occurs. Should signs of pulmonary edema occur, the possibility of associated PVOD should be considered. If confirmed, the study intervention must be discontinued.
Study intervention interruptions exceeding 14 consecutive days.
The participant withdraws consent to receive study intervention.

If a participant discontinues study intervention prematurely before the

EOMOP visit, he/she will continue to perform the visits and assessments as scheduled until the EOMOP visit, provided the participant's consent for this limited participation in the study has not been withdrawn. The participant will be asked to return for the premature EOT visit within 7 days of last intake of study intervention and for a safety follow-up phone call 30 (+5) days after the last intake of study intervention. If the premature EOT visit falls within the visit window of any other scheduled visit, these visits can be combined, and assessments will not be repeated.

If a participant discontinues study intervention prematurely before the EOMOP visit and does not agree to continue to perform the visits and assessments as scheduled until the EOMOP visit, he/she will be asked to return for the premature EOT visit within 7 days of last intake of study intervention, for a safety follow-up phone call 30 (+5) days after the last intake of study intervention, provided the participant's consent for this limited participation in the study has not been withdrawn. Long-term survival follow-up information will be collected yearly until death or the time of EOMOP. See, Tables 14 and 15.

Temporary Discontinuation: Study intervention may be temporarily interrupted in response to an AE, a diagnostic or therapeutic procedure, a laboratory abnormality, or for administrative reasons. Interruptions of study intervention must be kept as short as possible. Any study intervention interruption of 3 days or more will require a new up-titration starting with 1 tablet of study intervention (200 μg) bid/qd. For each participant, the up-titration frequency will be up to the medical judgment of the investigator and based on his/her clinical evaluation of the participants' tolerability of the study drug prior to its interruption. Re-uptitration to a previously reached dose can be done at scheduled or unscheduled phone calls or visits, respectively. Study intervention interruptions exceeding 14 consecutive days must lead to permanent discontinuation of study intervention.

2. Participant Discontinuation/Withdrawal From the Study

A participant will not be automatically withdrawn from the study if they have to discontinue study intervention before the end of the intervention regimen. A participant will be withdrawn from the study for any of the following reasons:
Lost to follow-up.
Withdrawal of consent.
Death.
Participant is poorly compliant with study procedures, visits, and assessments.
It is considered that continued participation in the study would be contrary to the best interests of the participant.
Decision for any reason, including, but not limited to premature termination or suspension of the study.

G. Study Assessments and Procedures

1. Overview

Tables 14 and 15 summarize the frequency and timing of efficacy, safety, and biomarker measurements applicable to this study. If it is not possible to complete all assessments on the same day, a visit may extend over more than 1 day within the allowed time window. The following order of assessments is recommended, as applicable:
PROs completed at each site Visit: SF-12, King's SQ, PGA-S

Investigator to complete CGI-S and CGI-C
Safety assessments
Pulmonary function tests
WHO FC
6MWT* and oxygen saturation and BDI (pre- and post-6MWT)
Blood samples for hematology and clinical chemistry tests, NT-proBNP, and/or biomarkers

Arterial Blood gas analysis
RHC*

*Assessments to be performed within 2 to 5 hours post-dose.

The maximum total blood volume to be collected during the entire study from each participant will vary depending on when the patient enters the study. The maximum total blood volume (including the serum beta-hCG pregnancy test and arterial blood gas) to be collected at each visit from screening to Week 39/Visit 6 and from Week 65/Visit 7 until EOT will be approximately 26 mL and 12 mL, respectively (see Table xx). An additional 1 mL to 3 mL of blood may be collected during RHC to measure CO according to Fick's method.

TABLE 17
Volume of Blood to be Collected From Each Participant per Visit

| | | | |
|---|---|---|---|
| | | | Volume per |
| | | Volume per | sample (mL) |
| | Volume | sample (mL) | at each |
| | per | at each | Visit from |
| | Sample | Visit until | Visit 7 to |
| Type of Sample | (mL) | Visit 6^{e} | EOT^{e} |

Safety (including Screening and post-intervention assessments)

| | | | |
|---|---|---|---|
| Hematology | 2 | 2 | 2 |
| Serum chemistry^{b} | 7.5 | 7.5 | 7.5 |
| NT-proBNP | 2.5 | 2.5 | 0 |
| Serum β-hCG | 2.5 | 2.5 | 2.5 |
| pregnancy tests^{d} | | | |
| Exploratory | 4.5 | 4.5 | 0 |
| biomarker plasma | | | |
| sample^{c} | | | |
| Exploratory | 5 | 5 | 0 |
| biomarker serum | | | |
| sample^{c} | | | |
| Arterial Blood | 2 | 2 | 0 |
| Gas^{f} | | | |
| Approximate Total | | 21.5 | 9.5 |
| per Visit^{a} | | | |

^{a}The approximate total per visit does not include the required volume for the serum β-hCG pregnancy test and the arterial blood gas.
^{b}Optional sample.
^{c}Only for women at screening or in case of a positive urine pregnancy test, as no serum β-hCG pregnancy test will be performed in male participants.
^{d}Repeated or unscheduled samples may be taken for safety reasons or technical issues with the samples.
^{e}Arterial blood gas only at screening and Week 26.
Note:
An indwelling intravenous cannula may be used for blood sample collection.

2. Demographics and Baseline Characteristics

Demographic and baseline characteristic data to be collected on all randomized participants include: age, sex, race and ethnicity (where local regulations permit), weight and height, date of the initial SAPH diagnosis, WHO FC, and smoking status (never, former, current).

For participants who are naive to PH-specific therapies, the reason(s) for not prescribing ERA/PDE-5 inhibitors/riociguat are collected.

3. Efficacy Assessments

(a) Primary Efficacy Endpoint Assessment

(i) Right heart catheterization

The following hemodynamic variables are to be collected: HR, peripheral systolic and diastolic blood pressure, pulmonary artery wedge pressure (PAWP; alternatively, LVEDP), mRAP, systolic/diastolic pulmonary artery pressures (sPAP/dPAP), CO, and mixed venous oxygen saturation (SvO₂).

(ii) Baseline RHC: All participants must have a baseline RHC. A historical RHC is allowed, if following criteria are met:

All required variables as per RHC guidance document are available.

It is feasible to perform the Week 26 RHC as described in the RHC guidance document.

Historical RHC was performed:

Within 90 days prior to randomization,

At least 90 days after last change in PH-specific therapies (i.e., change in dose or initiation of new class of drugs).

At least 90 days after starting a new specific anti-inflammatory treatment for sarcoidosis.

At least 30 days after last change in dose of specific anti-inflammatory treatment for sarcoidosis.

If no historical results meeting the above criteria are available, an RHC must be performed during the Screening period only if all other inclusion criteria are met and none of the exclusion criteria is met.

(iii) Week 26 RHC

At Week 26 an RHC will be performed within 2 to 5 hours post-dose. It is recommended that any post-baseline RHC is performed by the same operator, according to the same standards and procedures and in the same catheterization laboratory. Guidance for the RHC assessment is provided in the RHC guidance document.

If a participant prematurely discontinues study intervention between Week 12 and Week 26, it is recommended to perform an RHC within 3 days after last study intervention dose.

(b) Exploratory Endpoints Assessments

(i) Clinical Worsening and Hospitalization Related to PH-worsening: The following data (obtained as part of other assessments) are required to derive TTCW: reporting of death, non-planned PH-related hospitalization, WHO FC, concomitant medications AEs and SAEs. The following cases will be adjudicated by the CEC: hospitalization, increase in WHO FC and initiation of new PH-therapies. For all participants, assessment and reporting of any further disease progression will continue up to the EOMOP visit.

(ii) Survival Follow-up: For participants who prematurely discontinue study intervention and do not agree to continue to perform the visits and assessments up to EOMOP, long-term survival information will be collected yearly until death or the time of EOMOP. Vital status (including date and cause of death) will be collected.

(iii) Exercise Capacity:

6MWT. During the entire study, the 6MWT must be performed within 2 to 5 hours post-dose and prior to the RHC (if applicable). The time of the study intervention morning dose intake will be recorded. For participants who have never performed a 6MWT previously, a training test will be requested before the Screening 6MWTs for inclusion.

Daily Life Physical Activity (DLPA) and sleep parameters: DLPA and sleep parameters are assessed via an actigraphy device. The device is given to the participant at Visit 1 (Screening Visit), and the participant is instructed to wear the actigraphy device on the wrist for 2 weeks during the screening period and following site visits. Detailed instructions for use of the actigraphy device and accessories are provided to participants via a participant guide. The actigraphy device does not display collected data, i.e., the participants do not have access to their activity measurements, as this could influence their behavior. Data will be uploaded to the vendor as described in the participant guide. The data received will be monitored for compliance. Should corrective actions be necessary, participants may be contacted from the site staff. The participants will be provided with a report summarizing their individual activity levels during the study. The data from the actigraphy device will be collected by the vendor, who will send the results to the sponsor.

(iv) Oxygen Saturation (Pre- and Post-6MWT): Measured using pulse oximetry (performed as per standard practice at the study site) just before starting and immediately after stopping the 6MWT.

(v) Dyspnea (Pre- and Post-6MWT): Assessed by the Borg CR10® scale as described in Borg, G 1998. Borg's Perceived Exertion and Pain Scales. Champaign,

Dyspnea will be evaluated by each individual participant immediately before starting and after stopping the 6MWT.

(vi) WHO FC: Assessed as described herein and understood in the art.

(vii) NT-proBNP: A blood sample will be drawn.

(viii) Patient-reported Outcomes:
PAH-SYMPACT™ questionnaire: The PAH-SYMPACT™ questionnaire is a PRO instrument that was developedby Actelion Pharmaceuticals Ltd. The PAH-SYMPACT™ should be completed in the evening before bedtime. This questionnaire has been developed and validated for use in PAH patients. The PAH-SYMPACT™ consists of 2 parts:
   The symptom part is completed for the 7 consecutive days following the visit at site (i.e., starting the day following the visit day).
   The impact part is completed once, on the seventh day of the symptoms diary data collection period, together with the symptom part (i.e., in the evening).
SF-12: The SF-12v2® Healthy Survey is a quality of life self-assessment derived from the SF-36v2® (Health Survey ©1996, 2000 by Medical Outcomes Trust and Quality Metric Incorporated). It has 12 items measuring 8 scales, including physical functioning, physical role limitations, bodily pain, general health, vitality, social functioning, emotional role limitations, and mental health. The SF-12 is scored using norm-based scoring in the same way as for the SF-36v2®.
King's Sarcoidosis questionnaire (KSQ): The KSQ is a health status questionnaire developed and validated for patients with sarcoidosis. It is brief, adaptable to individual patients and assesses general and organ-specific health status. The KSQ consists of 5 modules: General health status (10 items), Lung (6 items), Skin (3 items), Eye (7 items), Medications (3 items). The organ-specific modules can be combined with the General health status module to assess overall health status for patients depending on which organs are affected by their disease. KSQ is summarized as a number between 1 and 100, with higher numbers indicating better health.
Patient Global Assessment of Disease Severity (PGA-S): The PGA-S is a 6-point single item self-evaluation scale. Participants will be asked to rate the overall severity of their disease on the day of administration, with responses of none, very mild, mild, moderate, severe or very severe.
Clinician Global Impression of Severity (CGI-S): The CGI-S is a 6-point single-item scale. The overall severity of the participant's disease will be rated on the day of administration with responses of none, very mild, mild, moderate, severe or very severe.
Clinician Global Impression of Change (CGI-C): The CGI-C is a 7-point single-item scale. The overall participant's change of the participant's disease will be rated since baseline with responses of very much better, moderately better, a little better, no change, a little worse, moderately worse or very much worse.

4. Safety Assessments

Safety and tolerability will be evaluated throughout this study from signing of the ICF onwards until the EOS visit or date of last contact. The standard safety assessments to evaluate the safety and tolerability of selexipag in this study include reporting and follow-up of SAEs, pregnancies, vital signs, physical examination, ECG, and safety laboratory tests. In addition, pulmonary function, DL_{CO} and arterial blood gas will be monitored. The study will include the following evaluations of safety and tolerability according to the timepoints provided in Tables 14 and 15.

(i) Physical Examination: Physical examination at Screening and at subsequent visits includes the examination of the general appearance, heart, lungs, extremities, eyes. Other exams will be performed if indicated, based on medical history and/or symptoms. Height will be measured without shoes. Body weight will be measured in indoor clothing but without shoes.

(ii) Vital Signs: Pulse rate, SBP and DBP will be assessed in a supine or sitting position. It is recommended that the participant is allowed to rest for at least 5 minutes before the measurement. It is also recommended that measurements are performed on the same arm and in the same position (supine or sitting) throughout the study for each individual participant. Vital signs are to be measured prior to blood collection.

(iii) Electrocardiogram: A single standard 12-lead ECG will be performed and interpreted locally. During the collection of ECGs, participants should be in a quiet setting without distractions (e.g., television, cell phones). Participants should rest in a supine position for at least 5 minutes before ECG collection and should refrain from talking or moving arms or legs. If blood sampling or vital sign measurement is scheduled for the same timepoint as ECG recording, the procedures should be performed in the following order: ECG(s), vital signs, blood draw.

(iv) Clinical Safety Laboratory Assessments: Blood samples for serum chemistry and hematology will be collected.

(v) Pulmonary Function Tests
Spirometry: Spirometry tests will be conducted according to the ATS/ERS. It is recommended that all spirometry assessments are performed at the same time and under same conditions throughout the study. Participants must refrain from taking short-acting-beta-agonists (e.g., salbutamol) for 6 hours and long-acting-beta-agonists for 24 hours prior to spirometry testing. If taken, the test should be rescheduled. To perform the spirometry test, participants will be rested for a minimum of 5 minutes prior to start. The following variables will be collected at Screening (Visit 1) and subsequent Visits where applicable: FEV₁, FVC, TLC.
Diffusing Capacity of the Lung for Carbon Monoxide (D_{LCO}): The diffusing capacity of the lungs, measured using carbon monoxide (D_{LCO}) will be conducted according to the ATS/ERS guidelines and will be assessed by the single-breath method. D_{LCO} efforts, up to a maximum of 5, will be performed to produce at least 2 technically acceptable and repeatable traces (according to ATS/ERS guideline criteria). There must be a minimal interval of at least 4 minutes between each effort performed.

(vi) Resting Arterial Blood Gas: Measurement of resting arterial blood gas (ABG) will be performed as per site standard. It is recommended to be performed during RHC if possible. The Week 26 ABG should be performed under the same conditions, e.g., same oxygen rate (if applicable), as the ABG performed at screening. If a historical RHC, is available and if ABG was part of that RHC, it is not necessary to repeat ABG during screening. Values to be recorded include PaCO₂ (mmHg), PaO₂ (mmHg), SaO₂ (mmHg), fraction of inspired oxygen (FiO₂, %) and barometric pressure (BP) on the day of the assessment.

5. Adverse Events and Serious Adverse Events

Adverse events will be reported throughout the study from signing of the ICF onwards until the EOS visit or date of last contact.

(i) Time Period and Frequency for Collecting Adverse Event and Serious Adverse Event Information

All Adverse Events: All AEs and special reporting situations, whether serious or non-serious, will be reported throughout the study from signing of the ICF onwards until the EOS visit or date of last contact.

Serious Adverse Events: All SAEs occurring during the study must be reported within 24 hours of their knowledge of the event. Serious adverse events, including those spontaneously reported throughout the study from signing of the ICF onwards until the EOS visit or date of last contact, must be reported, irrespective of the study intervention received by the participant and whether or not this event is considered to be related to study intervention. Serious adverse events occurring after the EOS visit must be reported only if considered to be causally related to previous exposure to the study intervention.

(ii) Pregnancy: All initial reports of pregnancy in female participants must be reported within 24 hours of their knowledge of the event. Pregnancies must be reported as an AE and any AE associated with the pregnancy occurring until the EOS visit or date of last contact must be reported as a separate AE.

(iii) Treatment of Overdose: Overdose is defined by the intake of any single dose greater than 1600 μg or a total daily dose greater than 3200 μg (only in case subjects are on a bid regimen).

(iv) Biomarkers: Biomarker analysis is optional for each participant.

Plasma and serum samples for the analysis of pharmacodynamic and disease-related biomarkers can be collected at timepoints indicated in Tables 14 and 15.

H. Statistical Considerations

Data collected during the study will be summarized by intervention group and, where appropriate and applicable, compared between intervention groups with hypothesis test.

1. Populations for Analyses:

For analysis purposes, the populations are defined in Table 18:

TABLE 18
Overview of the different populations for analyses

| Population | Description |
|---|---|
| Screened Analysis Set (SCR) | All participants who were screened and received a participant |
| | identification number. |
| Randomized Set (RS) | All participants assigned to a study intervention. |
| Full Analysis Set (FAS) | All patients from the RS who took at least one dose of study |
| | intervention and for whom the baseline pulmonary vascular |
| | resistance is non-missing. |
| Per-Protocol Analysis Set | All participants from the FAS who received the assigned |
| (PPS) | study intervention and who complied with the protocol |
| | sufficiently to be likely to exhibit the intervention effects. |
| Safety Analysis Set (SAS) | All participants who received at least one dose of study |
| | intervention. Participants will be analyzed according to the |
| | intervention they actually received. |

PVR and all other efficacy analyses will primarily be evaluated on the FAS. The PVR analyses will also be performed on the PPS, unless the number of subjects in the PPS and the FAS differ by less than 5%, in which case, no analyses will be performed using the PPS. Safety endpoints will be primarily evaluated on the SAS.

2. Analyses Timepoints

There are 2 Analysis Timepoints.

3. Sample Size Determination

The calculation of sample sizes was approximated by Z-test for superiority on the log-transformed PVR post- to pre-percent of baseline using the software package EASTTM version 6.4.0.1 for the difference of two means. In order to guarantee the appropriate design characteristics, a total of 74 patients is required (i.e., 37 patients per treatment arm). A sample size of 74 subjects (37 per treatment arm) was determined to provide 90% power (with a Type I error of 5%) to detect a clinically relevant 30% relative improvement in the geometric means of the ratio of post-baseline PVR over baseline PVR values between the selexipag and placebo groups (assuming a coefficient of variation of the ratio of 0.5).

Table 19 displays power calculations for different assumptions for these two parameters and a two-sided α=0.05.

TABLE 19
Sample size sensitivity for the main analysis

| Scenario | Ratio of GMs | CV | Power |
|---|---|---|---|
| Sensitivity 1 | 0.65 | 0.45 | 99% |
| Sensitivity 2 | 0.65 | 0.50 | 97% |
| Sensitivity 3 | 0.70 | 0.45 | 94% |
| Original | 0.70 | 0.50 | 90% |
| Sensitivity 4 | 0.75 | 0.45 | 82% |
| Sensitivity 5 | 0.75 | 0.50 | 74% |

CV = coefficient of variation; GM = geometric mean.

Values in bold, italics in Table 19 correspond to the sample size calculation in the paragraph above.

4. Statistical Analyses

(i) Primary Efficacy Analysis

Definition of PVR endpoint: The primary endpoint is PVR at peak concentration up to Week 26, i.e., at Week 26 or at premature EOT in case of discontinuation of study treatment before Week 26.

Estimand

The primary PVR estimand is described according to the following four attributes:

a. Population: FAS (Full analysis set), as randomized.

b. Variable: Per each patient, the absolute change from baseline to Week 26 (or premature EOT) visit of the natural log-transformed PVR values will be estimated. The corresponding ratio of the PVR value post-intervention initiation at Week 26 (or premature EOT) (post) vs the PVR value pre-intervention initiation at baseline (pre), expressed as a percentage will also be estimated, i.e.,:

$$PVR post to pre percent = ( \frac{PVR at Week 26 ( or EOT )}{PVR at baseline} ) ⨯ 100 ( \% )$$

c. Intercurrent events (events that preclude observation of the variable or affect its interpretation):
Death occurring prior to Week 26 (or premature EOT) visit,
Initiation of prostacyclin (epoprostenol), prostacyclin-analogs (e.g., treprostinil, iloprost, beraprost) or prostacyclin receptor agonists (i.e., selexipag/UPTRAVI) for any reason prior to post-baseline RHC,
Study treatment discontinuation/interruption for more than 3 days immediately prior to the post-baseline RHC

d. Population-level summary: Ratio of the geometric mean (GM) of the PVR post to pre percent between selexipag and placebo, obtained by exponentiation of the difference in means of the natural logarithm transformed PVR post to pre percent between selexipag and placebo:

$$\frac{GM ( PVR post to pre percent in selexipag )}{GM ( PVR post to pre percent in placebo )}$$

This estimand targets the effect of treatment initiation on the variable measurement prior to the occurrence of death or introduction of prohibited PH-specific medication and follows a “while on treatment” strategy. For the purpose of the primary endpoint, on-treatment is defined as up to 3 days after last study intervention intake.

Intercurrent events will be handled as follows:
“Death occurring prior to the post-baseline RHC” will be addressed by imputing the post-baseline PVR value using the missing data rules specified in subsequent sections.
Initiation of prostacyclin (epoprostenol), prostacyclin analogs (e.g., treprostinil, iloprost, beraprost) or prostacyclin receptor agonists (i.e., selexipag/UPTRAVI) for any reason prior to post-baseline RHC″ will be addressed by disregarding the post-baseline PVR assessments and imputing them using the missing data rules specified in subsequent sections.
“Study treatment discontinuation/interruption for more than 3 days immediately prior to the post-baseline RHC” will be addressed by disregarding the PVR assessments obtained more than 3 days after study treatment discontinuation and imputing them using the missing data rules specified in subsequent sections.

Hypotheses

The hypotheses for the primary endpoint of PVR at peak concentration at Week 26 (or premature EOT) are formulated in terms of GM of PVR post to pre percent (as defined above) in participants treated with selexipag (GM_{selexipag}) versus placebo (GM_{placebo}):

H0: GM_{selexipag}/GM_{placebo}=1

H_{A}: GM_{selexipag}/GM_{placebo}≠1

Statistical Model

The null hypothesis is planned to be tested in the FAS by means of an ANCOVA model on the natural logarithm transformed PVR post to pre percent. Model covariates will include randomized intervention, stratification factor and the natural logarithm transformed baseline PVR value. The results will be presented on the original scale (GM) after exponentiation of the absolute adjusted mean changes obtained on the natural log scale:

Handling of Missing Data

By design, only one post-baseline PVR measurement will be taken at the time of the scheduled Week 26 or at the premature EOT visit. In some instances, the Week 26 (or premature EOT) assessment may be missing with no other post-baseline assessment available.

Imputation methods for PVR will be specific to the reason for missing data.

The baseline reference value for PVR is based on the last RHC performed prior to study intervention initiation.

If PVR cannot be calculated due to missing PAWP, the following conventions will be applied for the calculation at a visit at which both mPAP and CO are assessed and not missing:

If only PAWP is missing, then the LVEDP will be used.

If PAWP and LVEDP are missing both at baseline and post-baseline, the missing PAWP is imputed using the median of all values observed at the respective time point in all subjects from the same randomized intervention group.

If PAWP and LVEDP are missing either at baseline or at post-baseline, the available PAWP for the subject is used as a substitute for the missing PAWP.

If a post-baseline PVR assessment is missing up to Week 26, the following missing data imputation rules will be used:

If a participant dies without a post-baseline value, then the missing PVR post to pre fold value is imputed with the largest PVR post to pre percent value for post-baseline amongst all participants in the same analysis set. The resulting imputed post-baseline PVR is the product of this imputed PVR post to pre percent value and the respective baseline PVR value.

If a participant experiences clinical worsening, with a subsequent missing PVR post to pre fold value, the post-baseline value is imputed with the 75^{th} percentile of the PVR post to pre fold values from all participants in the same analysis set. The resulting imputed post-baseline PVR is the product of this imputed PVR post to pre fold value and the respective baseline PVR value.

If the participant is alive and does not experience clinical worsening, then the missing PVR post to pre fold value is imputed with the 50^{th} percentile of the PVR post to pre fold values from all participants in the same intervention group and analysis set. The resulting imputed post-baseline PVR is the product of this imputed PVR post to pre fold value and the respective baseline PVR value.

Main Analysis

The analyses of the primary efficacy endpoint will be tested in the FAS using the ANCOVA model.

From the ANCOVA model, PVR post to pre percent will be summarized by intervention group using covariate-adjusted GM and corresponding 2-sided 95% CI. The between-group ratio of GM with corresponding 95% 2-sided CI and p-value will be displayed as the placebo-corrected intervention effect.

For each intervention group, the least square mean and 2-sided 95% CI of the natural logarithm of the PVR post to pre percent will be inversely transformed using the exponential function and multiplied by 100 to provide the GM of the PVR post to pre percent and the corresponding 2-sided 95% CI, expressed as a percentage.

Absolute values at baseline and post-baseline as well as absolute pre-post changes from baseline to post-baseline for PVR will be summarized using descriptive statistics.

Supportive/Sensitivity Analyses

Sensitivity analyses will be conducted using alternative imputation rules to assess the imputation assumptions of the main statistical analysis for PVR at Week 26 (or premature EOT).

Furthermore, a sensitivity analysis of PVR at Week 26 (or premature EOT) will be conducted in the per-protocol analysis set to assess the robustness of the results of the main statistical analysis against protocol deviations leading to exclusion from the PPS.

(ii) Other Efficacy Analyses

For continuous and categorical endpoints, where applicable, the response profiles over time (e.g., change from baseline, proportions of participants by categorical endpoint categories) will be inspected graphically and by the usual summary statistics at each visit (with 95% CIs) up to the EOMOP visit by randomized treatment group on the RS. Two sets of profiles will be provided: one considering assessments measured on study intervention up to the EOMOP visit, and one including all data collected up to the EOMOP visit regardless of treatment discontinuations.

Time-to-event endpoints will be analyzed using the Kaplan-Meier method by randomized treatment group up to EOMOP, regardless of study treatment discontinuations.

(iii) Safety Analyses

Adverse Events: The verbatim terms used to identify AEs will be coded using MedDRA. Intervention-emergent adverse events are AEs with onset during the intervention period or that are a consequence of a pre-existing condition that has worsened since baseline. All reported AEs will be included in the analysis. For each AE, the percentage of participants who experience at least 1 occurrence of the given event will be summarized by intervention group. In addition, comparisons between intervention groups will be provided if appropriate. Summaries, listings, datasets, or participant narratives may be provided, as appropriate, for those participants who die, who discontinue intervention due to an AE, or who experience a severe or an SAE. Summaries will be by intervention (selexipag or placebo) and will include all events collected throughout the study.

Clinical Laboratory Tests: Laboratory data will be summarized by type of laboratory test. Reference ranges and markedly abnormal results will be used in the summary of laboratory data. Descriptive statistics will be calculated for each laboratory analyte at baseline and for observed values and changes from baseline at each scheduled timepoint by intervention group. Frequency tabulations of the abnormalities will be made.

Vital Signs: Descriptive statistics of pulse rate, SBP and DBP values and changes from baseline will be summarized at each scheduled timepoint by intervention group.

Supplemental Oxygen Rate: Descriptive statistics of values and changes from baseline in supplemental oxygen rate will be summarized by intervention group.

Pulmonary Function Tests: Descriptive statistics of values and changes from baseline in DL_{CO} and FVC will be summarized by intervention group.

Arterial Blood Gas: Descriptive statistics of values and changes from baseline in ABG parameters will be summarized by intervention group

(iv) Biomarker Analyses: Biomarker samples will be used to generate plasma and serum marker data for computational analyses. Changes in plasma and serum biomarkers over time will be summarized by intervention group. Associations between baseline levels and changes from baseline in selected markers and clinical responses will be explored.

The examples and embodiments described herein are for illustrative purposes only and various modifications or changes suggested to persons skilled in the art are to be included within the spirit and purview of this application and scope of the appended claims.

1-29. (canceled)

30. A method for treating sarcoidosis-associated pulmonary hypertension, comprising administering to a patient in need thereof a therapeutically effective amount of selexipag.

31. A method for treating sarcoidosis-associated pulmonary hypertension in a patient in need thereof, comprising (a) determining if the patient has sarcoidosis-associated pulmonary hypertension; and (b) if the patient has sarcoidosis-associated pulmonary hypertension, administering a therapeutically effective amount of selexipag.

32. The method of claim 30, wherein within about 90 days of initiating the administration of the selexipag, the patient has a pulmonary vascular resistance (PVR)≥about 240 dyn·s·cm⁻⁵ (≥3 Wood Units), a mean pulmonary arterial pressure (mPAP)≥about 25 mmHg, and a pulmonary arterial wedge pressure (PAWP)≤about 15 mmHg or a left ventricular end diastolic pressure (LVEDP)≤about 15 mmHg.

33. The method of claim 30, wherein, prior to initiating the administration of the selexipag, the patient is in World Health Organization functional class II, III, or IV.

34. The method of claim 33, wherein the patient is in stable condition and able to perform a 6-minute walk test (6MWT) if the patient is in World Health Organization functional class IV.

35. The method of claim 30, wherein the patient is not receiving treatment for pulmonary hypertension, or is receiving stable treatment for pulmonary hypertension for at least about 90 days, prior to initiating the administration of the selexipag.

36. The method of claim 35, wherein the stable treatment for pulmonary hypertension comprises riociguat, a phosphodiesterase type-5 inhibitor, or an endothelin receptor antagonist.

37. The method of claim 30, wherein the patient is not receiving treatment for sarcoidosis for at least about 90 days, or is receiving stable treatment for sarcoidosis for at least about 30 days, prior to initiating the administration of the selexipag.

38. The method of claim 37, wherein the treatment for sarcoidosis comprises anti-inflammatory treatment.

39. The method of claim 30, wherein the patient has a forced vital capacity (FVC)>50%.

40. The method of claim 30, wherein the patient has a 6-minute walk distance (6MWD) between about 50 m and about 450 m prior to initiating the administration of the selexipag.

41. The method of claim 30, wherein, prior to initiating the administration of the selexipag, the patient does not have left heart failure.

42. The method of claim 30, wherein the patient is not being treated with a prostacyclin, a prostacyclin analogue, or a prostacyclin receptor agonist about 90 days prior to initiating the administration of the selexipag.

43. The method of claim 30, wherein the patient does not have Child-Pugh class C liver disease prior to initiating the administration of the selexipag.

44. The method of claim 30, wherein the patient does not have a systolic blood pressure of <about 90 mmHg prior to initiating the administration of the selexipag.

45. The method of claim 30, wherein the selexipag is administered at a starting dose and is increased to determine an individual maximum tolerated dose (iMTD).

46. The method of claim 45, wherein the starting dose is about 200 μg twice daily.

47. The method of claim 45, wherein the iMTD is from about 200 μg to about 1600 μg twice daily.

48. The method of claim 45, wherein the starting dose is increased in twice-daily increments of about 200 μg until the iMTD is determined in a dose adjustment phase.

49. The method of claim 48, wherein the dose adjustment phase is from about 8 to about 12 weeks.

50. The method of claim 45, wherein the iMTD does not exceed about 1600 μg twice daily.

51. The method of claim 48, wherein the iMTD is maintained during a maintenance phase following the dose adjustment phase.

52. The method of claim 51, wherein the maintenance phase is at least about 14 weeks.

53. The method of claim 52, wherein the maintenance phase is at least about 26 weeks.

54. The method of claim 52, wherein a further dose increase takes place following the maintenance phase, but does not exceed about 1600 μg twice daily.

55. The method of claim 30, wherein the method reduces the patient's PVR relative to a patient population at the same or similar level of disease diagnosis that is not receiving treatment with selexipag.

56. The method of claim 30, wherein the method increases the time until first incidence of one or more events selected from all cause death, non-planned pulmonary hypertension hospitalization, increase in WHO functional class, deterioration by at least 15% from baseline in exercise capacity as measured by the 6MWD, lung transplantation, atrial balloon septostomy, initiation of parenteral or new class of PH-specific therapy for clinical worsening, or symptoms of right heart failure, relative to a patient population at the same or similar level of disease diagnosis that is not receiving treatment with selexipag.

57. The method of claim 30, wherein the selexipag is administered orally in the form of a tablet.

58. The method of claim 30, wherein the patient has a FEV₁/FVC of greater or equal to 60%, or if the FEV₁/FVC is less than 60%, then the FEV₁ is greater than or equal to 60%, prior to the administration of the selexipag.
